(19) United States
(12) Patent Application Publication
(10) Pub. No.: US 2026/0250371 A1
(43) Pub. Date: Aug. 27, 2026

(54) HUMAN FGF23-BINDING ANTIBODIES WITH IMPROVED AFFINITY AND EFFICACY

(71) Applicant: Ultragenyx Pharmaceutical Inc., Novato, CA (US)

(72) Inventors: Mark Fox, Novato, CA (US); Emil Kakkis, Novato, CA (US); Mika Aoyagi-Scharber, Novato, CA (US); Florian-Alexander Herbst, Hamburg (DE); Katrin Schaefer, Hamburg (DE); Brianna Klein, Princeton, NJ (US); Mark Brooks, Princeton, NJ (US); Christopher Lane Sander, Princeton, NJ (US); Igor D'Angelo, Seattle, WA (US); Lucky Ahmed, Seattle, WA (US)

(21) Appl. No.: 19/163,370

(22) PCT Filed: Mar. 8, 2024
(86) PCT No.: PCT/US2024/019027

§ 371 Date: Sep. 8, 2025

Related U.S. Application Data
(60) Provisional application No. 63/489,458, filed on Mar. 10, 2023.
(60) Provisional application No. 63/514,180, filed on Jul. 18, 2023.
(60) Provisional application No. 63/548,711, filed on Feb. 1, 2024.

(51) Int. Cl. C07K 16/22

(52) U.S. Cl. CPC C07K 16/22; C07K 2317/92

(57) ABSTRACT

The disclosure provides improved human FGF23-binding antibodies with increased affinity for human FGF23 as compared to burosumab, enabling reduced dosing frequency, improving convenience of therapy, and achieving higher efficacy compared to burosumab, without compromising safety.

### CROSS-REFERENCE TO RELATED APPLICATIONS

This application claims the benefit of and priority to U.S. Provisional Application No. 63/548,711, filed 1 Feb. 2024, U.S. Provisional Application No. 63/514,180, filed 18 Jul. 2023, and U.S. Provisional Application No. 63/489,458, filed 10 Mar. 2023, the entire disclosures of which are each hereby incorporated by reference herein in their entireties for all purposes.

### REFERENCE TO A SEQUENCE LISTING XML

This application contains a Sequence Listing which has been submitted electronically in XML format. The Sequence Listing XML is incorporated herein by reference. Said XML file, created Feb. 28, 2024, is named UXB023-06WO_SL.xml, and is 2,280,142 bytes in size.

### TECHNICAL FIELD OF THE DISCLOSURE

This disclosure relates to human FGF23-binding antibodies with improved affinity and improved efficacy compared to burosumab.

### BACKGROUND

Burosumab (i.e., burosumab-twza, marketed as CRYSVITAR) is a fibroblast growth factor 23 (FGF23)-blocking antibody indicated for the treatment of X-linked hypophosphatemia (XLH) in adult and pediatric patients 6 months of age and older and for the treatment of adults and children 2 years of age and older with FGF23-related hypophosphatemia in tumor-induced osteomalacia (TIO) when the tumor cannot be located or removed. Burosumab is a recombinant human IgG1κ monoclonal antibody. Burosumab consists of 4 polypeptide chains (2 identical heavy chains, 447 amino acids each, and 2 identical light chains, 213 amino acids each) and has a molecular weight of 147 kDa. Burosumab binds specifically to FGF23 and blocks its interaction with the Klotho-FGF receptor complex.

FGF23 is a naturally occurring cytokine involved in phosphate and vitamin D metabolism. Excess levels of circulating FGF23 can lead to increased urinary phosphate excretion, reduced vitamin D synthesis, and subsequent hypophosphatemia resulting in defective bone mineralization and impacts to other tissues including muscle. Burosumab binds to FGF23 and inhibits the ability of FGF23 to bind to fibroblast growth factor receptor 1 (FGFR1) and the obligate co-receptor Klotho. This inhibition restores tubular reabsorption of phosphate from the kidney and increases production of vitamin D, which enhances intestinal absorption of calcium and phosphate. These combined actions improve serum phosphorus levels and bone mineralization.

Burosumab administration to pediatric patients is carried out by subcutaneous injection every 2 weeks (Q2W), representing a significant treatment burden. Additionally, both adult and pediatric patients must undergo high dosing volumes of burosumab injection, with a maximum of up to 3 ml administered in three separate 1 ml injections (see, e.g., burosumab-twza prescribing information, available at www.accessdata.fda.gov/scripts/cder/daf/index.cfm). Accordingly, there is a need for human FGF23-binding antibodies with improved affinity and efficacy compared to burosumab.

### BRIEF SUMMARY

The disclosure provides improved human FGF23-binding antibodies with increased affinity for human FGF23 as compared to burosumab, enabling reduced dosing frequency, improving convenience of therapy, and achieving higher efficacy compared to burosumab, without compromising safety.

The present disclosure provides FGF23-binding antibodies engineered to have improved affinity compared to burosumab by changing one or more amino acids in the heavy chain (SEQ ID NO: 1) and/or light chain (SEQ ID NO: 2) sequence(s) of burosumab. In some embodiments, the FGF23-binding antibodies are engineered to have improved affinity compared to burosumab by changing one or more amino acids in the heavy chain variable region (VH, SEQ ID NO: 3) and/or light chain variable region (VL, SEQ ID NO: 4) sequence(s) of burosumab.

In some embodiments, the disclosure provides an isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 but comprising one or more amino acid change relative to SEQ ID NO: 3, wherein the one or more amino acid change is selected from T30E, T30F, T30Q, T30S, T30Y, N31D, N31E, N31F, N31Q, N31R, N31Y, H32E, H32F, H32Q, H32S, H32T, I50D, 150E, I50F, I50S, I50T, 150Y, N52E, N52F, N52Q, N52Y, I54E, I54F, I54L, I54Q, I54S, I54T, I54V, I54Y, S55E, S55F, S55Q, S55T, S55Y, S59D, S59E, S59F, S59I, S59L, S59Q, S59T, S59V, S59Y, D99E, D99Q, D99Y, I100D, I100E, I100F, I100L, I100Q, I100V, I100Y, V101F, V101I, V101L, V101T, V101Y, D102E, D102F, D102I, D102L. D102Q, D102Y, A103D, A103E, A103F, A103Q, A103S, A103T, and A103Y.

In some embodiments, the disclosure provides an isolated antibody or a fragment thereof that specifically binds to a human fibroblast growth factor 23 (FGF23) polypeptide, wherein the antibody or fragment thereof comprises a light chain variable region (VL) amino acid sequence according to SEQ ID NO: 4 but comprising one or more amino acid change relative to SEQ ID NO: 4, wherein the one or more amino acid change is selected from A25L. A25V, I29F, 129L, 129V, S30D, S30E, S30F, S30I, S30L, S30Q, S30T, S30V, S30Y, S31D, S31E, S31F, S31I, S31L, S31Q, S31T, S31V, S31Y, A32F, A32I, A32L, A32T, A32V, A32Y, V34F, V34I, V34L, V34Y, D50E, D50F, D50I, D50L, D50Q, D50R, D50S, D50T, D50V, A51F, A51I, A51L, A51S, A51T, A51V, S52D, S52E, S52F, S52Q, S52T, S52Y, S53D, S53E, S53F, S53I, S53L, S53Q, S53R, S53T, S53V, S53Y, Q90F, Q90Y, F91K, F91R, F91Y, N92D, N92E, N92F, N92Q, N92S, N92T, N92Y, D93E, D93F, D93Q, D93R, D93S, D93T, D93Y, Y94D, Y94E, Y94F, Y94Q, Y94S, and Y94T.

In some embodiments, the disclosure provides an isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VH amino acid sequence comprises one or more amino acid change selected from T30E, T30F, T30Q, T30S, T30Y, N31D, N31E, N31F, N31Q, N31R, N31Y, H32E, H32F, H32Q, H32S, H32T, I50D, 150E, 150F, I50S, 150T, 150Y, N52E, N52F, N52Q, N52Y, I54E, I54F, I54L, I54Q, I54S, I54T, I54V, I54Y, S55E, S55F, S55Q, S55T, S55Y, S59D, S59E, S59F, S59I, S59L, S59Q, S59T, S59V, S59Y, D99E, D99Q, D99Y, I100D, I100E, I100F, I100L, I100Q, I100V, I100Y, V101F, V101I, V101L, V101T, V101Y, D102E, D102F, D102I, D102L, D102Q, D102Y, A103D, A103E, A103F, A103Q, A103S, A103T, and A103Y, and/or wherein the VL amino acid sequence comprises one or more amino acid change selected from A25L, A25V, I29F, I29L, I29V, S30D, S30E, S30F, S30I, S30L, S30Q, S30T, S30V, S30Y, S31D, S31E, S31F, S31I, S31L, S31Q, S31T, S31V, S31Y, A32F, A32I, A32L, A32T, A32V, A32Y, V34F, V34I, V34L, V34Y, D50E, D50F, D50I, D50L, D50Q, D50R, D50S, D50T, D50V. A51F, A51I, A51L, A51S, A51T, A51V, S52D, S52E, S52F, S52Q, S52T, S52Y, S53D, S53E, S53F, S53I, S53L, S53Q, S53R, S53T, S53V, S53Y, Q90F, Q90Y, F91K, F91R, F91Y, N92D, N92E, N92F, N92Q, N92S, N92T, N92Y, D93E, D93F, D93Q, D93R, D93S, D93T, D93Y, Y94D, Y94E, Y94F, Y94Q, Y94S, and Y94T.

In some embodiments, the disclosure provides an isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 and a light chain variable region (VL) according to SEQ ID NO: 4, but comprising at least two amino acid changes in the VH and VL sequences, wherein the at least two amino acid changes are selected from VL:S31E/VL:D50F, VH:N31F/VH:S59F, VL:D50F/VL:S53E, VL:S31F/VL:S52E, VL:D50F/VH:V101I. VH:S59D/VH:V101I, VL:S31F/VL:S53E, VL:N92E/VL:D93F, VL:S31E/VL:S53E, VL:S31F/VL:D50F, VH:N31E/VH:S59E, VH:S57E/VH:S59D, VH:N31E/VH:I54F, VH:S59T/VH:V101L, VH:I54Y/VH:V101F, VH:N52F/VH:V101I, VL:N92D/VL:D93F, VH:N52F/VH:V101L, VL:S31F/VL:D50E, VL:Y94D/VH:S59D, VL:Y94D/VH:S59E, VL:N92F/VH:S59E, VL:N92Y/VH:S59E, VL:Y94E/VH:S59E, VL:S52D/VH:S59E, and VL:S52E/VH: S59E.

Also provided herein are FGF23-binding antibodies having one or more unique complementarity-determining regions (CDRs) relative to burosumab.

In some embodiments, the disclosure provides an isolated antibody or a fragment thereof that specifically binds to a human fibroblast growth factor 23 (FGF23) polypeptide, wherein the antibody or fragment thereof comprises a heavy chain complementarity determining region 1 (HCDR1) according to an amino acid sequence selected from SEQ ID NO: 11 and SEQ ID NOs: 22-32.

In some embodiments, the disclosure provides an isolated antibody or a fragment thereof that specifically binds to a human fibroblast growth factor 23 (FGF23) polypeptide, wherein the antibody or fragment thereof comprises a heavy chain complementarity determining region 2 (HCDR2) according to an amino acid sequence selected from SEQ ID NO: 12 and SEQ ID NOs: 33-65.

In some embodiments, the disclosure provides an isolated antibody or a fragment thereof that specifically binds to a human fibroblast growth factor 23 (FGF23) polypeptide, wherein the antibody or fragment thereof comprises a heavy chain complementarity determining region 3 (HCDR3) according to an amino acid sequence selected from SEQ ID NO: 13 and SEQ ID NOs: 66-93.

In some embodiments, the disclosure provides an isolated antibody or a fragment thereof that specifically binds to a human fibroblast growth factor 23 (FGF23) polypeptide, wherein the antibody or fragment thereof comprises a light chain complementarity determining region 1 (LCDR1) according to an amino acid sequence selected from SEQ ID NO: 14 and SEQ ID NOs: 94-126.

In some embodiments, the disclosure provides an isolated antibody or a fragment thereof that specifically binds to a human fibroblast growth factor 23 (FGF23) polypeptide, wherein the antibody or fragment thereof comprises a light chain complementarity determining region 2 (LCDR2) according to an amino acid sequence selected from SEQ ID NO: 15 and SEQ ID NOs: 127-158.

In some embodiments, the disclosure provides an isolated antibody or a fragment thereof that specifically binds to a human fibroblast growth factor 23 (FGF23) polypeptide, wherein the antibody or fragment thereof comprises a light chain complementarity determining region 3 (LCDR3) according to an amino acid sequence selected from SEQ ID NO: 16 and SEQ ID NOs: 159-185.

In some embodiments, the disclosure provides an isolated antibody or a fragment thereof that specifically binds to a human fibroblast growth factor 23 (FGF23) polypeptide, wherein the antibody or fragment thereof comprises a HCDR1, a HCDR2, a HCDR3, a LCDR1, a LCDR2, and a LCDR3, wherein: the HCDR1 comprises an amino acid sequence selected from SEQ ID NO: 11 and SEQ ID NOs: 22-32; the HCDR2 comprises an amino acid sequence selected from SEQ ID NO: 12 and SEQ ID NOs: 33-65; the HCDR3 comprises an amino acid sequence selected from SEQ ID NO: 13 and SEQ ID NOs: 66-93: the LCDR1 comprises an amino acid sequence selected from SEQ ID NO: 14 and SEQ ID NOs: 94-126; the LCDR2 comprises an amino acid sequence selected from SEQ ID NO: 15 and SEQ ID NOs: 127-158; and the LCDR3 comprises an amino acid sequence selected from SEQ ID NO: 16 and SEQ ID NOs: 159-185.

Also provided herein are FGF23-binding antibodies having one or more unique variable region domains relative to burosumab.

For instance, the isolated antibody or fragment thereof may comprise a heavy chain variable region (VH) and a light chain variable region (VL), wherein the VH comprises an amino acid sequence having at least 80% sequence identity to an amino acid sequence selected from SEQ ID NO: 3 and SEQ ID NOs: 186-270, or wherein the VL comprises an amino acid sequence having at least 80% sequence identity to an amino acid sequence selected from SEQ ID NO: 4 and SEQ ID NOs: 271-368.

The isolated antibody or fragment thereof may comprise a VH comprising an amino acid sequence having at least 90% sequence identity to an amino acid sequence selected from SEQ ID NO: 3 and SEQ ID NOs: 186-270, or the antibody or fragment thereof may comprise a VL comprising an amino acid sequence having at least 90% sequence identity to an amino acid sequence selected from SEQ ID NO: 4 and SEQ ID NOs: 271-368.

The isolated antibody or fragment thereof may comprise a VH comprising an amino acid sequence having at least 95% sequence identity to an amino acid sequence selected from SEQ ID NO: 3 and SEQ ID NOs: 186-270, or the antibody or fragment thereof may comprise a VL comprising an amino acid sequence having at least 95% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence having at least 80% sequence identity to an amino acid sequence selected from SEQ ID NO: 3 SEQ ID NOs: 186-270, and the VL may comprise an amino acid sequence having at least 80% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence having at least 90% sequence identity to an amino acid sequence selected from SEQ ID NO: 3 and SEQ ID NOs: 186-270, and the VL may comprise an amino acid sequence having at least 90% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence having at least 95% sequence identity to an amino acid sequence selected from SEQ ID NO: 3 and SEQ ID NOs: 186-270, and the VL may comprise an amino acid sequence having at least 95% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence selected from SEQ ID NO: 3 and SEQ ID NOs: 186-270, or the VL may comprise an amino acid sequence selected from SEQ ID NO: 4 and SEQ ID NOs: 271-368.

The isolated antibody or fragment thereof may comprise a VH and a VL, wherein the VH may comprise an amino acid sequence selected from SEQ ID NO: 3 and SEQ ID NOs: 186-270, and the VL may comprise an amino acid sequence selected from SEQ ID NO: 4 and SEQ ID NOs: 271-368.

In some aspects, the present disclosure provides an isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VH amino acid sequence comprises one or more amino acid change selected from 154F, I54Y, S59D, S59E, V101I, and A103S compared to SEQ ID NO: 3.

In some aspects, the present disclosure provides an isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VL amino acid sequence comprises one or more amino acid change selected from S52D, S52E, N92F, N92Y, Y94D. and Y94E compared to SEQ ID NO: 4.

Also provided is an isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-027 (i.e., an antibody according to Antibody Structure ID Number UGX126).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-066 (i.e., an antibody according to Antibody Structure ID Number UGX144).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-067 (i.e., an antibody according to Antibody Structure ID Number UGX145).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-028 and a light chain variable region (VL) according to VL-001 (i.e., an antibody according to Antibody Structure ID Number UGX156).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-029 and a light chain variable region (VL) according to VL-001 (i.e., an antibody according to Antibody Structure ID Number UGX157).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-061 and a light chain variable region (VL) according to VL-001 (i.e., an antibody according to Antibody Structure ID Number UGX175).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-055 and a light chain variable region (VL) according to VL-001 (i.e., an antibody according to Antibody Structure ID Number UGX182).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-050 (i.e., an antibody according to Antibody Structure ID Number UGX201).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-054 (i.e., an antibody according to Antibody Structure ID Number UGX202).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-027 (i.e., an antibody according to Antibody Structure ID Number UGX203).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-030 (i.e., an antibody according to Antibody Structure ID Number UGX204).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-066 (i.e., an antibody according to Antibody Structure ID Number UGX205).

Also provided is an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-067 (i.e., an antibody according to Antibody Structure ID Number UGX206).

In another aspect, the disclosure provides an isolated antibody or a fragment thereof that specifically binds to a human fibroblast growth factor 23 (FGF23) polypeptide, wherein the antibody or fragment thereof comprises a heavy chain (HC) amino acid sequence according to SEQ ID NO: 1 and a light chain (LC) amino acid sequence according to SEQ ID NO: 2, wherein the antibody or fragment thereof comprises one or more HC amino acid change relative to SEQ ID NO: 1 and/or one or more LC amino acid change relative to SEQ ID NO: 2, wherein the one or more HC amino acid change is selected from T28Y, T30F, T30K, N31D, N31E, N31F, N31Q, N31R, H32K, H32Y, Y33D, Y33L, Y33R, N52E, N52F, N52Q, N52R, N52Y, I54A, I54D, I54F, I54G, I54H, I54K, I54L, I54M, I54N, I54P, I54R, I54V, I54W, I54Y, S55E, S55F, S55I, S55Q, S55R, S55Y, S57E, S57F, S57R, S57Y, T58F, T58R, S59A, S59D, S59E, S59F, S59G, S59H, S59I, S59K, S59L, S59M, S59N, S59P, S59Q, S59R, S59T, S59V, S59W, S59Y, N60E, N60R, N60Y, A61F, A61R, A61Y, Q62L, Q62R, Q62Y, Q65E, Q65R, Q65Y, D99F, D99R, D99Y, I100D, I100E, I100F, I100Y, V101A, V101D, V101E, V101F, V101G, V101H, V101I, V101K, V101L, V101M, V101N, V101P, V101Q, V101R, V101S, V101W, D102E, D102F, D102K, D102R, D102Y, A103D, A103F, A103G, A103H, A103I, A103K, A103L, A103M, A103N, A103P, A103R, A103S, A103T, A103V, and A103W, and the one or more LC amino acid change is selected from Q27F, Q27R, G28D, G28Q, G28R, G28Y, I29F, I29L, S30D, S30E, S30F, S30L, S30Q, S30R, S30Y, S31D, S31E, S31F, S31Q, S31Y, A32F, A32V, V34F, V34I, D50E, D50F, D50L, D50Q, D50R, D50S, D50T, D50V, D50Y, A51V, S52A, S52D, S52E, S52F, S52G, S52H, S52I, S52K, S52L, S52M, S52N, S52P. S52Q, S52R, S52V, S52W, S52Y, S53D, S53E, S53F, S53R, S53Y, L54E, L54Y, E55R, E55Y, S67E, S67R, S67Y, T69R, T69Y, F91E, F91R, F91Y, N92A, N92D, N92E, N92F, N92G, N92H, N92I, N92K, N92L, N92M, N92P, N92Q, N92R, N92S, N92T, N92V, N92W, N92Y, D93F, D93Q, D93R, D93S, D93T, D93Y, Y94A, Y94D, Y94E, Y94F, Y94G, Y94H, Y94I, Y94K, Y94L, Y94M, Y94N, Y94P, Y94Q, Y94R, Y94S, Y94V, and Y94W.

In another aspect, the disclosure provides an isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 and a light chain variable region (VL) amino acid sequence according to SEQ ID NO: 4, wherein the antibody or fragment thereof comprises one or more VH amino acid change relative to SEQ ID NO: 3 and/or one or more VL amino acid change relative to SEQ ID NO: 4, wherein the one or more VH amino acid change is selected from T28Y, T30F, T30K, N31D, N31E, N31F, N31Q, N31R, H32K, H32Y, Y33D, Y33L, Y33R, N52E, N52F, N52Q, N52R, N52Y, I54A, I54D, I54F, I54G, I54H, I54K, I54L, I54M, I54N, I54P, I54R, I54V, I54W, I54Y, S55E, S55F, S55I, S55Q, S55R, S55Y, S57E, S57F, S57R, S57Y, T58F, T58R, S59A, S59D, S59E, S59F, S59G, S59H, S59I, S59K, S59L, S59M, S59N, S59P, S59Q, S59R, S59T, S59V, S59W, S59Y, N60E, N60R, N60Y, A61F, A61R, A61Y, Q62L, Q62R, Q62Y, Q65E, Q65R, Q65Y, D99F, D99R, D99Y, I100D, I100E, I100F, I100Y, V101A, V101D, V101E, V101F, V101G, V101H, V101I, V101K, V101L, V101M, V101N, V101P, V101Q, V101R, V101S, V101W, D102E, D102F, D102K, D102R, D102Y, A103D, A103F, A103G, A103H, A103I, A103K, A103L, A103M, A103N, A103P, A103R, A103S, A103T, A103V, and A103W, and the one or more VL amino acid change is selected from Q27F, Q27R, G28D, G28Q, G28R, G28Y, 129F, 129L, S30D, S30E, S30F, S30L, S30Q, S30R, S30Y, S31D, S31E, S31F, S31Q, S31Y, A32F, A32V, V34F, V34I, D50E, D50F, D50L, D50Q, D50R, D50S, D50T, D50V, D50Y, A51V, S52A, S52D, S52E, S52F, S52G, S52H, S52I, S52K, S52L, S52M, S52N, S52P, S52Q, S52R, S52V, S52W, S52Y, S53D, S53E, S53F, S53R, S53Y, L54E, L54Y, E55R, E55Y, S67E, S67R, S67Y, T69R, T69Y, F91E, F91R, F91Y, N92A, N92D, N92E, N92F, N92G, N92H, N92I, N92K, N92L, N92M, N92P, N92Q, N92R, N92S, N92T, N92V, N92W, N92Y, D93F, D93Q, D93R, D93S, D93T, D93Y, Y94A, Y94D, Y94E, Y94F, Y94G, Y94H, Y94I, Y94K, Y94L, Y94M, Y94N, Y94P, Y94Q, Y94R, Y94S, Y94V, and Y94W.

Also provided herein are FGF23-binding antibodies or fragments thereof having increased affinity for human FGF23 as compared to burosumab.

For example, the isolated antibody or fragment thereof may have about 2-fold to about 10-fold increased binding affinity for human FGF23 compared to burosumab.

In some embodiments, the isolated antibody or fragment thereof has a binding affinity to human FGF23 (K_{D}) of between about 1×10⁻¹² M to about 7×10⁻¹² M.

In some embodiments, the isolated antibodies or fragments provided herein are for use in a method of decreasing serum FGF23 in a subject in need thereof.

In some embodiments, the isolated antibodies or fragments provided herein are for use in a method of increasing renal maximum threshold for phosphate reabsorption (TmP) in a subject in need thereof.

In some embodiments, the isolated antibodies or fragments provided herein are for use in a method of increasing serum inorganic phosphorus (Pi) in a subject in need thereof.

In some embodiments, the isolated antibodies or fragments provided herein are for use in a method of increasing serum 1,25 dihy droxyvitamin D (1,25[OH]2D) concentration in a subject in need thereof.

In some embodiments, the isolated antibodies or fragments provided herein are for use in a method of treating a hypophosphatemic condition in subject in need thereof.

In some embodiments, the subject is diagnosed with X-linked hypophosphatemia (XLH) or tumor-induced osteomalacia (TIO).

Also provided herein are methods of decreasing serum FGF23 in a subject in need thereof, comprising administering an isolated antibody or fragment thereof described herein to the subject.

In another aspect, the disclosure provides methods of increasing renal maximum threshold for phosphate reabsorption (TmP) in a subject in need thereof, comprising administering an isolated antibody or fragment thereof described herein to the subject.

Also provided are methods of increasing serum inorganic phosphorus (Pi) in a subject in need thereof, comprising administering an isolated antibody or fragment thereof described herein to the subject.

Also provided are methods of increasing serum 1,25 dihydroxyvitamin D (1,25[OH]2D) concentration in a subject in need thereof, comprising administering an isolated antibody or fragment thereof described herein to the subject.

Also provided are methods of treating a hypophosphatemic condition in subject in need thereof, comprising administering an isolated antibody or fragment thereof described herein to the subject.

In some embodiments, the subject to be treated according to the methods described herein is diagnosed with X-linked hypophosphatemia (XLH) or tumor-induced osteomalacia (TIO).

These and other aspects and features of the disclosure are described in the following sections of the application.

### BRIEF DESCRIPTION OF THE DRAWINGS

The foregoing and other objects, features and advantages of the presently disclosed systems and methods will become apparent from the following description of preferred embodiments, as illustrated in the accompanying drawings. Like-referenced elements identify common features in the corresponding drawings.

FIG. 1 is a chart showing percent deuteration measured in HDX-MS analysis of FGF23 in the presence of burosumab. Three boxed regions identify amino acid residues potentially implicated in antibody binding as evidenced by increased protection from deuteration.

FIG. 2 is a depiction of protected region 1, protected region 2, and protected region 3 as defined herein by HDX-MS analysis superimposed on the 2P39 crystal structure of FGF23.

FIG. 3 is a chart showing percent deuteration measured in HDX-MS analysis of burosumab in the presence of FGF23. Six regions of low deuteration were identified, implicating amino acid residues involved in antigen binding.

FIG. 4 shows crystallographic data collection and refinement statistics for a FGF23(R179Q):burosumab Fab complex determined by molecular replacement methods using available structures (2P39.PDB and 2VEN.PDB).

FIG. 5 is two molecules of 3A crystal structure of the hFGF23(R179Q):burosumab Fab complex in the asymmetric unit. The two complex molecules are held together by FGF23 dimer formed by residues known to be involved in FGFR1c binding.

FIG. 6 is a ribbon model of the FGF23:Fab complex (only variable regions shown) with FGF23 epitope residues (C-alpha atoms) identified as colored purple spheres and FGF23-dimer/FGFR1c-binding residues (C-alpha atoms) as colored cyan spheres, burosumab light chain paratope residues as colored green spheres and burosumab heavy chain paratope residues as colored yellow spheres.

FIG. 7 shows previously reported epitopes identified in relevant systems (Yamazaki et al. JBMR. 2008 and Kanhasut et al. Sci Report. 2022) mapped onto the FGF23:burosumab crystal structure to illustrate that these epitopes differ from the epitopes identified in the present study (FIG. 6).

FIG. 8 shows the FGF23:Fab complex in ribbon model (left) and surface models (right panels), with FGF23 and burosumab binding interfaces rotated into view and detailed amino acid positions superimposed as stick models (right-most panels).

FIG. 9 shows FGF23 surface model interface with burosumab shown in ribbon and stick model. Panel A shows the FGF23:Fab complex with rotation of the FGF23 portion 90 degrees to display all the burosumab residues in contact with FGF23 at the binding interface. Panel B shows surface model of FGF23 (top) with burosumab hydrogen-bonding residues in stick model (superimposed and bottom). Panel C and Panel D show closer views of ionic interactions and aromatic stacking interactions at the FGF23:Fab interface, respectively. Black arrows indicate examples of burosumab residues targeted for structure-based rational design for affinity optimization.

FIG. 10 illustrates relevant interface positions of the burosumab light chain and human FGF23 based on crystal structure of the FGF23:Fab complex.

FIG. 11 illustrates relevant interface positions of the burosumab heavy chain and human FGF23 based on crystal structure of the FGF23:Fab complex.

FIG. 12 shows a ribbon model of the FGF23:Fab complex as shown in FIG. 6 (left-most column), highlighting certain amino acid substitutions hypothesized to improve affinity of variant antibodies compared to parental burosumab. Burosumab light chain residues are shown in green and heavy chain residues in yellow. FGF23 residues are shown in purple. Stick model interfaces between burosumab and FGF23, focusing on select paratope residues which, when modified alone or in combination, resulted in increased affinity for FGF23 as measured by SPR. Potential newly formed interactions that these modified residues form with nearby FGF23 epitope residues are described in each box.

### DETAILED DESCRIPTION

The present disclosure provides human FGF23-binding antibodies with increased affinity for human FGF23 as compared to burosumab. The antibodies of the disclosure may allow for reduced dosing frequency, increased convenience and patient compliance, and improved potency compared to burosumab while maintaining an acceptable toxicity and safety profile. To improve the affinity of burosumab for human FGF23, antibody variants were rationally designed to have increased human FGF23 binding affinity compared to burosumab.

### I. Definitions

Throughout the present specification, numerical ranges are provided for certain quantities. It is to be understood that these ranges comprise endpoints and all subranges therein, including each integer in and between a disclosed range. Thus, the range “from 50 to 80” includes all possible ranges therein (e.g., 51-79, 52-78, 53-77, 54-76, 55-75, 60-70, etc.) as well as each individual integer from 50 to 80 (e.g., 50, 51, 52, 53, 54, etc.). Where ranges are provided in the form of fractions, percentages, decimals, and the like, such ranges likewise include all possible subranges therein and each individual fraction, percentage, decimal, etc. in and between the disclosed range. For example, the range “from 0.1 to 1.0” includes all possible ranges therein (e.g., 0.2 to 0.9, etc.) and each individual 1/10th decimal from 0.1 to 1.0 (e.g., 0.1, 0.2, 0.3, 0.4, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”). “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

The term “affinity” (or “binding affinity”) refers to the strength of binding of a molecule or complex of molecules to a binding partner, such as a ligand or an epitope or an antigen. Affinity may be expressed in terms of the dissociation constant (K_{D}) which describes the rate of dissociation of a molecule or complex and its binding partner at equilibrium. K_{D} can be expressed as a ratio of a ligand's association rate constant (kₒₙ) and dissociation rate constant (k_{off}), or K_{D}=k_{off}/kₒₙ. With respect to antibody/antigen binding affinities, the rate of antibody/antigen complex formation at equilibrium is equal to the rate of dissociation of the complex to unbound antibody and antigen, and the affinity of the antibody can be expressed as 1/K_{D}, such that smaller K_{D} values reflect greater affinity of the antibody for its target antigen. Methods for determining binding affinity are known and include, for example, enzyme-linked immunosorbent assay (ELISA) approaches and surface plasmon resonance (SPR) based approaches.

The term “Antibody Structural Numbering System” or “ASN #” as used herein refers to a numbering scheme for the structural alignment and classification of full sequences from antibodies and antibody-like structures. A description of the ASN #system is provided at www.just-evotecbiologics.com/white-papers-application-notes/coreab-sequence-classification/(last visited Jul. 5, 2023; incorporated herein by reference in its entirety). The ASN #system is based on and is substantially identical to Honneger's numbering scheme, i.e., the “AHo” numbering scheme (see, e.g., Honegger, Annemarie. and Andreas PluÈckthun. “Yet another numbering scheme for immunoglobulin variable domains: an automatic modeling and analysis tool.” Journal of molecular biology 309.3 (2001): 657-670; incorporated herein by reference in its entirety), which “numbers the variable domains of the immunoglobulin superfamily in a homogenized format . . . based on structural alignments of the 3D structures of the immunoglobulin variable regions covering the observed length variation” (Dondelinger, Mathieu, et al. “Understanding the significance and implications of antibody numbering and antigen-binding surface/residue definition.” Frontiers in immunology 9 (2018): 2278).

The verb “comprise” as used in this description and in the claims and its conjugations are used in its non-limiting sense to mean that items following the word are included, but items not specifically mentioned are not excluded.

The term “treating” or “treatment” refers to one or more of relieving, alleviating, delaying, reducing, improving, or managing at least one symptom of a condition in a subject. Treating may also mean one or more of arresting, delaying the onset of (i.e., the period prior to clinical manifestation of the condition) or reducing the risk of developing or worsening a condition.

The term “subject” can refer to a human, non-human primate, mammal, rat, mouse, cow, horse, pig, sheep, goat, dog, cat and the like. The subject can be suspected of having or being at risk for having a hypophosphatemic condition such as XLH or TIO, or can be diagnosed with a hypophosphatemic condition such as XLH or TIO.

The present disclosure includes information that may be useful in understanding the presently disclosed subject matter. It is not an admission that any of the information provided herein is prior art or relevant to the presently claimed inventions, or that any publication specifically or implicitly referenced is prior art.

### II. Overview

Burosumab therapy effectively improves clinical symptoms of hypophosphatemia. A number of clinical trials have established burosumab as a safe and effective therapy for adults and children with X-linked hypophosphatemia (XLH) (Schindeler, et al. “Clinical evidence for the benefits of burosumab therapy for X-linked hypophosphatemia (XLH) and other conditions in adults and children.” Frontiers in Endocrinology 11 (2020): 338.). Additional studies have confirmed safe and effective treatment of tumor-induced osteomalacia (TIO) with burosumab (Jan de Beur, et al. “Burosumab for the treatment of tumor-induced osteomalacia.” Journal of Bone and Mineral Research 36.4 (2021): 627-635.).

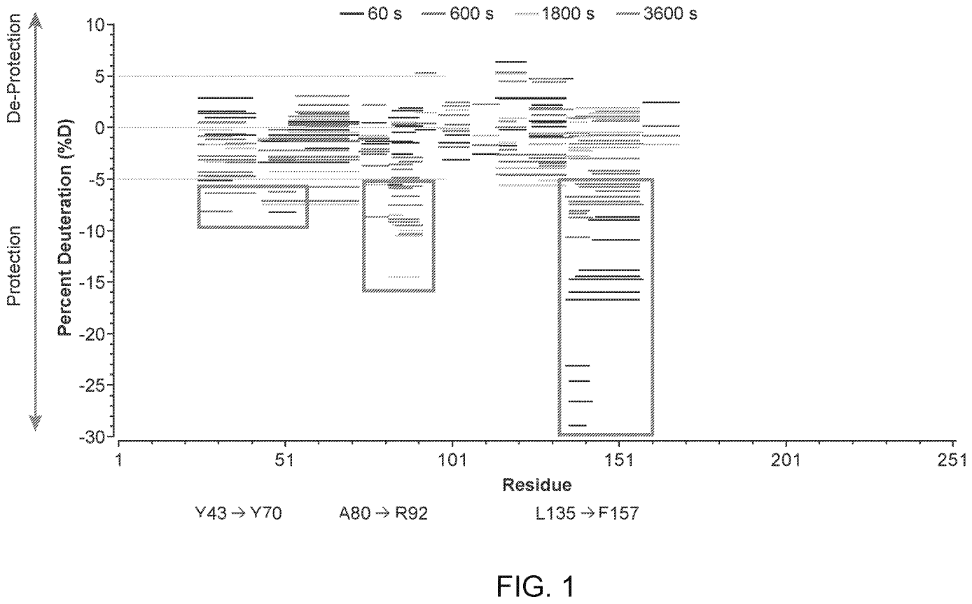

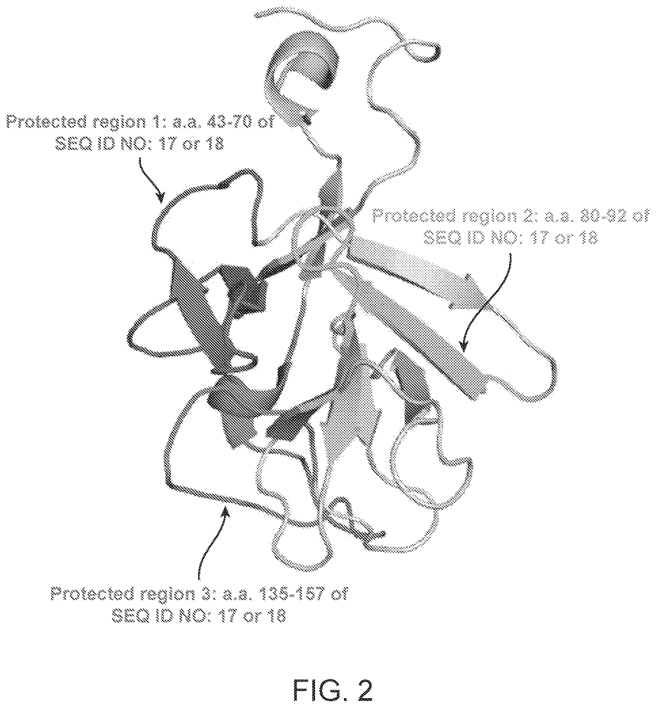

However, treatment with burosumab only returns serum phosphorous levels to 3.3 mg/dL after 64 weeks, which is near the 3.2 mg/dL lower limit of normal levels seen in healthy populations (see. e.g., Section 12, FIG. 1 and FIG. 2 of Summary Report of Benefit-Risk Assessment from the Health Sciences Authority of Singapore in connection with a New Drug Application for Crysvita, available at www.hsa.gov.sg/docs/default-source/hprg-tpb/summary-reports/crysvita_summary-report_2021.pdf). Further, treatment with burosumab requires subcutaneous injection every 2 weeks (Q2W) in pediatric patients and both adults and pediatric patients must undergo high dosing volumes of burosumab injection, with a maximum of up to 3 ml administered in three separate 1 ml injections, representing a significant treatment burden for patients.

Therefore, the present description provides human FGF23-binding antibodies having improved binding affinity to human FGF23 compared to burosumab. It is contemplated that improving the binding affinity of burosumab will increase the potency of the antibody, reduce the amount of bioavailable FGF23 in serum, and enable reductions in dosing amounts and/or dosing frequency, thereby lessening the treatment burden of patients in need of human FGF23-binding antibody therapy, without significantly negatively affecting the safety profile of the therapy.

Burosumab is believed to bind to intact human FGF23 with high affinity. However, different assays for measuring burosumab affinity utilizing varying source materials have yielded different values. For instance, the Assessment Report for Crysvita from the European Medicines Agency reports: “In a Biacore competitive binding study [burosumab] was shown to bind with similar binding affinity (K_{D}~10-11 M) to human, cynomolgus monkey and rabbit FGF23” (see Assessment report: Crysvita, 14 Dec. 2017 (EMA/148319/2018), available at www.ema.europa.eu/en/documents/assessment-report/crysvita-epar-public-assessment-report_en.pdf). Studies by the present inventors used a surface plasmon resonance (SPR) approach to determine burosumab affinity using recombinant human FGF23 (R179Q) from HEK293S cell supernatant and burosumab manufactured using CHO cells and found an estimated binding affinity (K_{D}) on the order of approximately 1×10⁻¹¹ to 3×10⁻¹¹ (data not shown). Yamazaki et al. reported an affinity of 4.7×10⁻¹¹ for FN1, a mouse monoclonal anti-FGF23 antibody described by the authors as binding human FGF23 in like manner as burosumab (Yamazaki, Yuji, et al. “Anti-FGF23 neutralizing antibodies show the physiological role and structural features of FGF23.” Journal of Bone and Mineral Research 23.9 (2008): 1509-1518.). Another group utilized ELISA to determine burosumab affinity using recombinant human FGF23 expressed in E. coli and burosumab expressed in HEK293 cells and found an estimated binding affinity of approximately 0.5×10⁻⁹ or 0.77×10⁻⁹ (Kanhasut, et al. “Prediction of the structural interface between fibroblast growth factor 23 and burosumab using alanine scanning and molecular docking”. 16 May 2022, PREPRINT (Version 1) available at Research Square [doi.org/10.21203/rs.3.rs-1630525/v1]). The disparate results from studies evaluating burosumab affinity underscore the need for a comprehensive study of the binding kinetics of this monoclonal antibody to human FGF23.

Kanhasut et al. further investigated approaches to improving burosumab binding affinity to FGF23. Using alanine scanning data and molecular docking to model the interaction between a homology-modeled burosumab and a crystal structure of FGF23, the authors identified four amino acid changes on the burosumab light chain variable domain (VL) that were predicted to enhance interactions with the epitope: A32S, S52D, S67Y, and T69D (Kabat numbering scheme). Based on original alanine scanning data, the authors also interrogated a fifth change, V97A (Kabat numbering scheme) on the heavy chain variable domain (VH). This study provided data supporting an approximate 3-fold enhancement in FGF23-binding affinity of K_{D}=0.21×10⁹ in a burosumab variant antibody having both amino acid changes VH:V97A (i.e., V101A per the linear numbering scheme) and VL:A32S (i.e., A32S per both Kabat and linear numbering schemes).

The Kanhasut et al. study, however, has several deficits that call into question the validity and biological significance of its conclusions. For instance, Kanhasut et al. employed FGF23 expressed in E. coli cells, meaning the antigenic protein was not glycosylated in a form similar to that which would be found in a mammalian cell-based system. Further, for K_{D} determination, Kanhasut et al. utilized a titration ELISA approach, which is recognized as being highly variable and not the most precise assay for affinity measurement. Inconsistencies in the K_{D} of burosumab reported throughout Kanhasut et al. belie the high degree of variability in the assay and call into question the ultimate accuracy and significance of the study. Last, structural evaluation of the burosumab:FGF23 complex described herein suggests that the epitope residues identified by Kanhasut et al. do not agree with the epitope residues identified in the crystal structure. In fact, the present inventors conducted surface plasmon resonance analysis of the VH:V97A and VL:A32S (i.e., heavy chain V101A and light chain A32S per the linear numbering scheme, respectively) amino acid substitutions and demonstrated that these changes impair rather than improve affinity of burosumab (data not shown).

These and other studies reveal that more definitive investigation of burosumab binding kinetics, including K_{D} determination of burosumab binding to human FGF23, and precise epitope mapping is needed along with new approaches for improving the binding affinity of burosumab. Importantly, in designing antibodies with improved binding affinity compared to burosumab, it is critical to fine-tune the affinity to human FGF23 such that potency is improved without compromising safety. For instance, increasing affinity too much may result in decreases in serum FGF23 concentrations in subjects that lead to unsafe serum phosphorous levels and symptoms of hvperphosphatemia. On the other hand, very small improvements in affinity may not provide clinical benefit compared to treatment with burosumab. Accordingly, rationally designed anti-human FGF23 antibodies are needed having binding affinity fine-tuned to increase potency without leading to unsafe levels of serum phosphorous or clinical symptoms of hyperphosphatemia.

The present disclosure provides rationally designed human FGF23-binding antibodies with increased affinity for human FGF23 as compared to burosumab. Rational design of the human FGF23-binding antibodies described herein may involve, for example, crystallographic analysis of the burosumab-human FGF23 interaction and/or in silico modeling involving any of conformational sampling using molecular dynamics and choice of an ensemble of target conformational states representing docking templates; generation of a homology model of lead antibody/ies and energy minimization, selecting probable conformational states; guided molecular docking of lead antibody models onto the FGFR23 structure (e.g., epitope selected based on HDX data); paratope and epitope determinations based on docking results; evaluation and ranking of antigen-antibody binding interactions (e.g. 1st shell paratope:epitope and 2nd shell); comparison to recently published results describing burosumab:FGF23 binding (Kanhasut et al., Nature (2022) 12:14754); assessment of any additional changes needed (i.e., stability, developability); and/or assessment of post-translational modifications and associated remediations. From these studies, individual or multiple amino acid changes can be introduced into the burosumab backbone sequence and analyzed for binding properties. Variant antibody designs can be screened to identify burosumab variants with improved binding properties.

Thus, the present disclosure provides human FGF23-binding antibodies with increased affinity for human FGF23 as compared to burosumab, enabling reduced dosing frequency, increased convenience and patient compliance, and improved potency compared to burosumab while maintaining an acceptable toxicity and safety profile.

To improve the affinity of burosumab for human FGF23, antibody variants were designed to have increased human FGF23 binding affinity compared to burosumab.

These and other aspects of the antibodies of the disclosure and uses thereof are further described below.

### III. Compositions

The compositions of the present disclosure comprise one or more antibodies or antigen-binding fragments thereof that bind to human FGF23. In some embodiments, the human FGF23-binding antibodies are variants of burosumab engineered to have improved affinity (i.e., engineered human FGF23-binding antibodies).

In some embodiments, the engineered human FGF23-binding antibodies provided herein can be administered to subjects for use in decreasing serum FGF23 in the subjects.

In some embodiments, the engineered human FGF23-binding antibodies provided herein can be administered to subjects for use in increasing renal maximum threshold for phosphate reabsorption (TmP) in the subjects.

In some embodiments, the engineered human FGF23-binding antibodies provided herein can be administered to subjects for use in increasing serum inorganic phosphorus (Pi) in the subjects.

In some embodiments, the engineered human FGF23-binding antibodies provided herein can be administered to subjects for use in increasing serum 1,25 dihydroxyvitamin D (1,25[OH]2D) concentration in the subjects.

In some embodiments, the engineered human FGF23-binding antibodies provided herein are useful in the treatment of hypophosphatemic conditions in subjects including XLH or osteomalacia, such as tumor-induced osteomalacia (TIO).

In some embodiments, the engineered human FGF23-binding antibodies provided herein have increased affinity for human FGF23 as compared to burosumab.

In some embodiments, the binding affinity of the antibodies provided herein to human FGF23 is increased relative to burosumab by decreasing the K_{D} of the antibodies to between about 1×10⁻² and about 7×10⁻¹².

In some embodiments, the binding affinity of the antibodies provided herein to human FGF23 is increased about 2-fold to about 10-fold compared to burosumab. For instance, the binding affinity of the antibodies provided herein to human FGF23 may be increased by about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold compared to burosumab.

In some embodiments, the increased binding affinity of the antibodies provided herein to human FGF23 relative to burosumab results in a decrease in free FGF23 in subjects relative to the decrease observed with burosumab treatment in subjects. Free FGF23 refers to bioavailable FGF23. Binding of FGF23 by the antibodies provided herein can reduce the amount of free FGF23 in subjects by at least about 5% or by at least about 10% compared to burosumab.

The engineered human FGF23-binding antibodies described herein may have one or more amino acid substitution compared to the amino acid sequence of burosumab. For instance, the engineered FGF23-binding antibodies described herein may comprise one or more amino acid substitutions in the heavy chain sequence relative to the heavy chain sequence of burosumab (SEQ ID NO: 1). The engineered FGF23-binding antibodies described herein may comprise one or more amino acid substitutions in the light chain sequence relative to the light chain sequence of burosumab (SEQ ID NO: 2). The engineered FGF23-binding antibodies described herein may comprise one or more amino acid substitutions in the heavy chain sequence relative to the heavy chain sequence of burosumab (SEQ ID NO: 1) and one or more amino acid substitutions in the light chain sequence relative to the light chain sequence of burosumab (SEQ ID NO: 2).

The engineered human FGF23-binding antibodies described herein may have 1, 2, 3, 4, 5, 6, 7, 8, 9, 10, 11, 12, 13, 14, 15, 16, 17, 18, 19, 20, or more than 20 amino acid substitutions compared to the amino acid sequence of burosumab. For instance, the engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid substitutions in the heavy chain sequence relative to the heavy chain sequence of burosumab (SEQ ID NO: 1). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid substitutions in the light chain sequence relative to the light chain sequence of burosumab (SEQ ID NO: 2). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid substitutions in the heavy chain sequence relative to the heavy chain sequence of burosumab (SEQ ID NO: 1) and 1, 2, 3, 4, 5, 6, 7, 8, 9, 10, 11, 12, 13, 14, 15, 16, 17, 18, 19, 20, or more than 20 amino acid substitutions in the light chain sequence relative to the light chain sequence of burosumab (SEQ ID NO: 2).

The engineered human FGF23-binding antibodies described herein may have 1, 2, 3, 4, 5, 6, 7, 8, 9, 10, 11, 12, 13, 14, 15, 16, 17, 18, 19, 20, or more than 20 amino acid deletions compared to the amino acid sequence of burosumab. For instance, the engineered FGF23 -binding antibodies described herein may comprise 1, 2, 3, 4, 5, 6, 7, 8, 9, 10, 11, 12, 13, 14, 15, 16, 17, 18, 19, 20, or more than 20 amino acid deletions in the heavy chain sequence relative to the heavy chain sequence of burosumab (SEQ ID NO: 1). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid deletions in the light chain sequence relative to the light chain sequence of burosumab (SEQ ID NO: 2). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid deletions in the heavy chain sequence relative to the heavy chain sequence of burosumab (SEQ ID NO: 1) and 1, 2, 3, 4, 5, 6, 7, 8, 9, 10, 11, 12, 13, 14, 15, 16, 17, 18, 19, 20, or more than 20 amino acid deletions in the light chain sequence relative to the light chain sequence of burosumab (SEQ ID NO: 2).

The engineered human FGF23-binding antibodies described herein may have 1, 2, 3, 4, 5, 6, 7, 8, 9, 10, 11, 12, 13, 14, 15, 16, 17, 18, 19, 20, or more than 20 amino acid insertions compared to the amino acid sequence of burosumab. For instance, the engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid insertions in the heavy chain sequence relative to the heavy chain sequence of burosumab (SEQ ID NO: 1). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid insertions in the light chain sequence relative to the light chain sequence of burosumab (SEQ ID NO: 2). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid insertions in the heavy chain sequence relative to the heavy chain sequence of burosumab (SEQ ID NO: 1) and 1, 2, 3, 4, 5, 6, 7, 8, 9, 10, 11, 12, 13, 14, 15, 16, 17, 18, 19, 20, or more than 20 amino acid insertions in the light chain sequence relative to the light chain sequence of burosumab (SEQ ID NO: 2).

The engineered human FGF23-binding antibodies described herein may have 1, 2, 3, 4, 5, 6, 7, 8, 9, 10, 11, 12, 13, 14, 15, 16, 17, 18, 19, 20, or more than 20 amino acid changes compared to the amino acid sequence of burosumab, wherein the changes comprise a combination of substitutions, deletions, and/or insertions relative to the amino acid sequence of burosumab. For instance, the engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid changes in the heavy chain sequence relative to the heavy chain sequence of burosumab (SEQ ID NO: 1), wherein the changes comprise a combination of substitutions, deletions, and/or insertions relative to the heavy chain sequence of burosumab (SEQ ID NO: 1). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid changes in the light chain sequence relative to the light chain sequence of burosumab (SEQ ID NO: 2), wherein the changes comprise a combination of substitutions, deletions, and/or insertions relative to the light chain sequence of burosumab (SEQ ID NO: 2). The engineered FGF23-binding antibodies described herein may comprise 1, 2, 3, 4, 5, 6, 7, 8, 9, 10, 11, 12, 13, 14, 15, 16, 17, 18, 19, 20, or more than 20 amino acid changes in the heavy chain sequence relative to the heavy chain sequence of burosumab (SEQ ID NO: 1) and 1, 2, 3, 4, 5, 6, 7, 8, 9, 10, 11, 12, 13, 14, 15, 16, 17, 18, 19, 20, or more than 20 amino acid changes in the light chain sequence relative to the light chain sequence of burosumab (SEQ ID NO: 2), wherein the changes comprise a combination of substitutions, deletions, and/or insertions relative to the heavy chain and light chain sequences of burosumab (SEQ ID NO: 1 and SEQ ID NO: 2, respectively).

In some embodiments, the engineered human FGF23-binding antibodies described herein comprise one or more amino acid substitutions, deletions, and/or insertions (collectively, “changes”) relative to the amino acid sequence of the burosumab heavy chain sequence and/or burosumab light chain sequence, wherein the one or more amino acid changes affect one or more burosumab paratope residue and/or one or more residues neighboring a burosumab paratope residue. Example burosumab paratope regions and paratope residues are provided in Table 4 herein. Burosumab residues neighboring a burosumab paratope residue may include residues within 1, within 2, within 3, within 4, within 5, within 6, within 7, within 8, within 9, or within 10 amino acid residues of a burosumab paratope residue.

In some embodiments, the engineered human FGF23-binding antibodies described herein comprise one or more light chain variable region amino acid changes provided in Table 13.

In some embodiments, the engineered human FGF23-binding antibodies described herein comprise one or more heavy chain variable region amino acid changes provided in Table 14.

In some embodiments, the engineered human FGF23-binding antibodies described herein comprise two amino acid changes according to those provided in Table 15.

In some embodiments, an engineered human FGF23-binding antibody provided herein is one of UGX101 through UGX196 described in Table 16 and Table 17.

Amino Acid Changes of Certain Engineered FGF23-binding Antibodies

The engineered FGF23-binding antibodies described herein may comprise one or more amino acid change in the heavy chain variable region (VH) relative to SEQ ID NO: 3, wherein the one or more amino acid change is selected from T30E, T30F, T30Q, T30S, T30Y, N31D, N31E, N31F, N31Q, N31R, N31Y, H32E, H32F, H32Q, H32S, H32T, I50D, 150E, I50F, 150S, I50T, I50Y, N52E, N52F, N52Q, N52Y, I54E, I54F, I54L, I54Q, I54S, I54T, I54V, I54Y, S55E, S55F, S55Q, S55T, S55Y, S59D, S59E, S59F, S59I, S59L, S59Q, S59T, S59V, S59Y, D99E, D99Q, D99Y, I100D, I100E, I100F, I100L, I100Q, I100V, I100Y, V101F, V101I, V101L, V101T, V101Y, D102E, D102F, D102I, D102L, D102Q, D102Y, A103D, A103E, A103F, A103Q, A103S, A103T, and A103Y.

The engineered FGF23-binding antibodies described herein may comprise one or more amino acid change in the light chain variable region (VL) relative to SEQ ID NO: 4, wherein the one or more amino acid change is selected from A25L, A25V, 129F, 129L, 129V, S30D, S30E, S30F, 5301, S30L, S30Q, S30T, S30V, S30Y, S31D, S31E, S31F, S31I, S31L, S31Q, S31T, S31V, S31Y, A32F, A32I, A32L, A32T, A32V, A32Y, V34F, V34I, V34L, V34Y, D50E, D50F, D50I, D50L, D50Q, D50R, D50S, D50T, D50V, A51F, A51I, A51L, A51S, A51T, A51V, S52D, S52E, S52F, S52Q, S52T, S52Y, S53D, S53E, S53F, S53I, S53L, S53Q, S53R, S53T, S53V, S53Y, Q90F, Q90Y, F91K, F91R, F91Y, N92D, N92E, N92F, N92Q, N92S, N92T, N92Y, D93E, D93F, D93Q, D93R, D93S, D93T, D93Y, Y94D, Y94E, Y94F, Y94Q, Y94S, and Y94T.

The engineered FGF23-binding antibodies described herein may comprise one or more amino acid change in the heavy chain variable region (VH) and the light chain variable region (VL), relative to SEQ ID NO: 3 and SEQ ID NO: 4, respectively, wherein the one or more amino acid change in the VH is selected from T30E, T30F, T30Q, T30S, T30Y, N31D, N31E, N31F, N31Q, N31R, N31Y, H32E, H32F, H32Q, H32S, H32T, I50D, 150E, I50F, I50S, I50T, I50Y, N52E, N52F, N52Q, N52Y, I54E, I54F, I54L, I54Q, I54S, I54T, I54V, I54Y, S55E, S55F, S55Q, S55T, S55Y, S59D, S59E, S59F, S59I, S59L, S59Q, S59T, S59V, S59Y, D99E, D99Q, D99Y, I100D, I100E, I100F, I100L, I100Q, I100V, I100Y, V11F, V101I, V100L, V101T, V101Y, D102E, D102F, D102I, D102L, D102Q, D102Y, A103D, A103E, A103F, A103Q, A103S, A103T, and A103Y, and wherein the one or more amino acid change in the VL is selected from A25L, A25V, 129F, 129L, I29V, S30D, S30E, S30F, 5301, S30L, S30Q, S30T, S30V, S30Y, S31D, S31E, S31F, S31I, S31L, S31Q, S31T, S31V, S31Y, A32F, A32I, A32L, A32T, A32V, A32Y, V34F, V34I, V34L, V34Y, D50E, D50F, D50I, D50L, D50Q, D50R, D50S, D50T, D50V, A51F, A51I, A51L, A51S, A51T, A51V, S52D, S52E, S52F, S52Q, S52T, S52Y, S53D, S53E, S53F, S53I, S53L, S53Q, S53R, S53T, S53V, S53Y, Q90F, Q90Y, F91K, F91R, F91Y, N92D, N92E, N92F, N92Q, N92S, N92T, N92Y, D93E, D93F, D93Q, D93R, D93S, D93T, D93Y, Y94D, Y94E, Y94F, Y94Q, Y94S, and Y94T.

The engineered FGF23-binding antibodies described herein may comprise at least two amino acid changes in the heavy chain variable region (VH) relative to SEQ ID NO: 3, in the light chain variable region (VL) relative to SEQ ID NO: 4, or in the VH relative to SEQ ID NO: 3 and in the VL relative to SEQ ID NO: 4, wherein the at least two amino acid changes are selected from VL:S31E/VL:D50F, VH:N31F/VH:S59F, VL:D50F/VL:S53E, VL:S31F/VL:S52E, VL:D50F/VH:V101I, VH:S59D/VH:V101I, VL:S31F/VL:S53E, VL:N92E/VL:D93F, VL:S31E/VL:S53E, VL:S31F/VL:D50F. VH:N31E/VH:S59E, VH:S57E/VH:S59D, VH:N31E/VH:I54F, VH:S59T/VH:V101L, VH:I54Y/VH:V101F, VH:N52F/VH:V101I, VL:N92D/VL:D93F, VH:N52F/VH:V101L, VL:S31F/VL:D50E, VL:Y94D/VH:S59D, VL:Y94D/VH:S59E, VL:N92F/VH:S59E, VL:N92Y/VH:S59E, VL:Y94E/VH:S59E, VL:S52D/VH:S59E. and VL:S52E/VH:S59E.

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VH amino acid sequence comprises one or more amino acid change selected from 154F, I54Y, S59D, S59E, V101I, and A103S compared to SEQ ID NO: 3.

21. An isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VL amino acid sequence comprises one or more amino acid change selected from S52D, S52E, N92F, N92Y, Y94D, and Y94E compared to SEQ ID NO: 4.

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-027 (UGX126).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-066 (UGX144).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-067 (UGX145).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-028 and a light chain variable region (VL) according to VL-001 (UGX156).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-029 and a light chain variable region (VL) according to VL-001 (UGX157).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-061 and a light chain variable region (VL) according to VL-001 (UGX175).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-055 and a light chain variable region (VL) according to VL-001 (UGX182).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-050 (UGX201).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-054 (UGX202).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-027 (UGX203).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-030 (UGX204).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-066 (UGX205).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-067 (UGX206).

In some embodiments, the engineered FGF23-binding antibodies described herein may comprise an isolated antibody or a fragment thereof that specifically binds to a human fibroblast growth factor 23 (FGF23) polypeptide, wherein the antibody or fragment thereof comprises a heavy chain (HC) amino acid sequence according to SEQ ID NO: 1 and a light chain (LC) amino acid sequence according to SEQ ID NO: 2, wherein the antibody or fragment thereof comprises one or more HC amino acid change relative to SEQ ID NO: 1 and/or one or more LC amino acid change relative to SEQ ID NO: 2, wherein the one or more HC amino acid change is selected from T28Y, T30F, T30K, N31D, N31E, N31F, N31Q, N31R, H32K, H32Y, Y33D, Y33L, Y33R, N52E, N52F, N52Q, N52R, N52Y, I54A, I54D, I54F, I54G, I54H, I54K, I54L, I54M, I54N, I54P, I54R, I54V, I54W, I54Y, S55E, S55F, 5551, S55Q, S55R, S55Y, S57E, S57F, S57R, S57Y, T58F, T58R, S59A, S59D, S59E, S59F, S59G, S59H, S59I, S59K, S59L, S59M, S59N, S59P, S59Q, S59R, S59T, S59V, S59W, S59Y, N60E, N60R, N60Y, A61F, A61R, A61Y. Q62L, Q62R, Q62Y, Q65E. Q65R, Q65Y, D99F, D99R, D99Y, I100D, I100E, I100F, I100Y, V101A, V101D, V101E, V101F, V101G, V101H, V101I, V101K, V101L, V101M, V101N, V101P, V101Q, V101R, V101S, V101W, D102E, D102F, D102K, D102R, D102Y, A103D, A103F, A103G, A103H, A103I, A103K, A103L, A103M, A103N, A103P, A103R, A103S, A103T, A103V. and A103W, and the one or more LC amino acid change is selected from Q27F, Q27R, G28D, G28Q, G28R, G28Y, 129F, 129L, S30D, S30E, S30F, S30L, S30Q, S30R, S30Y, S31D, S31E, S31F, S31Q, S31Y, A32F, A32V, V34F, V34I, D50E, D50F, D50L, D50Q, D50R, D50S, D50T, D50V, D50Y, A51V, S52A, S52D, S52E, S52F, S52G, S52H, S52I, S52K, S52L, S52M, S52N, S52P, S52Q, S52R, S52V, S52W, S52Y, S53D, S53E, S53F, S53R, S53Y, L54E. L54Y. E55R, E55Y, S67E, S67R, S67Y, T69R, T69Y, F91E, F91R, F91Y, N92A, N92D, N92E, N92F, N92G, N92H, N92I, N92K, N92L, N92M, N92P, N92Q, N92R, N92S, N92T, N92V, N92W, N92Y, D93F, D93Q, D93R, D93S, D93T, D93Y, Y94A, Y94D, Y94E, Y94F, Y94G, Y94H, Y94I, Y94K, Y94L, Y94M, Y94N, Y94P, Y94Q, Y94R, Y94S, Y94V, and Y94W.

36. An isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 and a light chain variable region (VL) amino acid sequence according to SEQ ID NO: 4, wherein the antibody or fragment thereof comprises one or more VH amino acid change relative to SEQ ID NO: 3 and/or one or more VL amino acid change relative to SEQ ID NO: 4, wherein the one or more VH amino acid change is selected from T28Y, T30F, T30K, N31D, N31E, N31F, N31Q, N31R, H32K, H32Y, Y33D, Y33L, Y33R, N52E, N52F, N52Q, N52R, N52Y, I54A, I54D, I54F, I54G, I54H, I54K, I54L, I54M, I54N, I54P, I54R, I54V, I54W, I54Y, S55E, S55F, S55I, S55Q, S55R, S55Y, S57E, S57F, S57R, S57Y, T58F, T58R, S59A, S59D, S59E, S59F, S59G, S59H, S59I, S59K, S59L, S59M, S59N, S59P, S59Q, S59R, S59T, S59V, S59W, S59Y, N60E, N60R, N60Y, A61F, A61R, A61Y, Q62L, Q62R, Q62Y, Q65E, Q65R, Q65Y, D99F, D99R, D99Y, I100D, I100E, I100F, I100Y, V101A, V101D, V101E, V101F, V101G, V101H, V101I, V101K, V101L, V101M, V101N, V101P, V101Q, V101R, V101S, V101W, D102E, D102F, D102K, D102R, D102Y, A103D, A103F, A103G, A103H, A103I, A103K, A103L, A103M, A103N, A103P, A103R, A103S, A103T, A103V, and A103W, and the one or more VL amino acid change is selected from Q27F, Q27R, G28D, G28Q, G28R, G28Y, I29F, I29L, S30D, S30E, S30F, S30L, S30Q, S30R, S30Y, S31D, S31E, S31F, S31Q, S31Y, A32F, A32V, V34F, V34I, D50E, D50F, D50L, D50Q, D50R, D50S, D50T, D50V, D50Y, A51V, S52A, S52D, S52E, S52F, S52G, S52H, S52I, S52K, S52L, S52M, S52N, S52P, S52Q, S52R, S52V, S52W, S52Y, S53D, S53E, S53F, S53R, S53Y, L54E, L54Y, E55R, E55Y, S67E, S67R, S67Y, T69R, T69Y, F91E, F91R, F91Y, N92A, N92D, N92E, N92F, N92G, N92H, N92I, N92K, N92L, N92M, N92P, N92Q, N92R, N92S, N92T, N92V, N92W, N92Y, D93F, D93Q, D93R, D93S, D93T, D93Y, Y94A, Y94D, Y94E, Y94F, Y94G, Y94H, Y94I, Y94K, Y94L, Y94M, Y94N, Y94P, Y94Q, Y94R, Y94S, Y94V, and Y94W.

Complementarity Determining Regions of Certain Engineered FGF23-binding Antibodies

Burosumab has complementarity-determining regions (CDRs) defined as follows: heavy chain CDR1 having amino acid sequence NHYMH (SEQ ID NO: 11), referred to herein as HCDR1-001; heavy chain CDR2 having amino acid sequence IINPISGSTSNAQKFQG (SEQ ID NO: 12), referred to herein as HCDR2-001: heavy chain CDR3 having amino acid sequence DIVDAFDF (SEQ ID NO: 13), referred to herein as HCDR3-001; light chain CDR1 having amino acid sequence RASQGISSALV (SEQ ID NO: 14), referred to herein as LCDR1-001; light chain CDR1 having amino acid sequence DASSLES (SEQ ID NO: 15), referred to herein as LCDR2-001; and light chain CDR1 having amino acid sequence QQFNDYFT (SEQ ID NO: 16), referred to herein as LCDR3-001.

The engineered FGF23-binding antibodies described herein may comprise one or more unique complementarity-determining regions (CDRs) relative to those of burosumab. That is, the engineered FGF23-binding antibodies may have one or more amino acid changes relative to SEQ ID NO: 11, SEQ ID NO: 12, SEQ ID NO: 13, SEQ ID NO: 14, SEQ ID NO: 15, and/or SEQ ID NO: 16.

In some embodiments, the disclosure provides an isolated antibody or a fragment thereof that specifically binds to a human fibroblast growth factor 23 (FGF23) polypeptide, wherein the antibody or fragment thereof comprises a heavy chain complementarity determining region 1 (HCDR1) according to an amino acid sequence selected from SEQ ID NO: 11 and SEQ ID NOs: 22-32.

In some embodiments, the disclosure provides an isolated antibody or a fragment thereof that specifically binds to a human fibroblast growth factor 23 (FGF23) polypeptide, wherein the antibody or fragment thereof comprises a heavy chain complementarity determining region 2 (HCDR2) according to an amino acid sequence selected from SEQ ID NO: 12 and SEQ ID NOs: 33-65.

In some embodiments, the disclosure provides an isolated antibody or a fragment thereof that specifically binds to a human fibroblast growth factor 23 (FGF23) polypeptide, wherein the antibody or fragment thereof comprises a heavy chain complementarity determining region 3 (HCDR3) according to an amino acid sequence selected from SEQ ID NO: 13 and SEQ ID NOs: 66-93.

In some embodiments, the disclosure provides an isolated antibody or a fragment thereof that specifically binds to a human fibroblast growth factor 23 (FGF23) polypeptide, wherein the antibody or fragment thereof comprises a light chain complementarity determining region 1 (LCDR1) according to an amino acid sequence selected from SEQ ID NO: 14 and SEQ ID NOs: 94-126.

In some embodiments, the disclosure provides an isolated antibody or a fragment thereof that specifically binds to a human fibroblast growth factor 23 (FGF23) polypeptide, wherein the antibody or fragment thereof comprises a light chain complementarity determining region 2 (LCDR2) according to an amino acid sequence selected from SEQ ID NO: 15 and SEQ ID NOs: 127-158.

In some embodiments, the disclosure provides an isolated antibody or a fragment thereof that specifically binds to a human fibroblast growth factor 23 (FGF23) polypeptide, wherein the antibody or fragment thereof comprises a light chain complementarity determining region 3 (LCDR3) according to an amino acid sequence selected from SEQ ID NO: 16 and SEQ ID NOs: 159-185.

In some embodiments, the disclosure provides an isolated antibody or a fragment thereof that specifically binds to a human fibroblast growth factor 23 (FGF23) polypeptide, wherein the antibody or fragment thereof comprises a HCDR1, a HCDR2, a HCDR3, a LCDR1, a LCDR2, and a LCDR3, wherein: the HCDR1 comprises an amino acid sequence selected from SEQ ID NO: 11 and SEQ ID NOs: 22-32: the HCDR2 comprises an amino acid sequence selected from SEQ ID NO: 12 and SEQ ID NOs: 33-65: the HCDR3 comprises an amino acid sequence selected from SEQ ID NO: 13 and SEQ ID NOs: 66-93; the LCDR1 comprises an amino acid sequence selected from SEQ ID NO: 14 and SEQ ID NOs: 94-126; the LCDR2 comprises an amino acid sequence selected from SEQ ID NO: 15 and SEQ ID NOs: 127-158; and the LCDR3 comprises an amino acid sequence selected from SEQ ID NO: 16 and SEQ ID NOs: 159-185.

### Variable Regions of Certain Engineered FGF23-Binding Antibodies

Burosumab has a heavy chain variable region (VH) and a light chain variable region (VL) as follows: VH of amino acid sequence SEQ ID NO: 3, referred to herein as VH-001 and VL of amino acid sequence SEQ ID NO: 4, referred to herein as VL-001.

The FGF23-binding antibodies described herein may have one or more unique variable region domains relative to burosumab. That is, the FGF23-binding antibodies may have one or more amino acid change relative to the VH and/or the VL sequence of burosumab.

For instance, the isolated antibody or fragment thereof may comprise a heavy chain variable region (VH) and a light chain variable region (VL), wherein the VH comprises an amino acid sequence having at least 80% sequence identity to an amino acid sequence selected from SEQ ID NO: 3 and SEQ ID NOs: 186-270, or wherein the VL comprises an amino acid sequence having at least 80% sequence identity to an amino acid sequence selected from SEQ ID NO: 4 and SEQ ID NOs: 271-368.

The isolated antibody or fragment thereof may comprise a VH comprising an amino acid sequence having at least 90% sequence identity to an amino acid sequence selected from SEQ ID NO: 3 and SEQ ID NOs: 186-270, or the antibody or fragment thereof may comprise a VL comprising an amino acid sequence having at least 90% sequence identity to an amino acid sequence selected from SEQ ID NO: 4 and SEQ ID NOs: 271-368.

The isolated antibody or fragment thereof may comprise a VH comprising an amino acid sequence having at least 95% sequence identity to an amino acid sequence selected from SEQ ID NO: 3 and SEQ ID NOs: 186-270, or the antibody or fragment thereof may comprise a VL comprising an amino acid sequence having at least 95% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence having at least 80% sequence identity to an amino acid sequence selected from SEQ ID NO: 3 SEQ ID NOs: 186-270, and the VL may comprise an amino acid sequence having at least 80% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence having at least 90% sequence identity to an amino acid sequence selected from SEQ ID NO: 3 and SEQ ID NOs: 186-270, and the VL may comprise an amino acid sequence having at least 90% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence having at least 95% sequence identity to an amino acid sequence selected from SEQ ID NO: 3 and SEQ ID NOs: 186-270, and the VL may comprise an amino acid sequence having at least 95% sequence identity to an amino acid sequence selected from SEQ ID NO: 4 and SEQ ID NOs: 271-368.

The isolated antibody or fragment thereof may comprise a VH and a VL, wherein the VH may comprise an amino acid sequence selected from SEQ ID NO: 3 and SEQ ID NOs: 186-270, or the VL may comprise an amino acid sequence selected from SEQ ID NO: 4 and SEQ ID NOs: 271-368.

The isolated antibody or fragment thereof may comprise a VH and a VL, wherein the VH may comprise an amino acid sequence selected from SEQ ID NO: 3 and SEQ ID NOs: 186-270, and the VL may comprise an amino acid sequence selected from SEQ ID NO: 4 and SEQ ID NOs: 271-368.

Certain Engineered FGF23-Binding Antibodies with Double Amino Acid Changes

The FGF23-binding antibodies described herein may have one or more amino acid changes relative to the native burosumab amino acid sequence. For instance, the FGF23-binding antibodies may have at least two amino acid changes relative to burosumab. In some embodiments, the FGF23 binding antibodies have: one amino acid change to the heavy chain variable region relative to burosumab, and one amino acid change relative to the light chain variable region relative to burosumab; two amino acid changes relative to the heavy chain variable region relative to burosumab; or two amino acid changes relative to the light chain variable region relative to burosumab.

For instance, in some embodiments, the disclosure provides an isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 and a light chain variable region (VL) according to SEQ ID NO: 4, but comprising at least two amino acid changes in the VH and VL sequences, wherein the at least two amino acid changes are selected from VL:S31E/VL:D50F, VH:N31F/VH:S59F, VL:D50F/VL:S53E, VL:S31F/VL:S52E, VL:D50F/VH:V101I, VH:S59D/VH:V101I, VL:S31F/VL:S53E, VL:N92E/VL:D93F, VL:S31E/VL:S53E, VL:S31F/VL:D50F, VH:N31E/VH:S59E, VH:S57E/VH:S59D, VH:N31E/VH:I54F, VH:S59T/VH:V101L, VH:I54Y/VH:V101F, VH:N52F/VH:V101I, VL:N92D/VL:D93F, VH:N52F/VH:V101L, VL:S31F/VL:D50E, VL:Y94D/VH:S59D, VL:Y94D/VH:S59E, VL:N92F/VH:S59E, VL:N92Y/VH:S59E, VL:Y94E/VH:S59E, VL:S52D/VH:S59E, and VL:S52E/VH:S59E.

### Leader Sequences

The engineered FGF23-binding antibodies described herein may comprise a leader sequence at the N-terminus of the light chain and/or at the N-terminus of the heavy chain. In some embodiments, the leader sequence is a self-cleaving leader sequence. In some embodiments, the leader sequence facilitates expression, trafficking, stability, secretion, and/or purification of the heavy chain and/or light chain when expressed in a cell. In some embodiments, the leader sequence is a secretory signal to improve extracellular trafficking of the heavy chain and/or the light chain.

In certain embodiments, the engineered FGF23-binding antibodies described herein may comprise a leader sequence at the N-terminus of the heavy chain, wherein the leader sequence comprises the amino acid sequence MKWVTFISLLFLFSSAYS (SEQ ID NO: 369).

In certain embodiments, the engineered FGF23-binding antibodies described herein may comprise a leader sequence at the N-terminus of the light chain, wherein the leader sequence comprises the amino acid sequence MVSSAQFLGLLLLCFQGTRC (SEQ ID NO: 370).

### C-Terminal Lysine Clipping

The heavy chain of burosumab has a C-terminal lysine residue. It is known that antibody heavy chain C-terminal lysine residues are often clipped during bioproduction by carboxypeptidases present in cell culture or cell culture media. This phenomenon can give rise to variable amounts of process-related charge variants and non-homogeneous antibody preparations and thereby complicate efforts to manufacture homogeneous, safe, and effective lots of antibody (see, e.g., Faid et al., Eur J Pharm Sci., 2021 Apr. 1:159:105730). Accordingly, in some embodiments, the antibody variants described herein are modified to remove the heavy chain C-terminal lysine residue. That is, any heavy chain antibody sequence provided herein, including the parental burosumab heavy chain sequence (SEQ ID NO: 1) may be further modified to remove the C-terminal lysine residue (SEQ ID NO: 371). Thus, all variant heavy chain variable region (VH) sequences described in the present application are contemplated to be incorporated into full length heavy chain sequences with or without the C-terminal lysine residue.

### IV. Pharmaceutical Formulations

The human FGF23-binding antibodies described herein may be provided in pharmaceutical formulations. A pharmaceutical formulation can be capable of local or systemic administration. In certain aspects, the administration can be by any route, including intravenous, subcutaneous, pulmonary, intramuscular, intraperitoneal, dermal, oral, inhalation or nasal administration.

A pharmaceutical formulation of this disclosure may include carriers, diluents or excipients as are known in the art. Examples of pharmaceutical formulations and methods are described, for example, in Remington's Pharmaceutical Sciences, Mack Publishing Co. (A. R. Gennaro ed. 1985), and Remington, The Science and Practice of Pharmacy, 21st Edition (2005).

Examples of excipients for a pharmaceutical formulation include antioxidants, suspending agents, dispersing agents, preservatives, buffering agents, tonicity agents, and surfactants.

In certain embodiments, pharmaceutical formulations of this disclosure are solutions for subcutaneous injection comprising a human FGF23-binding antibody provided herein and one or more pharmaceutically acceptable excipient(s) selected from L-histidine. D-sorbitol E420, Polysorbate 80, L-methionine, Hydrochloric acid (for pH adjustment), and/or Water.

### V. Methods of Administration

Burosumab is indicated for subcutaneous use only. In pediatric XLH patients aged 6 months and older, starting dose regimen is 1 mg/kg of body weight rounded to the nearest 1 mg, administered every two weeks (Q2W) for patients weighing less than 10 kg. For XLH patients aged 6 months and older weighing 10 kg and greater, the starting dose regimen is 0.8 mg/kg of body weight rounded to the nearest 10 mg, administered every two weeks (Q2W). The minimum starting dose is 10 mg up to a maximum dose of 90 mg. The dose may be increased up to approximately 2 mg/kg (maximum 90 mg), administered every two weeks (Q2W) to achieve normal serum phosphorus. In adult XLH patients, the dose regimen is 1 mg/kg body weight rounded to the nearest 10 mg up to a maximum dose of 90 mg administered every four weeks (Q4W). In pediatric TIO patients aged 2 years and older, the starting dose is 0.4 mg/kg of body weight rounded to the nearest 10 mg every 2 weeks (Q2W). The dose may be increased up to 2 mg/kg not to exceed 180 mg, administered every two weeks (Q2W). In adult TIO patients, the starting dose is 0.5 mg/kg every four weeks (Q4W). The dose may be increased up to 2 mg/kg not to exceed 180 mg, administered every two weeks (Q2W).

Antibodies of the present disclosure may be administered according to the indicated uses of burosumab.

In some embodiments, the antibodies or pharmaceutical formulations thereof of the present disclosure can be administered at a therapeutically effective dose. A therapeutically effective dose can be an amount of an agent or formulation that is sufficient to cause a therapeutic effect in a subject administered the therapeutically effective dose.

Therapeutically effective doses of the human FGF23-binding antibodies provided herein, or pharmaceutical formulations thereof decrease serum FGF23 in subjects; increase renal maximum threshold for phosphate reabsorption (TmP) in subjects; increase serum inorganic phosphorus (Pi) in subjects; increase serum 1,25 dihydroxy vitamin D (1,25[OH]2D) concentration in subjects; and/or are useful for treating hypophosphatemic conditions in subjects including XLH or osteomalacia.

A therapeutically effective dose can be administered in one or more separate administrations, and by different routes. As will be appreciated in the art, a therapeutically effective dose or a therapeutically effective amount is largely determined based on the total amount of the therapeutic agent contained in the pharmaceutical formulation. Generally, a therapeutically effective amount is sufficient to achieve a meaningful benefit to the subject (e.g., treating, modulating, curing, preventing and/or ameliorating hypophosphatemia-related conditions). For example, a therapeutically effective amount may be an amount sufficient to achieve a desired therapeutic and/or prophylactic effect. Generally, the amount of a therapeutic agent (e.g., antibodies of the disclosure or functionally active fragments thereof) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

Methods provided herein contemplate single as well as multiple administrations of a therapeutically effective amount of the human FGF23-binding antibodies and pharmaceutical formulations thereof described herein. Pharmaceutical formulations comprising human FGF23-binding antibodies can be administered at regular intervals, depending on the nature, severity and extent of the subject's condition (e.g., the severity of a subject's disease state and the associated symptoms of hypophosphatemia). In some embodiments, a therapeutically effective amount of the human FGF23-binding antibodies and pharmaceutical formulations thereof of the present disclosure may be administered periodically at regular intervals (e.g., once every year, once every six months, once every four months, once every three months, once every two months, once a month), once every 4 weeks, once every 3 weeks, once every two weeks, weekly, daily, twice a day, three times a day, four times a day, five times a day, six times a day, or continuously. For example, a therapeutically effective amount of the human FGF23-binding antibodies and pharmaceutical formulations thereof of the present disclosure may be administered weekly, once every two weeks, or monthly.

Administering a therapeutically effective amount of the human FGF23-binding antibodies or formulations thereof provided herein may comprise administering to the subject between about 0.1 milligram of antibody per kilogram (mg/kg) body weight of the subject and about 100 mg/kg body weight of the subject, between about 0.1 mg/kg and about 50 mg/kg, between about 0.1 mg/kg and about 20 mg/kg, between about 0.1 mg/kg and about 10 mg/kg, between about 0.1 mg/kg and about 1 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5 mg/kg. In some embodiments, administering a therapeutically effective amount of the human FGF23-binding antibodies or formulations thereof provided herein may comprise administering less than 4 mg/kg, less than 3 mg/kg, less than 2 mg/k, or less than 1 mg/kg to the subject.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited steps.

In the application, where an element or component is said to be included in and/or selected from a list of recited elements or components, it will be understood that the element or component can be any one of the recited elements or components, or the element or component can be selected from a group consisting of two or more of the recited elements or components.

Further, it will be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein. All of the features disclosed in this specification may be combined in any combination. Each feature disclosed in this specification may be replaced by an alternative feature serving the same, equivalent, or similar purpose.

It will be understood that the expression “at least one of” includes individually each of the recited objects after the expression and the various combinations of two or more of the recited objects unless otherwise understood from the context and use. The expression “and/or” in connection with three or more recited objects will be understood to have the same meaning unless otherwise understood from the context.

The use of the term “include,” “includes,” “including,” “have,” “has,” “having,” “contain,” “contains,” or “containing,” including grammatical equivalents thereof, will be understood generally as open-ended and non-limiting, for example, not excluding additional unrecited elements or steps, unless otherwise specifically stated or understood from the context.

The use of any and all examples, or exemplary language herein, for example, “for instance”, “such as”, “for example”, “e.g.”, or “including” is intended merely to illustrate better the present disclosure and does not pose a limitation on the scope of the disclosure unless claimed. No language in the specification will be construed as indicating any non-claimed element as essential to the practice of the subject matter of the present disclosure.

It is understood that this disclosure is not limited to the particular methodology, protocols, materials, and reagents described, as these may vary. It is also to be understood that the terminology used herein is for the purpose of describing particular embodiments only, and is not intended to limit the scope of the present disclosure, which will be encompassed by the appended claims.

All publications, patents and patent applications, including any drawings, sequences, and appendices therein referred to throughout the present description are incorporated by reference in their entirety for all purposes to the same extent as if each individual publication, patent or patent application, drawing, sequence, or appendix was specifically and individually indicated to be incorporated by reference in its entirety for all purposes.

### EXAMPLES

The disclosure, now being generally described, will be more readily understood by reference to the following examples included for purposes of illustrating certain aspects and embodiments of the present disclosure without limiting the disclosure.

### Example 1. Antibody Structural Numbering (ASN #) System

Various numbering schemes are utilized for describing antibody amino acid sequences, including linear numbering, Kabat numbering, Chothia numbering, and Antibody Structural Numbering (ASN #) systems. The antibody residues described herein are presented according to the ASN #system and/or the linear numbering system. To facilitate conversion between these numbering schemes. Table 1 shows the burosumab parent light chain amino acid sequence with linear, Kabat, and ASN #systems and Table 2 shows the burosumab parent heavy chain amino acid sequence with linear, Kabat, and ASN #systems. In Table 1 and Table 2, the “Residue” columns show the amino acid residue from the burosumab light chain sequence of SEQ ID NO: 2 and the burosumab heavy chain sequence of SEQ ID NO: 1, respectively. In the “Kabat #” columns, the “L” designation refers to light chain and the H designation refers to heavy chain. In the “ASN #” columns, “KV” refers to light chain variable region, “KCnst-Ig” refers to light chain constant region, “HV” refers to heavy chain variable region. “HCnst-Ig” refers to heavy chain constant region. “Fc-N” refers to the CH2 region of the Fc, “Fc-C” refers to the CH3 region of the Fc, “HCnst-Po” refers to residues potentially attributable to the heavy chain constant region, and “Hinge” refers to hinge region residues.

TABLE 1
Burosumab light chain amino acid sequence numbering scheme conversion
table

| Residue | Linear # | Kabat # | ASN # | Residue | Linear # | Kabat # | ASN # |
|---|---|---|---|---|---|---|---|
| A | 1 | L1 | KV: 1 | F | 95 | L96 | KV: 137 |
| I | 2 | L2 | KV: 2 | T | 96 | L97 | KV: 138 |
| Q | 3 | L3 | KV: 3 | F | 97 | L98 | KV: 139 |
| L | 4 | L4 | KV: 4 | G | 98 | L99 | KV: 140 |
| T | 5 | L5 | KV: 5 | P | 99 | L100 | KV: 141 |
| Q | 6 | L6 | KV: 6 | G | 100 | L101 | KV: 142 |
| S | 7 | L7 | KV: 7 | T | 101 | L102 | KV: 143 |
| P | 8 | L8 | KV: 8 | K | 102 | L103 | KV: 144 |
| S | 9 | L9 | KV: 9 | V | 103 | L104 | KV: 145 |
| S | 10 | L10 | KV: 10 | D | 104 | L105 | KV: 146 |
| L | 11 | L11 | KV: 11 | I | 105 | L106 | KV: 147 |
| S | 12 | L12 | KV: 12 | K | 106 | L107 | KV: 148 |
| A | 13 | L13 | KV: 13 | R | 107 | L108 | KV: 149 |
| S | 14 | L14 | KV: 14 | — | 107.1 | | KCnst-Ig: 1 |
| V | 15 | L15 | KV: 15 | T | 108 | L109 | KCnst-Ig: 2 |
| G | 16 | L16 | KV: 16 | V | 109 | L110 | KCnst-Ig: 3 |
| D | 17 | L17 | KV: 17 | A | 110 | L111 | KCnst-Ig: 4 |
| R | 18 | L18 | KV: 18 | A | 111 | L112 | KCnst-Ig: 5 |
| V | 19 | L19 | KV: 19 | P | 112 | L113 | KCnst-Ig: 6 |
| T | 20 | L20 | KV: 20 | S | 113 | L114 | KCnst-Ig: 7 |
| I | 21 | L21 | KV: 21 | V | 114 | L115 | KCnst-Ig: 8 |
| T | 22 | L22 | KV: 22 | F | 115 | L116 | KCnst-Ig: 9 |
| C | 23 | L23 | KV: 23 | I | 116 | L117 | KCnst-Ig: 10 |
| R | 24 | L24 | KV: 24 | F | 117 | L118 | KCnst-Ig: 11 |
| A | 25 | L25 | KV: 25 | P | 118 | L119 | KCnst-Ig: 12 |
| S | 26 | L26 | KV: 26 | P | 119 | L120 | KCnst-Ig: 13 |
| — | 26.1 | | KV: 27 | S | 120 | L121 | KCnst-Ig: 14 |
| — | 26.2 | | KV: 28 | D | 121 | L122 | KCnst-Ig: 15 |
| Q | 27 | L27 | KV: 29 | E | 122 | L123 | KCnst-Ig: 16 |
| G | 28 | L28 | KV: 30 | — | 122.1 | | KCnst-Ig: 17 |
| I | 29 | L29 | KV: 31 | — | 122.2 | | KCnst-Ig: 18 |
| S | 30 | L30 | KV: 32 | Q | 123 | L124 | KCnst-Ig: 19 |
| S | 31 | L31 | KV: 33 | L | 124 | L125 | KCnst-Ig: 20 |
| — | 31.1 | | KV: 34 | — | 124.1 | | KCnst-Ig: 21 |
| — | 31.2 | | KV: 35 | — | 124.2 | | KCnst-Ig: 22 |
| — | 31.3 | | KV: 36 | K | 125 | L126 | KCnst-Ig: 23 |
| — | 31.4 | | KV: 37 | S | 126 | L127 | KCnst-Ig: 24 |
| — | 31.5 | | KV: 38 | G | 127 | L128 | KCnst-Ig: 25 |
| — | 31.6 | | KV: 39 | T | 128 | L129 | KCnst-Ig: 26 |
| A | 32 | L32 | KV: 40 | A | 129 | L130 | KCnst-Ig: 27 |
| L | 33 | L33 | KV: 41 | S | 130 | L131 | KCnst-Ig: 28 |
| V | 34 | L34 | KV: 42 | V | 131 | L132 | KCnst-Ig: 29 |
| W | 35 | L35 | KV: 43 | V | 132 | L133 | KCnst-Ig: 30 |
| Y | 36 | L36 | KV: 44 | C | 133 | L134 | KCnst-Ig: 31 |
| Q | 37 | L37 | KV: 45 | L | 134 | L135 | KCnst-Ig: 32 |
| Q | 38 | L38 | KV: 46 | L | 135 | L136 | KCnst-Ig: 33 |
| K | 39 | L39 | KV: 47 | N | 136 | L137 | KCnst-Ig: 34 |
| P | 40 | L40 | KV: 48 | N | 137 | L138 | KCnst-Ig: 35 |
| G | 41 | L41 | KV: 49 | F | 138 | L139 | KCnst-Ig: 36 |
| K | 42 | L42 | KV: 50 | Y | 139 | L140 | KCnst-Ig: 37 |
| A | 43 | L43 | KV: 51 | P | 140 | L141 | KCnst-Ig: 38 |
| P | 44 | L44 | KV: 52 | — | 140.1 | | KCnst-Ig: 39 |
| K | 45 | L45 | KV: 53 | — | 140.2 | | KCnst-Ig: 40 |
| L | 46 | L46 | KV: 54 | R | 141 | L142 | KCnst-Ig: 41 |
| L | 47 | L47 | KV: 55 | E | 142 | L143 | KCnst-Ig: 42 |
| I | 48 | L48 | KV: 56 | A | 143 | L144 | KCnst-Ig: 43 |
| Y | 49 | L49 | KV: 57 | K | 144 | L145 | KCnst-Ig: 44 |
| D | 50 | L50 | KV: 58 | V | 145 | L146 | KCnst-Ig: 45 |
| — | 50.1 | | KV: 59 | Q | 146 | L147 | KCnst-Ig: 46 |
| — | 50.2 | | KV: 60 | W | 147 | L148 | KCnst-Ig: 47 |
| — | 50.3 | | KV: 61 | — | 147.1 | | KCnst-Ig: 48 |
| — | 50.4 | | KV: 62 | K | 148 | L149 | KCnst-Ig: 49 |
| — | 50.5 | | KV: 63 | V | 149 | L150 | KCnst-Ig: 50 |
| — | 50.6 | | KV: 64 | D | 150 | L151 | KCnst-Ig: 51 |
| — | 50.7 | | KV: 65 | N | 151 | L152 | KCnst-Ig: 52 |
| — | 50.8 | | KV: 66 | A | 152 | L153 | KCnst-Ig: 53 |
| A | 51 | L51 | KV: 67 | L | 153 | L154 | KCnst-Ig: 54 |
| S | 52 | L52 | KV: 68 | Q | 154 | L155 | KCnst-Ig: 55 |
| S | 53 | L53 | KV: 69 | S | 155 | L156 | KCnst-Ig: 56 |
| L | 54 | L54 | KV: 70 | G | 156 | L157 | KCnst-Ig: 57 |
| E | 55 | L55 | KV: 71 | N | 157 | L158 | KCnst-Ig: 58 |
| S | 56 | L56 | KV: 72 | S | 158 | L159 | KCnst-Ig: 59 |
| G | 57 | L57 | KV: 73 | Q | 159 | L160 | KCnst-Ig: 60 |
| V | 58 | L58 | KV: 74 | E | 160 | L161 | KCnst-Ig: 61 |
| P | 59 | L59 | KV: 75 | S | 161 | L162 | KCnst-Ig: 62 |
| S | 60 | L60 | KV: 76 | V | 162 | L163 | KCnst-Ig: 63 |
| R | 61 | L61 | KV: 77 | T | 163 | L164 | KCnst-Ig: 64 |
| F | 62 | L62 | KV: 78 | E | 164 | L165 | KCnst-Ig: 65 |
| S | 63 | L63 | KV: 79 | Q | 165 | L166 | KCnst-Ig: 66 |
| G | 64 | L64 | KV: 80 | D | 166 | L167 | KCnst-Ig: 67 |
| S | 65 | L65 | KV: 81 | — | 166.1 | | KCnst-Ig: 68 |
| G | 66 | L66 | KV: 82 | — | 166.2 | | KCnst-Ig: 69 |
| S | 67 | L67 | KV: 83 | — | 166.3 | | KCnst-Ig: 70 |
| G | 68 | L68 | KV: 84 | — | 166.4 | | KCnst-Ig: 71 |
| — | 68.1 | | KV: 85 | S | 167 | L168 | KCnst-Ig: 72 |
| — | 68.2 | | KV: 86 | K | 168 | L169 | KCnst-Ig: 73 |
| T | 69 | L69 | KV: 87 | D | 169 | L170 | KCnst-Ig: 74 |
| D | 70 | L70 | KV: 88 | S | 170 | L171 | KCnst-Ig: 75 |
| F | 71 | L71 | KV: 89 | T | 171 | L172 | KCnst-Ig: 76 |
| T | 72 | L72 | KV: 90 | Y | 172 | L173 | KCnst-Ig: 77 |
| L | 73 | L73 | KV: 91 | S | 173 | L174 | KCnst-Ig: 78 |
| T | 74 | L74 | KV: 92 | L | 174 | L175 | KCnst-Ig: 79 |
| I | 75 | L75 | KV: 93 | S | 175 | L176 | KCnst-Ig: 80 |
| S | 76 | L76 | KV: 94 | S | 176 | L177 | KCnst-Ig: 81 |
| S | 77 | L77 | KV: 95 | T | 177 | L178 | KCnst-Ig: 82 |
| L | 78 | L78 | KV: 96 | L | 178 | L179 | KCnst-Ig: 83 |
| Q | 79 | L79 | KV: 97 | T | 179 | L180 | KCnst-Ig: 84 |
| P | 80 | L80 | KV: 98 | L | 180 | L181 | KCnst-Ig: 85 |
| E | 81 | L81 | KV: 99 | S | 181 | L182 | KCnst-Ig: 86 |
| D | 82 | L82 | KV: 100 | K | 182 | L183 | KCnst-Ig: 87 |
| F | 83 | L83 | KV: 101 | A | 183 | L184 | KCnst-Ig: 88 |
| A | 84 | L84 | KV: 102 | D | 184 | L185 | KCnst-Ig: 89 |
| T | 85 | L85 | KV: 103 | Y | 185 | L186 | KCnst-Ig: 90 |
| Y | 86 | L86 | KV: 104 | — | 185.1 | | KCnst-Ig: 91 |
| Y | 87 | L87 | KV: 105 | E | 186 | L187 | KCnst-Ig: 92 |
| C | 88 | L88 | KV: 106 | K | 187 | L188 | KCnst-Ig: 93 |
| Q | 89 | L89 | KV: 107 | H | 188 | L189 | KCnst-Ig: 94 |
| Q | 90 | L90 | KV: 108 | K | 189 | L190 | KCnst-Ig: 95 |
| F | 91 | L91 | KV: 109 | V | 190 | L191 | KCnst-Ig: 96 |
| N | 92 | L92 | KV: 110 | — | 190.1 | | KCnst-Ig: 97 |
| — | 92.1 | | KV: 111 | — | 190.2 | | KCnst-Ig: 98 |
| — | 92.2 | | KV: 112 | Y | 191 | L192 | KCnst-Ig: 99 |
| — | 92.3 | | KV: 113 | A | 192 | L193 | KCnst-Ig: 100 |
| — | 92.4 | | KV: 114 | C | 193 | L194 | KCnst-Ig: 101 |
| — | 92.5 | | KV: 115 | E | 194 | L195 | KCnst-Ig: 102 |
| — | 92.6 | | KV: 116 | V | 195 | L196 | KCnst-Ig: 103 |
| — | 92.7 | | KV: 117 | T | 196 | L197 | KCnst-Ig: 104 |
| — | 92.8 | | KV: 118 | H | 197 | L198 | KCnst-Ig: 105 |
| — | 92.9 | | KV: 119 | Q | 198 | L199 | KCnst-Ig: 106 |
| — | 92.10 | | KV: 120 | G | 199 | L200 | KCnst-Ig: 107 |
| — | 92.11 | | KV: 121 | L | 200 | L201 | KCnst-Ig: 108 |
| — | 92.12 | | KV: 122 | S | 201 | L202 | KCnst-Ig: 109 |
| — | 92.13 | | KV: 123 | — | 201.1 | | KCnst-Ig: 110 |
| — | 92.14 | | KV: 124 | S | 202 | L203 | KCnst-Ig: 111 |
| — | 92.15 | | KV: 125 | P | 203 | L204 | KCnst-Ig: 112 |
| — | 92.16 | | KV: 126 | V | 204 | L205 | KCnst-Ig: 113 |
| — | 92.17 | | KV: 127 | T | 205 | L206 | KCnst-Ig: 114 |
| — | 92.18 | | KV: 128 | K | 206 | L207 | KCnst-Ig: 115 |
| — | 92.19 | | KV: 129 | S | 207 | L208 | KCnst-Ig: 116 |
| — | 92.20 | | KV: 130 | F | 208 | L209 | KCnst-Ig: 117 |
| — | 92.21 | | KV: 131 | N | 209 | L210 | KCnst-Ig: 118 |
| — | 92.22 | | KV: 132 | R | 210 | L211 | KCnst-Ig: 119 |
| — | 92.23 | | KV: 133 | G | 211 | L212 | KCnst-Ig: 120 |
| — | 92.24 | | KV: 134 | E | 212 | L213 | KCnst-Ig: 121 |
| D | 93 | L93 | KV: 135 | C | 213 | L214 | KCnst-Ig: 122 |
| Y | 94 | L94 | KV: 136 | — | 213.1 | | KCnst-Ig: 123 |

TABLE 2
Burosumab heavy chain amino acid sequence numbering scheme conversion
table

| | | | | | Linear | Kabat | |
|---|---|---|---|---|---|---|---|
| Residue | Linear # | Kabat # | ASN # | Residue | # | # | ASN # |
| Q | 1 | H1 | HV: 1 | T | 225 | H238 | Hinge: 112 |
| V | 2 | H2 | HV: 2 | C | 226 | H239 | Hinge: 113 |
| Q | 3 | H3 | HV: 3 | P | 227 | H240 | Hinge: 114 |
| L | 4 | H4 | HV: 4 | P | 228 | H241 | Hinge: 115 |
| V | 5 | H5 | HV: 5 | C | 229 | H242 | Hinge: 116 |
| Q | 6 | H6 | HV: 6 | P | 230 | H243 | Hinge: 117 |
| S | 7 | H7 | HV: 7 | A | 231 | H244 | Hinge: 118 |
| — | 7.1 | | HV: 8 | P | 232 | H245 | Hinge: 119 |
| G | 8 | H8 | HV: 9 | E | 233 | H246 | Hinge: 120 |
| A | 9 | H9 | HV: 10 | L | 234 | H247 | Hinge: 121 |
| E | 10 | H10 | HV: 11 | L | 235 | H248 | Hinge: 122 |
| V | 11 | H11 | HV: 12 | G | 236 | H249 | Hinge: 123 |
| K | 12 | H12 | HV: 13 | — | 236.1 | | Fc-N: 1 |
| K | 13 | H13 | HV: 14 | — | 236.2 | | Fc-N: 2 |
| P | 14 | H14 | HV: 15 | — | 236.3 | | Fc-N: 3 |
| G | 15 | H15 | HV: 16 | — | 236.4 | | Fc-N: 4 |
| A | 16 | H16 | HV: 17 | G | 237 | H250 | Fc-N: 5 |
| S | 17 | H17 | HV: 18 | P | 238 | H251 | Fc-N: 6 |
| V | 18 | H18 | HV: 19 | S | 239 | H252 | Fc-N: 7 |
| K | 19 | H19 | HV: 20 | V | 240 | H253 | Fc-N: 8 |
| V | 20 | H20 | HV: 21 | F | 241 | H254 | Fc-N: 9 |
| S | 21 | H21 | HV: 22 | L | 242 | H255 | Fc-N: 10 |
| C | 22 | H22 | HV: 23 | F | 243 | H256 | Fc-N: 11 |
| K | 23 | H23 | HV: 24 | P | 244 | H257 | Fc-N: 12 |
| A | 24 | H24 | HV: 25 | P | 245 | H258 | Fc-N: 13 |
| S | 25 | H25 | HV: 26 | — | 245.1 | | Fc-N: 14 |
| G | 26 | H26 | HV: 27 | K | 246 | H259 | Fc-N: 15 |
| — | 26.1 | | HV: 28 | P | 247 | H260 | Fc-N: 16 |
| Y | 27 | H27 | HV: 29 | K | 248 | H261 | Fc-N: 17 |
| T | 28 | H28 | HV: 30 | — | 248.1 | | Fc-N: 18 |
| F | 29 | H29 | HV: 31 | D | 249 | H262 | Fc-N: 19 |
| T | 30 | H30 | HV: 32 | T | 250 | H263 | Fc-N: 20 |
| N | 31 | H31 | HV: 33 | L | 251 | H264 | Fc-N: 21 |
| — | 31.1 | | HV: 34 | M | 252 | H265 | Fc-N: 22 |
| — | 31.2 | | HV: 35 | I | 253 | H266 | Fc-N: 23 |
| — | 31.3 | | HV: 36 | S | 254 | H267 | Fc-N: 24 |
| — | 31.4 | | HV: 37 | R | 255 | H268 | Fc-N: 25 |
| — | 31.5 | | HV: 38 | T | 256 | H269 | Fc-N: 26 |
| H | 32 | H32 | HV: 39 | P | 257 | H270 | Fc-N: 27 |
| Y | 33 | H33 | HV: 40 | E | 258 | H271 | Fc-N: 28 |
| M | 34 | H34 | HV: 41 | V | 259 | H272 | Fc-N: 29 |
| H | 35 | H35 | HV: 42 | T | 260 | H273 | Fc-N: 30 |
| W | 36 | H36 | HV: 43 | C | 261 | H274 | Fc-N: 31 |
| V | 37 | H37 | HV: 44 | V | 262 | H275 | Fc-N: 32 |
| R | 38 | H38 | HV: 45 | V | 263 | H276 | Fc-N: 33 |
| Q | 39 | H39 | HV: 46 | V | 264 | H277 | Fc-N: 34 |
| A | 40 | H40 | HV: 47 | D | 265 | H278 | Fc-N: 35 |
| P | 41 | H41 | HV: 48 | V | 266 | H279 | Fc-N: 36 |
| G | 42 | H42 | HV: 49 | S | 267 | H280 | Fc-N: 37 |
| Q | 43 | H43 | HV: 50 | H | 268 | H281 | Fc-N: 38 |
| G | 44 | H44 | HV: 51 | E | 269 | H282 | Fc-N: 39 |
| L | 45 | H45 | HV: 52 | D | 270 | H283 | Fc-N: 40 |
| E | 46 | H46 | HV: 53 | P | 271 | H284 | Fc-N: 41 |
| W | 47 | H47 | HV: 54 | E | 272 | H285 | Fc-N: 42 |
| M | 48 | H48 | HV: 55 | V | 273 | H286 | Fc-N: 43 |
| G | 49 | H49 | HV: 56 | K | 274 | H287 | Fc-N: 44 |
| I | 50 | H50 | HV: 57 | F | 275 | H288 | Fc-N: 45 |
| I | 51 | H51 | HV: 58 | N | 276 | H289 | Fc-N: 46 |
| N | 52 | H52 | HV: 59 | W | 277 | H290 | Fc-N: 47 |
| P | 53 | H52A | HV: 60 | — | 277.1 | | Fc-N: 48 |
| — | 53.1 | | HV: 61 | Y | 278 | H291 | Fc-N: 49 |
| — | 53.2 | | HV: 62 | V | 279 | H292 | Fc-N: 50 |
| — | 53.3 | | HV: 63 | D | 280 | H295 | Fc-N: 51 |
| I | 54 | H53 | HV: 64 | G | 281 | H296 | Fc-N: 52 |
| S | 55 | H54 | HV: 65 | V | 282 | H299 | Fc-N: 53 |
| G | 56 | H55 | HV: 66 | E | 283 | H300 | Fc-N: 54 |
| S | 57 | H56 | HV: 67 | — | 283.1 | | Fc-N: 55 |
| T | 58 | H57 | HV: 68 | — | 283.2 | | Fc-N: 56 |
| S | 59 | H58 | HV: 69 | V | 284 | H301 | Fc-N: 57 |
| N | 60 | H59 | HV: 70 | H | 285 | H302 | Fc-N: 58 |
| A | 61 | H60 | HV: 71 | N | 286 | H303 | Fc-N: 59 |
| Q | 62 | H61 | HV: 72 | A | 287 | H304 | Fc-N: 60 |
| K | 63 | H62 | HV: 73 | K | 288 | H305 | Fc-N: 61 |
| F | 64 | H63 | HV: 74 | T | 289 | H306 | Fc-N: 62 |
| Q | 65 | H64 | HV: 75 | K | 290 | H307 | Fc-N: 63 |
| G | 66 | H65 | HV: 76 | P | 291 | H308 | Fc-N: 64 |
| R | 67 | H66 | HV: 77 | R | 292 | H309 | Fc-N: 65 |
| V | 68 | H67 | HV: 78 | E | 293 | H310 | Fc-N: 66 |
| T | 69 | H68 | HV: 79 | E | 294 | H311 | Fc-N: 67 |
| M | 70 | H69 | HV: 80 | Q | 295 | H312 | Fc-N: 68 |
| T | 71 | H70 | HV: 81 | — | 295.1 | | Fc-N: 69 |
| R | 72 | H71 | HV: 82 | — | 295.2 | | Fc-N: 70 |
| D | 73 | H72 | HV: 83 | — | 295.3 | | Fc-N: 71 |
| T | 74 | H73 | HV: 84 | — | 295.4 | | Fc-N: 72 |
| S | 75 | H74 | HV: 85 | Y | 296 | H313 | Fc-N: 73 |
| T | 76 | H75 | HV: 86 | N | 297 | H314 | Fc-N: 74 |
| S | 77 | H76 | HV: 87 | S | 298 | H317 | Fc-N: 75 |
| T | 78 | H77 | HV: 88 | T | 299 | H318 | Fc-N: 76 |
| V | 79 | H78 | HV: 89 | Y | 300 | H319 | Fc-N: 77 |
| Y | 80 | H79 | HV: 90 | R | 301 | H320 | Fc-N: 78 |
| M | 81 | H80 | HV: 91 | V | 302 | H321 | Fc-N: 79 |
| E | 82 | H81 | HV: 92 | V | 303 | H322 | Fc-N: 80 |
| L | 83 | H82 | HV: 93 | S | 304 | H323 | Fc-N: 81 |
| S | 84 | H82A | HV: 94 | V | 305 | H324 | Fc-N: 82 |
| S | 85 | H82B | HV: 95 | L | 306 | H325 | Fc-N: 83 |
| L | 86 | H82C | HV: 96 | T | 307 | H326 | Fc-N: 84 |
| R | 87 | H83 | HV: 97 | V | 308 | H327 | Fc-N: 85 |
| S | 88 | H84 | HV: 98 | L | 309 | H328 | Fc-N: 86 |
| E | 89 | H85 | HV: 99 | H | 310 | H329 | Fc-N: 87 |
| D | 90 | H86 | HV: 100 | Q | 311 | H330 | Fc-N: 88 |
| T | 91 | H87 | HV: 101 | D | 312 | H331 | Fc-N: 89 |
| A | 92 | H88 | HV: 102 | W | 313 | H332 | Fc-N: 90 |
| V | 93 | H89 | HV: 103 | — | 313.1 | | Fc-N: 91 |
| Y | 94 | H90 | HV: 104 | L | 314 | H333 | Fc-N: 92 |
| Y | 95 | H91 | HV: 105 | N | 315 | H334 | Fc-N: 93 |
| C | 96 | H92 | HV: 106 | G | 316 | H335 | Fc-N: 94 |
| A | 97 | H93 | HV: 107 | K | 317 | H336 | Fc-N: 95 |
| R | 98 | H94 | HV: 108 | E | 318 | H337 | Fc-N: 96 |
| D | 99 | H95 | HV: 109 | — | 318.1 | | Fc-N: 97 |
| I | 100 | H96 | HV: 110 | — | 318.2 | | Fc-N: 98 |
| V | 101 | H97 | HV: 111 | Y | 319 | H338 | Fc-N: 99 |
| — | 101.1 | | HV: 112 | K | 320 | H339 | Fc-N: 100 |
| — | 101.2 | | HV: 113 | C | 321 | H340 | Fc-N: 101 |
| — | 101.3 | | HV: 114 | K | 322 | H341 | Fc-N: 102 |
| — | 101.4 | | HV: 115 | V | 323 | H342 | Fc-N: 103 |
| — | 101.5 | | HV: 116 | S | 324 | H343 | Fc-N: 104 |
| — | 101.6 | | HV: 117 | N | 325 | H344 | Fc-N: 105 |
| — | 101.7 | | HV: 118 | K | 326 | H345 | Fc-N: 106 |
| — | 101.8 | | HV: 119 | A | 327 | H346 | Fc-N: 107 |
| — | 101.9 | | HV: 120 | L | 328 | H347 | Fc-N: 108 |
| — | 101.10 | | HV: 121 | P | 329 | H348 | Fc-N: 109 |
| — | 101.11 | | HV: 122 | — | 329.1 | | Fc-N: 110 |
| — | 101.12 | | HV: 123 | — | 329.2 | | Fc-N: 111 |
| — | 101.13 | | HV: 124 | A | 330 | H349 | Fc-N: 112 |
| — | 101.14 | | HV: 125 | P | 331 | H350 | Fc-N: 113 |
| — | 101.15 | | HV: 126 | I | 332 | H351 | Fc-N: 114 |
| — | 101.16 | | HV: 127 | E | 333 | H352 | Fc-N: 115 |
| — | 101.17 | | HV: 128 | K | 334 | H353 | Fc-N: 116 |
| — | 101.18 | | HV: 129 | T | 335 | H354 | Fc-N: 117 |
| — | 101.19 | | HV: 130 | I | 336 | H355 | Fc-N: 118 |
| — | 101.20 | | HV: 131 | S | 337 | H357 | Fc-N: 119 |
| — | 101.21 | | HV: 132 | K | 338 | H358 | Fc-N: 120 |
| — | 101.22 | | HV: 133 | A | 339 | H359 | Fc-N: 121 |
| D | 102 | H98 | HV: 134 | K | 340 | H360 | Fc-N: 122 |
| A | 103 | H99 | HV: 135 | G | 341 | H361 | Fc-N: 123 |
| F | 104 | H100 | HV: 136 | — | 341.1 | | Fc-C: 1 |
| D | 105 | H101 | HV: 137 | Q | 342 | H363 | Fc-C: 2 |
| F | 106 | H102 | HV: 138 | P | 343 | H364 | Fc-C: 3 |
| W | 107 | H103 | HV: 139 | R | 344 | H365 | Fc-C: 4 |
| G | 108 | H104 | HV: 140 | E | 345 | H366 | Fc-C: 5 |
| Q | 109 | H105 | HV: 141 | P | 346 | H367 | Fc-C: 6 |
| G | 110 | H106 | HV: 142 | Q | 347 | H368 | Fc-C: 7 |
| T | 111 | H107 | HV: 143 | V | 348 | H369 | Fc-C: 8 |
| M | 112 | H108 | HV: 144 | Y | 349 | H370 | Fc-C: 9 |
| V | 113 | H109 | HV: 145 | T | 350 | H371 | Fc-C: 10 |
| T | 114 | H110 | HV: 146 | L | 351 | H372 | Fc-C: 11 |
| V | 115 | H111 | HV: 147 | P | 352 | H373 | Fc-C: 12 |
| S | 116 | H112 | HV: 148 | P | 353 | H374 | Fc-C: 13 |
| S | 117 | H113 | HV: 149 | — | 353.1 | | Fc-C: 14 |
| — | 117.1 | | HCnst-Ig: 1 | S | 354 | H375 | Fc-C: 15 |
| — | 117.2 | | HCnst-Ig: 2 | R | 355 | H376 | Fc-C: 16 |
| A | 118 | H114 | HCnst-Ig: 3 | D | 356 | H377 | Fc-C: 17 |
| S | 119 | H115 | HCnst-Ig: 4 | — | 356.1 | | Fc-C: 18 |
| T | 120 | H116 | HCnst-Ig: 5 | E | 357 | H378 | Fc-C: 19 |
| K | 121 | H117 | HCnst-Ig: 6 | L | 358 | H381 | Fc-C: 20 |
| G | 122 | H118 | HCnst-Ig: 7 | — | 358.1 | | Fc-C: 21 |
| P | 123 | H119 | HCnst-Ig: 8 | — | 358.2 | | Fc-C: 22 |
| S | 124 | H120 | HCnst-Ig: 9 | T | 359 | H382 | Fc-C: 23 |
| V | 125 | H121 | HCnst-Ig: 10 | K | 360 | H383 | Fc-C: 24 |
| F | 126 | H122 | HCnst-Ig: 11 | N | 361 | H384 | Fc-C: 25 |
| P | 127 | H123 | HCnst-Ig: 12 | Q | 362 | H385 | Fc-C: 26 |
| L | 128 | H124 | HCnst-Ig: 13 | V | 363 | H386 | Fc-C: 27 |
| A | 129 | H125 | HCnst-Ig: 14 | S | 364 | H387 | Fc-C: 28 |
| P | 130 | H126 | HCnst-Ig: 15 | L | 365 | H388 | Fc-C: 29 |
| — | 130.1 | | HCnst-Ig: 16 | T | 366 | H389 | Fc-C: 30 |
| S | 131 | H127 | HCnst-Ig: 17 | C | 367 | H390 | Fc-C: 31 |
| — | 131.1 | | HCnst-Ig: 18 | L | 368 | H391 | Fc-C: 32 |
| S | 132 | H128 | HCnst-Ig: 19 | V | 369 | H392 | Fc-C: 33 |
| K | 133 | H129 | HCnst-Ig: 20 | K | 370 | H393 | Fc-C: 34 |
| S | 134 | H130 | HCnst-Ig: 21 | G | 371 | H394 | Fc-C: 35 |
| T | 135 | H133 | HCnst-Ig: 22 | F | 372 | H395 | Fc-C: 36 |
| S | 136 | H134 | HCnst-Ig: 23 | Y | 373 | H396 | Fc-C: 37 |
| G | 137 | H135 | HCnst-Ig: 24 | P | 374 | H397 | Fc-C: 38 |
| G | 138 | H136 | HCnst-Ig: 25 | — | 374.1 | | Fc-C: 39 |
| T | 139 | H137 | HCnst-Ig: 26 | — | 374.2 | | Fc-C: 40 |
| A | 140 | H138 | HCnst-Ig: 27 | S | 375 | H398 | Fc-C: 41 |
| A | 141 | H139 | HCnst-Ig: 28 | D | 376 | H399 | Fc-C: 42 |
| L | 142 | H140 | HCnst-Ig: 29 | I | 377 | H400 | Fc-C: 43 |
| G | 143 | H141 | HCnst-Ig: 30 | A | 378 | H401 | Fc-C: 44 |
| C | 144 | H142 | HCnst-Ig: 31 | V | 379 | H402 | Fc-C: 45 |
| L | 145 | H143 | HCnst-Ig: 32 | E | 380 | H405 | Fc-C: 46 |
| V | 146 | H144 | HCnst-Ig: 33 | W | 381 | H406 | Fc-C: 47 |
| K | 147 | H145 | HCnst-Ig: 34 | — | 381.1 | | Fc-C: 48 |
| D | 148 | H146 | HCnst-Ig: 35 | E | 382 | H407 | Fc-C: 49 |
| Y | 149 | H147 | HCnst-Ig: 36 | S | 383 | H408 | Fc-C: 50 |
| F | 150 | H148 | HCnst-Ig: 37 | N | 384 | H410 | Fc-C: 51 |
| P | 151 | H149 | HCnst-Ig: 38 | G | 385 | H411 | Fc-C: 52 |
| — | 151.1 | | HCnst-Ig: 39 | Q | 386 | H414 | Fc-C: 53 |
| — | 151.2 | | HCnst-Ig: 40 | P | 387 | H415 | Fc-C: 54 |
| E | 152 | H150 | HCnst-Ig: 41 | — | 387.1 | | Fc-C: 55 |
| P | 153 | H151 | HCnst-Ig: 42 | — | 387.2 | | Fc-C: 56 |
| V | 154 | H152 | HCnst-Ig: 43 | E | 388 | H416 | Fc-C: 57 |
| T | 155 | H153 | HCnst-Ig: 44 | N | 389 | H417 | Fc-C: 58 |
| V | 156 | H154 | HCnst-Ig: 45 | N | 390 | H418 | Fc-C: 59 |
| S | 157 | H156 | HCnst-Ig: 46 | Y | 391 | H419 | Fc-C: 60 |
| W | 158 | H157 | HCnst-Ig: 47 | K | 392 | H420 | Fc-C: 61 |
| — | 158.1 | | HCnst-Ig: 48 | T | 393 | H421 | Fc-C: 62 |
| N | 159 | H162 | HCnst-Ig: 49 | T | 394 | H422 | Fc-C: 63 |
| S | 160 | H163 | HCnst-Ig: 50 | P | 395 | H423 | Fc-C: 64 |
| G | 161 | H164 | HCnst-Ig: 51 | P | 396 | H424 | Fc-C: 65 |
| A | 162 | H165 | HCnst-Ig: 52 | V | 397 | H425 | Fc-C: 66 |
| L | 163 | H166 | HCnst-Ig: 53 | L | 398 | H426 | Fc-C: 67 |
| T | 164 | H167 | HCnst-Ig: 54 | D | 399 | H427 | Fc-C: 68 |
| S | 165 | H168 | HCnst-Ig: 55 | — | 399.1 | | Fc-C: 69 |
| G | 166 | H169 | HCnst-Ig: 56 | — | 399.2 | | Fc-C: 70 |
| V | 167 | H171 | HCnst-Ig: 57 | — | 399.3 | | Fc-C: 71 |
| H | 168 | H172 | HCnst-Ig: 58 | — | 399.4 | | Fc-C: 72 |
| T | 169 | H173 | HCnst-Ig: 59 | S | 400 | H428 | Fc-C: 73 |
| — | 169.1 | | HCnst-Ig: 60 | D | 401 | H430 | Fc-C: 74 |
| — | 169.2 | | HCnst-Ig: 61 | G | 402 | H433 | Fc-C: 75 |
| — | 169.3 | H174 | HCnst-Ig: 62 | S | 403 | H434 | Fc-C: 76 |
| F | 170 | H175 | HCnst-Ig: 63 | F | 404 | H435 | Fc-C: 77 |
| P | 171 | H176 | HCnst-Ig: 64 | F | 405 | H436 | Fc-C: 78 |
| A | 172 | H177 | HCnst-Ig: 65 | L | 406 | H437 | Fc-C: 79 |
| V | 173 | H178 | HCnst-Ig: 66 | Y | 407 | H438 | Fc-C: 80 |
| L | 174 | H179 | HCnst-Ig: 67 | S | 408 | H439 | Fc-C: 81 |
| Q | 175 | | HCnst-Ig: 68 | K | 409 | H440 | Fc-C: 82 |
| — | 175.1 | | HCnst-Ig: 69 | L | 410 | H441 | Fc-C: 83 |
| — | 175.2 | | HCnst-Ig: 70 | T | 411 | H442 | Fc-C: 84 |
| — | 175.3 | | HCnst-Ig: 71 | V | 412 | H443 | Fc-C: 85 |
| — | 175.4 | | HCnst-Ig: 72 | D | 413 | H444 | Fc-C: 86 |
| S | 176 | H180 | HCnst-Ig: 73 | K | 414 | H445 | Fc-C: 87 |
| S | 177 | H182 | HCnst-Ig: 74 | S | 415 | H446 | Fc-C: 88 |
| G | 178 | H183 | HCnst-Ig: 75 | R | 416 | H447 | Fc-C: 89 |
| L | 179 | H184 | HCnst-Ig: 76 | W | 417 | H448 | Fc-C: 90 |
| Y | 180 | H185 | HCnst-Ig: 77 | — | 417.1 | | Fc-C: 91 |
| S | 181 | H186 | HCnst-Ig: 78 | Q | 418 | H449 | Fc-C: 92 |
| L | 182 | H187 | HCnst-Ig: 79 | Q | 419 | H450 | Fc-C: 93 |
| S | 183 | H188 | HCnst-Ig: 80 | G | 420 | H451 | Fc-C: 94 |
| S | 184 | H189 | HCnst-Ig: 81 | N | 421 | H452 | Fc-C: 95 |
| V | 185 | H190 | HCnst-Ig: 82 | V | 422 | H453 | Fc-C: 96 |
| V | 186 | H191 | HCnst-Ig: 83 | — | 422.1 | | Fc-C: 97 |
| T | 187 | H192 | HCnst-Ig: 84 | — | 422.2 | | Fc-C: 98 |
| V | 188 | H193 | HCnst-Ig: 85 | F | 423 | H454 | Fc-C: 99 |
| P | 189 | H194 | HCnst-Ig: 86 | S | 424 | H455 | Fc-C: 100 |
| S | 190 | H195 | HCnst-Ig: 87 | C | 425 | H456 | Fc-C: 101 |
| S | 191 | H196 | HCnst-Ig: 88 | S | 426 | H457 | Fc-C: 102 |
| S | 192 | H197 | HCnst-Ig: 89 | V | 427 | H458 | Fc-C: 103 |
| L | 193 | H198 | HCnst-Ig: 90 | M | 428 | H459 | Fc-C: 104 |
| — | 193.1 | | HCnst-Ig: 91 | H | 429 | H460 | Fc-C: 105 |
| G | 194 | H199 | HCnst-Ig: 92 | E | 430 | H461 | Fc-C: 106 |
| T | 195 | H200 | HCnst-Ig: 93 | A | 431 | H462 | Fc-C: 107 |
| Q | 196 | H203 | HCnst-Ig: 94 | L | 432 | H463 | Fc-C: 108 |
| T | 197 | H205 | HCnst-Ig: 95 | H | 433 | H464 | Fc-C: 109 |
| | 197.1 | | HCnst-Ig: 96 | — | 433.1 | | Fc-C: 110 |
| — | 197.2 | | HCnst-Ig: 97 | — | 433.2 | | Fc-C: 111 |
| — | 197.3 | | HCnst-Ig: 98 | N | 434 | H465 | Fc-C: 112 |
| Y | 198 | H206 | HCnst-Ig: 99 | H | 435 | H466 | Fc-C: 113 |
| I | 199 | H207 | HCnst-Ig: 100 | Y | 436 | H467 | Fc-C: 114 |
| C | 200 | H208 | HCnst-Ig: 101 | T | 437 | H468 | Fc-C: 115 |
| N | 201 | H209 | HCnst-Ig: 102 | — | 437.1 | | Fc-C: 116 |
| V | 202 | H210 | HCnst-Ig: 103 | Q | 438 | H469 | Fc-C: 117 |
| N | 203 | H211 | HCnst-Ig: 104 | K | 439 | H470 | Fc-C: 118 |
| H | 204 | H212 | HCnst-Ig: 105 | S | 440 | H471 | Fc-C: 119 |
| K | 205 | H213 | HCnst-Ig: 106 | L | 441 | H472 | Fc-C: 120 |
| P | 206 | H214 | HCnst-Ig: 107 | S | 442 | H473 | Fc-C: 121 |
| S | 207 | H215 | HCnst-Ig: 108 | L | 443 | H474 | Fc-C: 122 |
| N | 208 | H216 | HCnst-Ig: 109 | S | 444 | H475 | Fc-C: 123 |
| — | 208.1 | | HCnst-Ig: 110 | P | 445 | H476 | HCnst- |
| | | | | | | | Po: 1 |
| — | 208.2 | | HCnst-Ig: 111 | G | 446 | H477 | HCnst- |
| | | | | | | | Po: 2 |
| T | 209 | H217 | HCnst-Ig: 112 | K | 447 | H478 | HCnst- |
| | | | | | | | Po: 3 |
| K | 210 | H218 | HCnst-Ig: 113 | — | 447.1 | | HCnst- |
| | | | | | | | Po: 4 |
| V | 211 | H219 | HCnst-Ig: 114 | — | 447.2 | | HCnst- |
| | | | | | | | Po: 5 |
| D | 212 | H220 | HCnst-Ig: 115 | — | 447.3 | | HCnst- |
| | | | | | | | Po: 6 |
| K | 213 | H221 | HCnst-Ig: 116 | — | 447.4 | | HCnst- |
| | | | | | | | Po: 7 |
| — | 213.1 | | HCnst-Ig: 117 | — | 447.5 | | HCnst- |
| | | | | | | | Po: 8 |
| K | 214 | H222 | HCnst-Ig: 118 | — | 447.6 | | HCnst- |
| | | | | | | | Po: 9 |
| V | 215 | H223 | HCnst-Ig: 119 | — | 447.7 | | HCnst- |
| | | | | | | | Po: 10 |
| — | 215.1 | | HCnst-Ig: 120 | — | 447.8 | | HCnst- |
| | | | | | | | Po: 11 |
| — | 215.2 | | HCnst-Ig: 121 | — | 447.9 | | HCnst- |
| | | | | | | | Po: 12 |
| — | 215.3 | | HCnst-Ig: 122 | — | 447.10 | | HCnst- |
| | | | | | | | Po: 13 |
| — | 215.4 | | HCnst-Ig: 123 | — | 447.11 | | HCnst- |
| | | | | | | | Po: 14 |
| — | 215.5 | | Hinge: 1 | — | 447.12 | | HCnst- |
| | | | | | | | Po: 15 |
| — | 215.6 | | Hinge: 2 | — | 447.13 | | HCnst- |
| | | | | | | | Po: 16 |
| — | 215.7 | | Hinge: 3 | — | 447.14 | | HCnst- |
| | | | | | | | Po: 17 |
| — | 215.8 | | Hinge: 4 | — | 447.15 | | HCnst- |
| | | | | | | | Po: 18 |
| — | 215.9 | | Hinge: 5 | — | 447.16 | | HCnst- |
| | | | | | | | Po: 19 |
| — | 215.10 | | Hinge: 6 | — | 447.17 | | HCnst- |
| | | | | | | | Po: 20 |
| — | 215.11 | | Hinge: 7 | — | 447.18 | | HCnst- |
| | | | | | | | Po: 21 |
| — | 215.12 | | Hinge: 8 | — | 447.19 | | HCnst- |
| | | | | | | | Po: 22 |
| — | 215.13 | | Hinge: 9 | — | 447.20 | | HCnst- |
| | | | | | | | Po: 23 |
| — | 215.14 | | Hinge: 10 | — | 447.21 | | HCnst- |
| | | | | | | | Po: 24 |
| — | 215.15 | | Hinge: 11 | — | 447.22 | | HCnst- |
| | | | | | | | Po: 25 |
| — | 215.16 | | Hinge: 12 | — | 447.23 | | HCnst- |
| | | | | | | | Po: 26 |
| — | 215.17 | | Hinge: 13 | — | 447.24 | | HCnst- |
| | | | | | | | Po: 27 |
| — | 215.18 | | Hinge: 14 | — | 447.25 | | HCnst- |
| | | | | | | | Po: 28 |
| — | 215.19 | | Hinge: 15 | — | 447.26 | | HCnst- |
| | | | | | | | Po: 29 |
| — | 215.20 | | Hinge: 16 | — | 447.27 | | HCnst- |
| | | | | | | | Po: 30 |
| — | 215.21 | | Hinge: 17 | — | 447.28 | | HCnst- |
| | | | | | | | Po: 31 |
| — | 215.22 | | Hinge: 18 | — | 447.29 | | HCnst- |
| | | | | | | | Po: 32 |
| — | 215.23 | | Hinge: 19 | — | 447.30 | | HCnst- |
| | | | | | | | Po: 33 |
| — | 215.24 | | Hinge: 20 | — | 447.31 | | HCnst- |
| | | | | | | | Po: 34 |
| — | 215.25 | | Hinge: 21 | — | 447.32 | | HCnst- |
| | | | | | | | Po: 35 |
| — | 215.26 | | Hinge: 22 | — | 447.33 | | HCnst- |
| | | | | | | | Po: 36 |
| — | 215.27 | | Hinge: 23 | — | 447.34 | | HCnst- |
| | | | | | | | Po: 37 |
| — | 215.28 | | Hinge: 24 | — | 447.35 | | HCnst- |
| | | | | | | | Po: 38 |
| — | 215.29 | | Hinge: 25 | — | 447.36 | | HCnst- |
| | | | | | | | Po: 39 |
| — | 215.30 | | Hinge: 26 | — | 447.37 | | HCnst- |
| | | | | | | | Po: 40 |
| — | 215.31 | | Hinge: 27 | — | 447.38 | | HCnst- |
| | | | | | | | Po: 41 |
| — | 215.32 | | Hinge: 28 | — | 447.39 | | HCnst- |
| | | | | | | | Po: 42 |
| — | 215.33 | | Hinge: 29 | — | 447.40 | | HCnst- |
| | | | | | | | Po: 43 |
| — | 215.34 | | Hinge: 30 | — | 447.41 | | HCnst- |
| | | | | | | | Po: 44 |
| — | 215.35 | | Hinge: 31 | — | 447.42 | | HCnst- |
| | | | | | | | Po: 45 |
| — | 215.36 | | Hinge: 32 | — | 447.43 | | HCnst- |
| | | | | | | | Po: 46 |
| — | 215.37 | | Hinge: 33 | — | 447.44 | | HCnst- |
| | | | | | | | Po: 47 |
| — | 215.38 | | Hinge: 34 | — | 447.45 | | HCnst- |
| | | | | | | | Po: 48 |
| — | 215.39 | | Hinge: 35 | — | 447.46 | | HCnst- |
| | | | | | | | Po: 49 |
| — | 215.40 | | Hinge: 36 | — | 447.47 | | HCnst- |
| | | | | | | | Po: 50 |
| — | 215.41 | | Hinge: 37 | — | 447.48 | | HCnst- |
| | | | | | | | Po: 51 |
| — | 215.42 | | Hinge: 38 | — | 447.49 | | HCnst- |
| | | | | | | | Po: 52 |
| — | 215.43 | | Hinge: 39 | — | 447.50 | | HCnst- |
| | | | | | | | Po: 53 |
| — | 215.44 | | Hinge: 40 | — | 447.51 | | HCnst- |
| | | | | | | | Po: 54 |
| — | 215.45 | | Hinge: 41 | — | 447.52 | | HCnst- |
| | | | | | | | Po: 55 |
| — | 215.46 | | Hinge: 42 | — | 447.53 | | HCnst- |
| | | | | | | | Po: 56 |
| — | 215.47 | | Hinge: 43 | — | 447.54 | | HCnst- |
| | | | | | | | Po: 57 |
| — | 215.48 | | Hinge: 44 | — | 447.55 | | HCnst- |
| | | | | | | | Po: 58 |
| — | 215.49 | | Hinge: 45 | — | 447.56 | | HCnst- |
| | | | | | | | Po: 59 |
| — | 215.50 | | Hinge: 46 | — | 447.57 | | HCnst- |
| | | | | | | | Po: 60 |
| — | 215.51 | | Hinge: 47 | — | 447.58 | | HCnst- |
| | | | | | | | Po: 61 |
| — | 215.52 | | Hinge: 48 | — | 447.59 | | HCnst- |
| | | | | | | | Po: 62 |
| — | 215.53 | | Hinge: 49 | — | 447.60 | | HCnst- |
| | | | | | | | Po: 63 |
| — | 215.54 | | Hinge: 50 | — | 447.61 | | HCnst- |
| | | | | | | | Po: 64 |
| — | 215.55 | | Hinge: 51 | — | 447.62 | | HCnst- |
| | | | | | | | Po: 65 |
| — | 215.56 | | Hinge: 52 | — | 447.63 | | HCnst- |
| | | | | | | | Po: 66 |
| — | 215.57 | | Hinge: 53 | — | 447.64 | | HCnst- |
| | | | | | | | Po: 67 |
| — | 215.58 | | Hinge: 54 | — | 447.65 | | HCnst- |
| | | | | | | | Po: 68 |
| — | 215.59 | | Hinge: 55 | — | 447.66 | | HCnst- |
| | | | | | | | Po: 69 |
| — | 215.60 | | Hinge: 56 | — | 447.67 | | HCnst- |
| | | | | | | | Po: 70 |
| — | 215.61 | | Hinge: 57 | — | 447.68 | | HCnst- |
| | | | | | | | Po: 71 |
| — | 215.62 | | Hinge: 58 | — | 447.69 | | HCnst- |
| | | | | | | | Po: 72 |
| — | 215.63 | | Hinge: 59 | — | 447.70 | | HCnst- |
| | | | | | | | Po: 73 |
| — | 215.64 | | Hinge: 60 | — | 447.71 | | HCnst- |
| | | | | | | | Po: 74 |
| — | 215.65 | | Hinge: 61 | — | 447.72 | | HCnst- |
| | | | | | | | Po: 75 |
| — | 215.66 | | Hinge: 62 | — | 447.73 | | HCnst- |
| | | | | | | | Po: 76 |
| — | 215.67 | | Hinge: 63 | — | 447.74 | | HCnst- |
| | | | | | | | Po: 77 |
| — | 215.68 | | Hinge: 64 | — | 447.75 | | HCnst- |
| | | | | | | | Po: 78 |
| — | 215.69 | | Hinge: 65 | — | 447.76 | | HCnst- |
| | | | | | | | Po: 79 |
| — | 215.70 | | Hinge: 66 | — | 447.77 | | HCnst- |
| | | | | | | | Po: 80 |
| — | 215.71 | | Hinge: 67 | — | 447.78 | | HCnst- |
| | | | | | | | Po: 81 |
| — | 215.72 | | Hinge: 68 | — | 447.79 | | HCnst- |
| | | | | | | | Po: 82 |
| — | 215.73 | | Hinge: 69 | — | 447.80 | | HCnst- |
| | | | | | | | Po: 83 |
| — | 215.74 | | Hinge: 70 | — | 447.81 | | HCnst- |
| | | | | | | | Po: 84 |
| — | 215.75 | | Hinge: 71 | — | 447.82 | | HCnst- |
| | | | | | | | Po: 85 |
| — | 215.76 | | Hinge: 72 | — | 447.83 | | HCnst- |
| | | | | | | | Po: 86 |
| — | 215.77 | | Hinge: 73 | — | 447.84 | | HCnst- |
| | | | | | | | Po: 87 |
| — | 215.78 | | Hinge: 74 | — | 447.85 | | HCnst- |
| | | | | | | | Po: 88 |
| — | 215.79 | | Hinge: 75 | — | 447.86 | | HCnst- |
| | | | | | | | Po: 89 |
| — | 215.80 | | Hinge: 76 | — | 447.87 | | HCnst- |
| | | | | | | | Po: 90 |
| — | 215.81 | | Hinge: 77 | — | 447.88 | | HCnst- |
| | | | | | | | Po: 91 |
| — | 215.82 | | Hinge: 78 | — | 447.89 | | HCnst- |
| | | | | | | | Po: 92 |
| — | 215.83 | | Hinge: 79 | — | 447.90 | | HCnst- |
| | | | | | | | Po: 93 |
| — | 215.84 | | Hinge: 80 | — | 447.91 | | HCnst- |
| | | | | | | | Po: 94 |
| — | 215.85 | | Hinge: 81 | — | 447.92 | | HCnst- |
| | | | | | | | Po: 95 |
| — | 215.86 | | Hinge: 82 | — | 447.93 | | HCnst- |
| | | | | | | | Po: 96 |
| — | 215.87 | | Hinge: 83 | — | 447.94 | | HCnst- |
| | | | | | | | Po: 97 |
| — | 215.88 | | Hinge: 84 | — | 447.95 | | HCnst- |
| | | | | | | | Po: 98 |
| — | 215.89 | | Hinge: 85 | — | 447.96 | | HCnst- |
| | | | | | | | Po: 99 |
| — | 215.90 | | Hinge: 86 | — | 447.97 | | HCnst- |
| | | | | | | | Po: 100 |
| — | 215.91 | | Hinge: 87 | — | 447.98 | | HCnst- |
| | | | | | | | Po: 101 |
| — | 215.92 | | Hinge: 88 | — | 447.99 | | HCnst- |
| | | | | | | | Po: 102 |
| — | 215.93 | | Hinge: 89 | — | 447.100 | | HCnst- |
| | | | | | | | Po: 103 |
| — | 215.94 | | Hinge: 90 | — | 447.101 | | HCnst- |
| | | | | | | | Po: 104 |
| — | 215.95 | | Hinge: 91 | — | 447.102 | | HCnst- |
| | | | | | | | Po: 105 |
| — | 215.96 | | Hinge: 92 | — | 447.103 | | HCnst- |
| | | | | | | | Po: 106 |
| — | 215.97 | | Hinge: 93 | — | 447.104 | | HCnst- |
| | | | | | | | Po: 107 |
| — | 215.98 | | Hinge: 94 | — | 447.105 | | HCnst- |
| | | | | | | | Po: 108 |
| — | 215.99 | | Hinge: 95 | — | 447.106 | | HCnst- |
| | | | | | | | Po: 109 |
| E | 216 | H226 | Hinge: 96 | — | 447.107 | | HCnst- |
| | | | | | | | Po: 110 |
| P | 217 | H227 | Hinge: 97 | — | 447.108 | | HCnst- |
| | | | | | | | Po: 111 |
| K | 218 | H228 | Hinge: 98 | — | 447.109 | | HCnst- |
| | | | | | | | Po: 112 |
| S | 219 | H232 | Hinge: 99 | — | 447.110 | | HCnst- |
| | | | | | | | Po: 113 |
| — | 219.1 | | Hinge: 100 | — | 447.111 | | HCnst- |
| | | | | | | | Po: 114 |
| — | 219.2 | | Hinge: 101 | — | 447.112 | | HCnst- |
| | | | | | | | Po: 115 |
| — | 219.3 | | Hinge: 102 | — | 447.113 | | HCnst- |
| | | | | | | | Po: 116 |
| — | 219.4 | | Hinge: 103 | — | 447.114 | | HCnst- |
| | | | | | | | Po: 117 |
| — | 219.5 | | Hinge: 104 | — | 447.115 | | HCnst- |
| | | | | | | | Po: 118 |
| — | 219.6 | | Hinge: 105 | — | 447.116 | | HCnst- |
| | | | | | | | Po: 119 |
| — | 219.7 | | Hinge: 106 | — | 447.117 | | HCnst- |
| | | | | | | | Po: 120 |
| C | 220 | H233 | Hinge: 107 | — | 447.118 | | HCnst- |
| | | | | | | | Po: 121 |
| D | 221 | H234 | Hinge: 108 | — | 447.119 | | HCnst- |
| | | | | | | | Po: 122 |
| K | 222 | H235 | Hinge: 109 | — | 447.120 | | HCnst- |
| | | | | | | | Po: 123 |
| T | 223 | H236 | Hinge: 110 | | | | |
| H | 224 | H237 | Hinge: 111 | | | | |

### Example 2: Epitope Mapping of Burosumab-Human FGF23

Hydrogen deuterium exchange mass spectrometry (HDX-MS) measures the rate of solvent exchange, i.e., the phenomenon whereby atoms comprising the solvent with a heavier hydrogen isotope are swapped for protein atoms within the backbone amide bonds. This approach makes it possible to determine solvent uptake by proteins by exposing the proteins to a deuterated solvent for a defined period, quenching the solvent exchange reaction, and then measuring the increase in mass of the proteins by mass spectrometry. Typically, a ligand binding event results in a localized reduction in the deuteration uptake at the ligand interaction site, enabling approximate mapping of regions involved in, e.g., antibody/target epitope and paratope interaction.

In attempt to map the burosumab epitopes on human FGF23, HDX-MS was performed using FGF23 (R179Q) produced in HEK293S cells. The burosumab was produced in CHO cells (while Engineered FGF23-binding antibodies in later examples were expressed in HEK293S cells). Antigen-antibody complexes were formed by combining burosumab and FGF23 (R179Q) in solution and then diluted 1:10 in ice-cold deuterated assay buffer for 60 seconds, 600 seconds, 1800 seconds, or 3600 seconds. Briefly, the HDX-MS assay utilized PBS at pH 7.4 as the assay buffer. Quench buffer with 1 M glycine. 4M guanidine HCl, and 400 mM TCEP at pH 2.5 was used. 100 pmol were injected in duplicate at 1:1 ratio of assay buffer-to-quench buffer. Antigen concentration during labeling was 1.5 μM and antibody concentration during labeling was 1 μM. The deuteration reaction was quenched using 1:1 addition of quench buffer (pH 2.5) and the solution subsequently loaded on a liquid chromatography (LC) column for on-column proteolytic digestion followed by LC separation and mass spectrometry.

The HDX-MS results are shown in FIG. 1. The y-axis shows % deuteration (% D), i.e., the peptide-length-normalized difference in deuteration between unbound FGF23 and antibody-bound FGF23. Values less than zero (0) reflect decreased deuteration. i.e., protection against the deuteration reaction by antibody-antigen binding. Values greater than zero (0) reflect increased deuteration, i.e., exposure to the deuteration reaction. The x-axis shows FGF23 amino acid residues 1-251. Colored horizontal bars represent deuterium-labeling time point for each identified peptide (black=60 s, blue=600 s, orange=1800 s, magenta=3600 s). The C-terminal region of FGF23 is not represented due to the lack of sequence coverage for the region comprising approximately amino acid residue 158 through 251. It is believed that endogenous glycosylation in this region of the protein prevents accurate measurement of deuterium exchange in the present assay. Repeat examples following enzymatic de-glycosylation of FGF23 support this hypothesis (data not shown). Overall, three regions of strong protection (red boxes) were identified where % deuteration was low and/or labeling time points were short, relatively (protected region 1, protected region 2, and protected region 3). Protected region 1 (Y43 to Y70) had the weakest protection of the three strong protection regions. Protected region 2 (A80 to R92) had stronger protection than protected region 1. Protected region 3 (L135 to F157) had the strongest protection of the three.

Based on the HDX-MS data, protected regions 1-3 on FGF23 were hypothesized to be involved in antibody binding or be protected due to conformational change in the presence of antibody.

These three protected regions were mapped to a crystal structure of FGF23 (R179Q) (the “2P39” structure; see Goetz et al. “Molecular insights into the klotho-dependent, endocrine mode of action of fibroblast growth factor 19 subfamily members” Mol Cell Biol (2007) 27 p. 3417-28) to visualize regions with likely antibody binding interaction. FIG. 2 shows the 23P9 crystal structure of FGF23 with protected regions defined as per findings from HDX-MS analysis: amino acids ~43 to ~70 (protected region 1) in purple, amino acids ~80 to ~92 (protected region 2) in green, and amino acids ~135 to ~157 (protected region 3) in red.

In sum, HDX-MS identified three FGF23 (R179Q) regions that are protected by in the presence of burosumab, shown in Table 3:

TABLE 3
FGF23 protected regions by HDX-MS

| | Amino acid | | |
|---|---|---|---|
| | position | | |
| | (relative to | | Amino acid |
| FGF23 | SEQ ID NO: | | sequence |
| (R179Q) | 17 or 18) | Amino acid sequence | SEQ ID NO |
| Protected | 43-70 | YTATARNSYHLQIHKNGHVDGAPHQTIY | 19 |
| region 1 | | | |
| Protected | 80-92 | AGFVVITGVMSRR | 20 |
| region 2 | | | |
| Protected | 135-157 | LVSLGRAKRAFLPGMNPPPYSQF | 21 |
| region 3 | | | |

The HDX-MS data suggest the strongest protected regions (2 and 3) represent a possible burosumab epitope in the proximity of residues 82 to 90 and 136 to 140 of FGF23 (R179Q). Within protected region 3, residues L135, V136, 5137, L138, G139, R140, and A141 represent the strongest area of protection and thus may be among the most important residues involved in the FGF23-burosumab interaction.

Comparing these findings to previous efforts at mapping the burosumab epitopes on human FGF23, it was observed that protected region 1 encompasses residues of FN1-Epitope 1 and FN1-Epitope 2 identified by Yamazaki et al. “Anti-FGF23 neutralizing antibodies show the physiological role and structural features of FGF23.” Journal of Bone and Mineral Research 23.9 (2008): 1509-1518. However. FN1-Epitope 3 and FN1-Epitope 4 fall outside the protected region 1, protected region 2, and protected region 3 regions identified in the present study, suggesting the FN1 antibody previously characterized by Yamazaki et al. recognizes a different face on FGF23, compared to burosumab. Further, Kanhasut et al. 2022 identified epitopes that largely correspond with those previously described by Yamazaki et al., further demonstrating the importance of the present comprehensive study in which the hFGF23:burosumab binding surface has been definitively defined by crystallographic analyses and supported by HDX-MS.

### Example 3: Paratope Mapping of Burosumab-Human FGF23

Paratope residues/regions of burosumab for binding human FGF23 were interrogated using HDX-MS in like manner as FGF23 epitope mapping described in Example 2. FGF23 (R179Q) was produced in HEK293S cells. The burosumab was produced in CHO cells (while Engineered FGF23-binding antibodies in later examples were expressed in HEK293S cells). Antigen-antibody complexes were formed by combining burosumab and FGF23 (R179Q) in solution and then diluted 1:10 in ice-cold deuterated assay buffer for 60 seconds, 600 seconds, 1800 seconds, or 3600 seconds. The deuteration reaction was quenched using 1:1 addition of quench buffer (pH 2.5) and the solution subsequently loaded on a liquid chromatography (LC) column for on-column proteolytic digestion followed by LC separation and mass spectrometry.

Briefly, the HDX-MS assay utilized PBS at pH 7.4 as the assay buffer. Quench buffer with 1 M glycine, 4M guanidine HCl, and 400 mM TCEP at pH 2.5 was used. 100 pmol were injected in duplicate at 1:1 ratio of assay buffer-to-quench buffer. Labeling (deuteration) was carried out for 15 second, 60 seconds. 600 seconds, and 3600 seconds. Antigen concentration during labeling was 1.5 μM and antibody concentration during labeling was 1 μM.

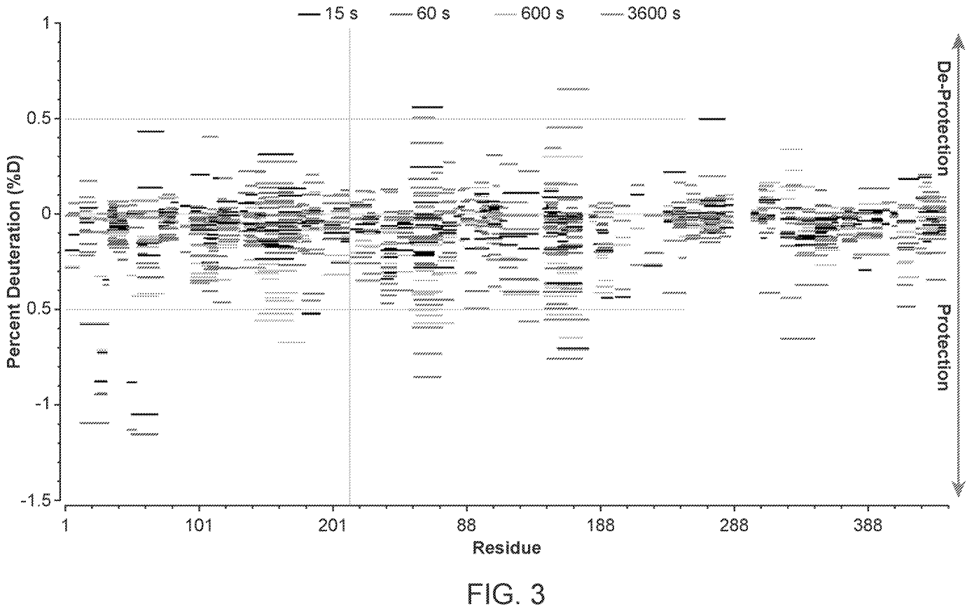

The HDX-MS results are shown in FIG. 3. The y-axis shows % deuteration (% D), i.e., the peptide-length-normalized difference in deuteration between unbound antibody and FGF23-bound antibody. Values less than 0 reflect decreased deuteration, i.e., protection against the deuteration reaction by antibody-antigen binding. Values greater than 0 reflect increased deuteration, i.e., exposure to the deuteration reaction. The x-axis shows burosumab amino acid residues (light chain on the left and heavy chain on the right separated by vertical bar). Colored horizontal bars represent deuterium-labeling time point for each identified peptide (black=15 s, blue=60 s, orange=600 s, magenta=3600 s).

Protection on the burosumab antibody was observed and considered to represent conformational protection of the protected regions from deuteration and/or antigen (FGF23) binding. In particular, the following regions on the burosumab light chain and heavy chain sequences shown in Table 4 were identified as showing reduced deuteration by HDX-MS.

TABLE 4
Burosumab paratopes by HDX-MS

| | Amino acid | | |
|---|---|---|---|
| | position | | Amino acid |
| | (relative to | | sequence SEQ |
| Burosumab | SEQ ID NO: 1) | Amino acid sequence | ID NO |
| Heavy chain | 24-35 | ASGYTFTNHYMH | 5 |
| paratope region 1 | | | |
| Heavy chain | 49-69 | GIINPISGSTSNAQKFQG | 6 |
| paratope region 2 | | RVT | |
| Heavy chain | 148-163 | DYFPEPVTVSWNSGAL | 7 |
| paratope region 3 | | | |
| | Amino acid | | |
| | position | | Amino acid |
| | (relative to | | sequence SEQ |
| Burosumab | SEQ ID NO: 2) | Amino acid sequence | ID NO |
| Light chain | 23-33 | CRASQGISSAL | 8 |
| paratope region 1 | | | |
| Light chain | 50-70 | DASSLESGVPSRFSGSGS | 9 |
| paratope region 2 | | GTD | |
| Light chain | 103-122 | VDIKRTVAAPSVFIFPPS | 10 |
| paratope region 3 | | DE | |

Among these, burosumab heavy chain paratope region 1 (SEQ ID NO: 5) and light chain paratope region 1 (SEQ ID NO: 8) were hypothesized to be involved in antigen binding.

### Example 4: Molecular Docking to Evaluate Burosumab-FGF23 Interaction

Protein-protein docking was used to predict the three dimensional structure of the burosumab—FGF23 complex and identify antibody-antigen interacting surfaces and residues implicated in binding. Even without a crystal structure of the Fab-antigen complex, computational docking was utilized to gain insights into the interaction of the burosumab Fab fragment and FGF23 structures.

A crystal structure of the FGF23 in complex with sucrose octasulfate is available (“2P39”; see Goetz et al. “Molecular insights into the klotho-dependent, endocrine mode of action of fibroblast growth factor 19 subfamily members” Mol Cell Biol (2007) 27 p. 3417-28, incorporated herein by reference). In addition, a crystal structure of the FGF23-FGFR1c-aKlotho temary complex is likewise available (“5W21”; see Chen et al. “alpha-Klotho is anon-enzymatic molecular scaffold for FGF23 hormone signaling” (2018) Nature 553: 461-466, incorporated herein by reference). These available crystal structures were utilized to perform molecular docking studies of human FGF23 with the known crystal structure of burosumab Fab (“7VEN”; Heo, Y. S. “Structure of burosumab Fab” DOI: 10.2210/pdb7ven/pdb; released on 14 Sep. 2022, incorporated herein by reference). The 2P39 FGF23 crystal structure and the 7VEN burosumab crystal structure were utilized for docking studies herein.

In short, protein-protein docking was performed using the FFT-based search algorithm implemented in Molecular Operating Environment “MOE” (2022.02). After molecule preparation (i.e., minimization, geometry, protonation, capping), all docking studies were initially performed with antibody and antigen molecules treated as rigid bodies. Conformational sampling of individual loops in the FGF23 antigen expected to be more flexible was performed using LowModeMD and regular MD. The models were first rendered into a coarse-grained (CG) residue-based representation in which each residue is composed of one to three “united-atom” beads following the scheme proposed by Basdevant et al (doi: 10.1021/jp0727190.). The CG representation includes the following energy components: van der Waals, electrostatic, and solvation via the Generalized Born Volume Integral (GB/VI) formalism.

In the docking process the antibody was considered as ‘ligand’ and the antigen FGF23 as ‘receptor’. The system was biased on the paratope side to consider published Kabat-defined burosumab CDRs as main interaction sites; i.e., Heavy Chain and Light Chain CDR1, CDR2, and CDR3 having sequences set forth in SEQ ID NO: 11, SEQ ID NO: 12, and SEQ ID NO: 13 (HCDR1, HCDR2, and HCDR3, respectively) and SEQ ID NO: 14, SEQ ID NO: 15, and SEQ ID NO: 16 (LCDR1, LCDR2, and LCDR3, respectively). In other words, the restrained ligand sites were fixed to burosumab CDRs whereas the epitopes were either considered as a whole (i.e., unbiased) or selected specifically based on HDX data. The hydrophobic patch potential was utilized throughout the search and refinement process. For pose selection, the threshold for retaining a pose as valid was set to pre-placement 10,000, placement as 1,000 and refinement as 100.

For docking purposes, the epitopes were selected based on empirical data from HDX-MS (Example 2 and Example 3 herein) in combination with (i) comparative analysis of cross-species reactivity studies using burosumab and FGF23 from different species, (ii) kinetics studies using sandwich SPR analysis of different burosumab mutational variants (Yamazaki et al., 2008), and/or (iii) patch analysis to evaluate alignment of hydrophobic and polar surfaces of FGF23 and burosumab that impact binding interactions.

With respect to the comparative analysis of burosumab binding across different species, it was observed that burosumab exhibits “similar high binding affinity (K_{D} 10-11 mol/L) to human, cynomolgus monkey and rabbit FGF23 (Study r-12-0326)” and cross-reactivity “with recombinant forms of monkey and human FGF23 but not mouse FGF23 (Study r-13-0026), and with plasma FGF23 from humans and monkeys but not dogs or rats (Study r-13-0027).” (See Center for Drug Evaluation and Research Application Number 7610680rig1s000, Multidisciplinary Review for Biologics License Application 761068, available at www.accessdata.fda.gov/drugsatfda_docs/nda/2018/761068Orig1s000MultidisciplineR.pdf). Accordingly, FGF23 amino acid sequences from human, cynomolgus monkey, rabbit, dog, and rat were aligned and overlayed with HDX-MS data to identify potential epitope residues or regions consistent across the burosumab cross-reactive species but not necessarily consistent with the non-cross-reactive species.

Findings from these various analyses were used to guide different biasing conditions in five separate docking searches (all FFT based and all biased on the antigen side) to generate diversity of poses and antigen-antibody contacts:
Search No. 1: Unbiased
Search No. 2: Biased using each HDX protected region 1, protected region 2, or protected region 3 considered separately
Search No. 3: Biased using all HDX protected regions (1, 2, and 3) together
Search No. 4: Biased using epitopes described in Yamazaki et al., 2008
Search No. 5: Biased using a shared epitope between HDX protected regions 1, 2, and 3 and Yamazaki et al., 2008

Each search run resulted in five different clusters, which were all further scrutinized for validation against energetics and empirical data (e.g., mutational data). The main requirement for a pose being considered a valid hit was approximately 30%-40% correlation to available HDX data, i.e., at least about 30% to about 40% (based on contacts within a 4.5 Angstrom sphere) of the total number of residues in the three epitopes suggested by HDX had to engage in at least one contact with any residue from the burosumab CDRs. The unbiased docking resulted in clusters with low overall correlation to the HDX data and mouse antibody bound epitopes found by Yamazaki et al. (doi:10.1359/JBMR.080417). Biased runs were more successful in this regard, with the best hit (i.e., highest correlation to HDX data) resulting from a run biased on the antigen side with the epitope reported from the mouse Ab in Yamazaki et al. (i.e., Search No. 4). Two poses were selected as leads based on the criteria set above: FGF23:Ab-Pose-1 (1st-preferred.moe) and FGF23:Ab-Pose-2 (2nd-preferred.moe). Several additional poses from docking runs 3, 4 and 5 were further evaluated.

Valid poses from all docking runs were scrutinized to identify common paratope positions. Information was combined with learnings from epitope and paratope mapping studies described in Example 2 and Example 3. Overall, 18 burosumab heavy chain and 18 burosumab light chain residues were identified as potential common paratope positions. Energetics of antigen-antibody contacts at these positions were evaluated for all positions. A summary of the modeling and docking runs is provided in Table 5.

TABLE 5
Modeling and docking runs summary

| | | | | Average | Utilized |
|---|---|---|---|---|---|
| | | | | % | in |
| Docking | Conforma- | | | epitope | muta- |
| Protocol | tional | Epitope | Output | cover- | tional |
| (MOE) | search | bias | poses | age | scans |
| Unbiased | No | No | Engages | 30~50 | |
| | | selection | protected | | |
| | | | region | | |
| | | | 2 or 3 | | |
| Biased | No | Protected | Engages | 60~75 | |
| | | region | protected | | |
| | | 1 only | region 1 | | |
| Biased | No | Protected | Engages | 60~75 | |
| | | region | protected | | |
| | | 2 only | region 2 | | |
| Biased | LowModeMD | Protected | Engages | 60~75 | |
| | | region | protected | | |
| | | 3 only | region 3 | | |
| Biased | No | Protected | Engages | ~50 | |
| | | region | protected | | |
| | | 2 & 3 | region | | |
| | | combined | 2 and 3 | | |
| Biased | LowModeMD | Protected | Engages | 20~30 | YES |
| | | region | all | | |
| | | 1 + 2 + 3 | protected | | |
| | | | regions | | |
| Biased | LowModeMD | Mu Ab | Engages | 20~30 | YES |
| | | | all | | |
| | | | protected | | |
| | | | regions | | |
| Biased | LowModeMD | Protected | Engages | 20~30 | YES |
| | | region | all | | |
| | | 1 + 2 + | protected | | |
| | | 3 + Mu Ab | regions | | |

### Example 5. Predictive Mutagenesis of Burosumab Based on Molecular Docking, HDX-MS Protection Mapping, and Comparative Mutation Analysis

Burosumab mutational variants were designed using predictive mutagenesis performed on common interacting burosumab positions across 5 averaged clusters. Common paratope positions (18 burosumab VH residues and 18 burosumab VL residues) were targeted for predictive mutagenesis, taking into consideration the valid poses from all docking searches which engaged at least about 60% of the HDX epitope residues. Positions and mutational variants were evaluated based on energy calculations and MM-based mutations and rotameric optimization (extended) within 4.5 angstrom from a mutation site.

The initial 1× scan using predictive mutagenesis resulted in 520 engineered FGF23-binding antibody designs, each comprising a single amino acid change relative to either the light chain of burosumab (SEQ ID NO: 2) or the heavy chain of burosumab (SEQ ID NO: 1). All 520 mutational variants were evaluated and screened according to the following criteria: dAffinity (ΔΔG (kcal/mol)) between −4.5 kcal/mol and 0.4 kcal/mol; dStability (ΔΔS (kcal/mol)) less than 3.0 kcal/mol; and redundant positions were re-assessed based on dSASA (difference in solvent accessible surface area) as an indicator of gain or loss of buried surface upon introducing the mutation. ΔΔG is a metric for predicting how a single-point mutation will affect protein stability. It is a measure of the change in energy between the folded and unfolded states and the change in ΔG folding when a point mutation is present. This metric is used as a predictor of whether a point mutation will be favorable in terms of protein stability. The more negative the value, the more stable the protein. ΔΔG values>0.5 suggest that a mutation would be destabilizing; 0.5>ΔΔG>−0.5 values suggest changes should be considered neutral or near neutral; and ΔΔG<−0.5: suggest that the mutation would lead to a more stable protein. ΔΔS, on the other hand, is a measure of vibration entropy difference. A positive value indicates increased flexibility, while a negative value indicates more rigid protein. It is used for predicting how a mutation will change the entropy of the protein. Water accessible surface area (“VdW SA”), water accessible surface area of hydrophobic atoms of the protein (“Hydrophobic SA”), and water accessible surface area of hydrophilic atoms of the protein (“Hydrophilic SA”) were also calculated. Table 6 provides a summary of the full 1× scan predictive mutagenesis results for burosumab light chain positions and Table 7 provides a summary of the full 1× scan predictive mutagenesis results for burosumab heavy chain positions.

From the full 1× scan results, 96 mutational variants were selected for further evaluation based on screening criteria described above to select for predicted improvements in affinity with minimal negative impacts to predicted stability or solvation. These 96 mutational variants are referred to herein as the burosumab variant or engineered FGF23-binding antibody “design panel 1.” Table 8 provides a summary of the 1× scan predictive mutagenesis results for burosumab light chain positions selected for the design panel 1 and Table 9 provides a summary of the 1× scan predictive mutagenesis results for burosumab heavy chain positions selected for the design panel 1.

TABLE 6
Burosumab light chain full 1X Scan predictive mutagenesis results

| LC | | | | | |
|---|---|---|---|---|---|
| Mutation | | | | | |
| (relative | | | | | |
| to SEQ ID | ΔΔG | ΔΔS | VdW | Hydrophobic | Hydrophilic |
| NO: 2) | (kcal/mol) | (kcal/mol) | SA (Å²) | SA (Å²) | SA (Å²) |
| G30A | −1.08 | −0.37 | −63.95 | 0.33 | −61.95 |
| G30C | 0.12 | 0.13 | −39.47 | 1.83 | −39.84 |
| G30D | 1.90 | 0.98 | −66.97 | −12.15 | −51.79 |
| G30E | 0.79 | 1.37 | −51.29 | 23.50 | −83.41 |
| G30F | 2.29 | 0.23 | 52.36 | 40.51 | 9.03 |
| G30G | 0.00 | 0.00 | 0.00 | 0.00 | 0.00 |
| G30H | 4.44 | 1.47 | −26.20 | 3.15 | −29.23 |
| G30I | 3.09 | −0.19 | −12.10 | 5.94 | −16.62 |
| G30K | 1.64 | 1.12 | 7.07 | −12.95 | 11.50 |
| G30L | 2.81 | 1.76 | −34.42 | 50.57 | −91.40 |
| G30M | 2.15 | 1.00 | −70.00 | 12.67 | −84.98 |
| G30N | 0.33 | 0.52 | −66.45 | −21.05 | −44.73 |
| G30P | −0.88 | 0.23 | −83.92 | −20.74 | −71.30 |
| G30Q | 2.33 | 1.21 | −29.03 | −9.02 | −18.24 |
| G30R | −1.55 | 0.54 | 68.26 | 22.89 | 45.58 |
| G30S | −0.56 | 0.30 | −46.30 | 7.83 | −53.98 |
| G30T | −3.88 | −0.30 | −57.13 | −5.29 | −51.15 |
| G30V | 1.20 | 0.13 | −57.42 | −16.26 | −41.77 |
| G30W | 2.87 | 0.76 | 13.94 | 39.40 | −29.14 |
| G30Y | 1.24 | 0.63 | −20.15 | 1.31 | −14.59 |
| I31A | 0.06 | 3.21 | −5.87 | −13.07 | 6.42 |
| I31C | −0.09 | 2.31 | 0.87 | −0.85 | 1.73 |
| I31D | −0.04 | 3.61 | −10.07 | −16.86 | 8.48 |
| I31E | −1.08 | 2.17 | −6.42 | −11.39 | 6.18 |
| I31F | −0.39 | 1.50 | −1.00 | 0.00 | 3.00 |
| I31G | −1.71 | 3.86 | −1.37 | −8.42 | 7.08 |
| I31H | −0.40 | 1.80 | −2.97 | −0.96 | 0.00 |
| I31I | 0.00 | 0.00 | 0.00 | 0.00 | 0.00 |
| I31K | −0.96 | 2.85 | 0.00 | −0.81 | 1.68 |
| I31L | −1.65 | 0.85 | −2.03 | 0.87 | −2.03 |
| I31M | −1.01 | 1.29 | −0.83 | −2.94 | 4.97 |
| I31N | −1.59 | 2.43 | 1.39 | −0.06 | 2.67 |
| I31P | −0.26 | 2.92 | −3.59 | −10.87 | 10.62 |
| I31Q | −1.00 | 2.08 | −1.40 | −2.25 | 2.03 |
| I31R | −0.52 | 2.68 | −4.48 | −2.34 | −2.45 |
| I31S | −0.49 | 3.45 | −10.23 | −14.00 | 4.69 |
| I31T | −1.27 | 2.90 | 4.88 | −1.15 | 6.87 |
| I31V | −0.28 | 2.24 | −1.85 | −2.59 | 1.61 |
| I31W | 2.17 | 5.12 | −10.09 | −3.42 | −4.34 |
| I31Y | 2.76 | 2.53 | −6.15 | −6.61 | 3.03 |
| Q29A | 3.58 | 1.21 | 8.36 | 16.36 | −11.58 |
| Q29C | 1.31 | 1.06 | 10.76 | 13.01 | −12.00 |
| Q29D | 1.19 | 1.62 | −0.45 | −0.49 | −2.31 |
| Q29E | 2.31 | 1.41 | 1.79 | 14.42 | −16.97 |
| Q29F | −2.13 | 0.79 | 5.77 | 78.62 | −66.50 |
| Q29G | 4.08 | 1.94 | 28.49 | 24.75 | 1.76 |
| Q29H | −2.04 | 0.80 | −4.70 | 3.37 | −15.47 |
| Q29I | 0.67 | 0.41 | 4.66 | 48.06 | −38.55 |
| Q29K | −1.10 | 0.68 | −5.52 | 24.69 | −26.92 |
| Q29L | −0.46 | −0.01 | −10.49 | 21.41 | −36.24 |
| Q29M | −0.94 | 0.48 | −12.24 | 46.42 | −52.52 |
| Q29N | −1.91 | 0.69 | 2.51 | 6.49 | −8.07 |
| Q29P | 6.13 | 2.91 | −6.95 | 52.50 | −33.64 |
| Q29Q | 0.00 | 0.00 | 0.00 | 0.00 | 0.00 |
| Q29R | −1.95 | 0.90 | 16.97 | −12.44 | 33.58 |
| Q29S | 2.14 | 1.39 | 9.74 | 12.69 | 0.26 |
| Q29T | 1.56 | 0.99 | 7.72 | 7.81 | 0.26 |
| Q29V | 1.17 | 0.43 | 8.79 | 31.73 | −21.59 |
| Q29W | −3.73 | 0.60 | 13.78 | 86.64 | −73.10 |
| Q29Y | −1.06 | 0.87 | 15.15 | 30.63 | −16.45 |
| S26A | 1.22 | 0.24 | 19.75 | 11.17 | 13.17 |
| S26C | −0.26 | 0.31 | 1.85 | −23.46 | 0.59 |
| S26D | 2.08 | 0.80 | −19.24 | −19.39 | −7.40 |
| S26E | 2.54 | 0.14 | −79.86 | −34.05 | −52.03 |
| S26F | 1.30 | 0.37 | 82.49 | 98.00 | −15.18 |
| S26G | −0.30 | 0.66 | −11.94 | −17.30 | −3.57 |
| S26H | 1.12 | 1.13 | 40.54 | −11.65 | 29.49 |
| S26I | −0.48 | −0.32 | 14.88 | 30.28 | −11.87 |
| S26K | 1.28 | 0.90 | 2.27 | −4.62 | 10.95 |
| S26L | −0.63 | 0.13 | 62.63 | 79.30 | −12.14 |
| S26M | 1.37 | 0.16 | 21.44 | 18.30 | 5.84 |
| S26N | −0.49 | 0.92 | 21.55 | −29.43 | 43.81 |
| S26P | 0.24 | 1.09 | −7.97 | 9.25 | −23.82 |
| S26Q | −0.49 | 0.45 | 20.00 | −25.33 | 40.32 |
| S26R | 1.83 | 0.11 | 23.69 | −5.99 | 29.98 |
| S26S | 0.00 | 0.00 | 0.00 | 0.00 | 0.00 |
| S26T | 1.32 | 0.59 | 19.80 | 32.21 | −10.00 |
| S26V | 1.20 | −0.06 | 37.12 | 33.29 | 4.36 |
| S26W | 1.14 | 0.66 | 54.57 | 77.14 | −19.85 |
| S26Y | 1.28 | 0.46 | 92.65 | 54.87 | 38.20 |
| S32A | 2.49 | 0.43 | −4.77 | 11.84 | −10.00 |
| S32C | −0.03 | 0.29 | −6.61 | −0.67 | −7.54 |
| S32D | −2.66 | 0.44 | −9.12 | −1.80 | 0.03 |
| S32E | −0.80 | 0.12 | −5.77 | −39.70 | 34.24 |
| S32F | −4.69 | −0.56 | −3.27 | −13.45 | 14.81 |
| S32G | 4.69 | 1.32 | 14.29 | 21.55 | −5.77 |
| S32H | −6.44 | 0.20 | −20.75 | −14.86 | −7.11 |
| S32I | −2.25 | −1.07 | −42.54 | −7.75 | −27.30 |
| S32K | 1.07 | 0.60 | −14.15 | −36.86 | 28.89 |
| S32L | −4.17 | −1.11 | −18.80 | 4.45 | −15.84 |
| S32M | −3.15 | −0.78 | −25.54 | −4.30 | −13.99 |
| S32N | −2.94 | 0.57 | 10.54 | 1.42 | 10.66 |
| S32P | 1.18 | 0.63 | −11.06 | 2.69 | −11.04 |
| S32Q | 0.11 | 0.79 | −23.04 | −60.57 | 35.10 |
| S32R | −1.79 | −0.62 | −9.67 | −43.08 | 35.07 |
| S32S | 0.00 | 0.00 | 0.00 | 0.00 | 0.00 |
| S32T | −0.85 | 0.02 | −11.88 | 2.31 | −8.79 |
| S32V | −0.42 | −0.60 | −18.06 | 2.26 | −17.52 |
| S32W | −6.27 | −0.45 | −18.02 | −23.70 | 8.10 |
| S32Y | −5.79 | −0.37 | 0.57 | −37.64 | 40.23 |
| S33A | 0.42 | 1.04 | −19.60 | 3.17 | −23.86 |
| S33C | −1.78 | 0.70 | −30.85 | 15.97 | −44.48 |
| S33D | −1.23 | 1.44 | −33.47 | 14.96 | −44.59 |
| S33E | −3.97 | 0.61 | −58.52 | −14.16 | −40.81 |
| S33F | −1.23 | 0.62 | −60.92 | 22.56 | −75.92 |
| S33G | 0.12 | 1.65 | −27.32 | 7.55 | −30.28 |
| S33H | −4.61 | 0.61 | −47.58 | 14.15 | −53.65 |
| S33I | −2.49 | −0.10 | −64.49 | −16.97 | −49.90 |
| S33K | −4.60 | 0.52 | 16.06 | 8.18 | 3.00 |
| S33L | −4.30 | −0.51 | −53.17 | −10.39 | −36.81 |
| S33M | −5.46 | −0.23 | −54.79 | −7.60 | −41.30 |
| S33N | −6.15 | 0.42 | −42.16 | 3.36 | −41.24 |
| S33P | 1.64 | 0.69 | −35.76 | 7.61 | −36.38 |
| S33Q | −2.86 | 0.58 | −49.64 | −11.74 | −29.14 |
| S33R | −4.65 | −0.09 | 3.22 | −18.33 | 15.86 |
| S33S | 0.00 | 0.00 | 0.00 | 0.00 | 0.00 |
| S33T | −1.88 | 0.39 | −44.64 | 2.43 | −33.14 |
| S33V | −1.11 | 0.12 | −59.36 | −11.72 | −49.40 |
| S33W | −6.76 | 0.88 | −45.79 | 27.60 | −73.54 |
| S33Y | −4.14 | −0.36 | −72.63 | 1.47 | −67.33 |
| S83A | 0.22 | 0.92 | −6.72 | 38.16 | −42.08 |
| S83C | 0.29 | 1.10 | 4.76 | −10.56 | −46.40 |
| S83D | −1.30 | 1.16 | −2.26 | −3.69 | −1.50 |
| S83E | −0.17 | 1.82 | 16.44 | −2.49 | 18.89 |
| S83F | 0.02 | 1.30 | 70.68 | 128.97 | −53.32 |
| S83G | −0.42 | 1.04 | −20.68 | −21.10 | −6.99 |
| S83H | 0.89 | 1.69 | 12.07 | −18.61 | −5.30 |
| S83I | −0.46 | 0.39 | −3.21 | 52.92 | −47.57 |
| S83K | 0.29 | 1.84 | 82.80 | 61.47 | 25.24 |
| S83L | −0.30 | 0.82 | 32.04 | 86.55 | −49.87 |
| S83M | −0.31 | 1.58 | 70.71 | 123.15 | −46.29 |
| S83N | 0.08 | 1.30 | −4.21 | −28.74 | 29.15 |
| S83P | 0.45 | 1.96 | 20.25 | 62.78 | −39.59 |
| S83Q | 0.35 | 1.53 | 12.03 | 8.05 | 6.29 |
| S83R | 0.58 | 0.90 | 44.01 | 18.56 | 26.23 |
| S83S | 0.00 | 0.00 | 0.00 | 0.00 | 0.00 |
| S83T | −0.10 | 0.75 | 17.37 | 29.78 | −11.74 |
| S83V | −0.26 | 0.55 | −2.82 | 53.07 | −49.37 |
| S83W | 0.78 | 1.29 | 57.36 | 116.69 | −52.42 |
| S83Y | 0.46 | 0.97 | 42.34 | 47.97 | 3.36 |
| T87A | −0.64 | 1.14 | −18.97 | 22.19 | −30.86 |
| T87C | 2.50 | 1.18 | −8.75 | 33.33 | −61.14 |
| T87D | 0.20 | 1.27 | −4.04 | 7.63 | −4.53 |
| T87E | 2.35 | 0.76 | 26.67 | −30.54 | 45.87 |
| T87F | −2.28 | 0.53 | −13.05 | 87.51 | −99.87 |
| T87G | 0.99 | 1.69 | −23.87 | 17.34 | −45.19 |
| T87H | −2.75 | 1.18 | −25.21 | −10.02 | −48.75 |
| T87I | −1.18 | 0.21 | −36.31 | 33.07 | −74.65 |
| T87K | −3.26 | −0.18 | −3.48 | 14.78 | −24.84 |
| T87L | 3.89 | 0.27 | 8.67 | 16.55 | −13.64 |
| T87M | −2.76 | 0.12 | −28.64 | 28.49 | −63.56 |
| T87N | −0.24 | 1.16 | −11.62 | 11.93 | −23.03 |
| T87P | −2.12 | 1.57 | −28.90 | 42.05 | −68.33 |
| T87Q | −4.00 | −0.27 | −56.56 | 9.56 | −63.09 |
| T87R | 0.49 | 0.25 | 97.79 | 11.60 | 79.36 |
| T87S | 2.44 | 1.43 | −27.13 | −8.85 | −17.39 |
| T87T | 0.00 | 0.00 | 0.00 | 0.00 | 0.00 |
| T87V | −0.23 | 0.29 | −40.10 | 26.71 | −65.20 |
| T87W | −1.79 | 1.03 | 1.07 | 60.34 | −58.28 |
| T87Y | 2.45 | 0.53 | 41.40 | −1.95 | 35.47 |

TABLE 7
Burosumab heavy chain full 1X Scan
predictive mutagenesis results

| HC | | | | | |
|---|---|---|---|---|---|
| Mutation | | | | | |
| (relative | | | | | |
| to SEQ ID | ΔΔG | ΔΔS | VdW | Hydrophobic | Hydrophilic |
| NO: 1) | (kcal/mol) | (kcal/mol) | SA (Å²) | SA (Å²) | SA (Å²) |
| A71A | 0.00 | 0.00 | 0.00 | 0.00 | 0.00 |
| A71C | −0.24 | 0.48 | −0.72 | −21.39 | 4.27 |
| A71D | 0.32 | 1.06 | 16.06 | −42.98 | 46.54 |
| A71E | 0.29 | 0.6 | 4.60 | −36.43 | 31.29 |
| A71F | −0.44 | −0.62 | −9.45 | −48.46 | 30.05 |
| A71G | −0.20 | 1.33 | 10.48 | −1.56 | 7.41 |
| A71H | −0.28 | 0.77 | −18.70 | −81.39 | 32.28 |
| A71I | −0.14 | −0.84 | −16.55 | −54.35 | 34.35 |
| A71K | 0.90 | −0.27 | −2.30 | −22.30 | 19.74 |
| A71L | −0.44 | −0.71 | 12.87 | −33.80 | 38.27 |
| A71M | 0.01 | 0.01 | −24.22 | −7.29 | −13.59 |
| A71N | −0.40 | 0.64 | 22.26 | −37.11 | 55.88 |
| A71P | −0.01 | 0.86 | 4.30 | −0.17 | 3.35 |
| A71Q | 0.11 | 0.45 | −13.00 | −15.99 | 4.53 |
| A71R | −0.11 | −0.47 | −43.20 | −25.75 | −8.14 |
| A71S | −0.40 | 0.68 | 1.48 | −10.95 | 7.87 |
| A71T | −0.25 | 0.40 | −1.24 | −6.85 | 3.63 |
| A71V | −0.23 | −0.11 | 2.42 | −26.12 | 28.84 |
| A71W | −0.25 | −0.71 | −8.16 | −37.88 | 21.91 |
| A71Y | −0.29 | −0.44 | −4.12 | −78.46 | 72.85 |
| D109A | 1.09 | 1.31 | −6.11 | −3.48 | 3.78 |
| D109C | 0.23 | 1.10 | −3.63 | −1.53 | 3.78 |
| D109D | 0.00 | 0.00 | 0.00 | 0.00 | 0.00 |
| D109E | 0.99 | 2.47 | −2.85 | −3.60 | 17.65 |
| D109F | −0.74 | 1.95 | −22.60 | −14.06 | 18.55 |
| D109G | 0.63 | 2.02 | −0.29 | −0.63 | 9.28 |
| D109H | 0.79 | 1.80 | −3.52 | −2.19 | 8.31 |
| D109I | 0.33 | 0.16 | −11.67 | −10.77 | 6.63 |
| D109K | 0.24 | 2.07 | −8.01 | 0.94 | 6.55 |
| D109L | 0.32 | 0.20 | −12.39 | −12.27 | 6.63 |
| D109M | −0.34 | 0.67 | −6.76 | −1.29 | 7.15 |
| D109N | −0.22 | 0.69 | −1.90 | −1.94 | 8.80 |
| D109P | 0.52 | 1.51 | 0.44 | −0.89 | 6.74 |
| D109Q | 0.74 | 1.17 | −9.52 | −4.71 | 3.29 |
| D109R | 2.29 | 0.82 | −9.27 | −5.93 | 8.72 |
| D109S | 0.56 | 0.86 | 0.06 | −1.02 | 9.28 |
| D109T | 0.36 | 1.09 | 6.75 | −0.48 | 10.97 |
| D109V | 0.37 | 0.38 | −6.17 | −10.64 | 9.77 |
| D109W | 0.25 | 1.59 | −6.48 | −9.44 | 6.56 |
| D109Y | 0.34 | 1.16 | −3.95 | −4.89 | 4.04 |
| D134A | 1.13 | 0.94 | −0.89 | −5.78 | 1.36 |
| D134C | 0.53 | 1.32 | −1.26 | −16.26 | 10.62 |
| D134D | 0.00 | 0.00 | 0.00 | 0.00 | 0.00 |
| D134E | −0.58 | 0.33 | 34.00 | −29.02 | 64.96 |
| D134F | −5.28 | −1.09 | −1.49 | −58.64 | 44.83 |
| D134G | 1.64 | 1.76 | 19.43 | 6.80 | 7.11 |
| D134H | −1.77 | 1.49 | −2.56 | −25.84 | −11.33 |
| D134I | −1.23 | −1.15 | −9.23 | 6.61 | −13.56 |
| D134K | −0.95 | 0.39 | 3.94 | −34.20 | 41.75 |
| D134L | −1.39 | −1.00 | −11.79 | 1.86 | −9.58 |
| D134M | −1.49 | 0.72 | −1.36 | 15.35 | −16.27 |
| D134N | −1.06 | 0.42 | −7.80 | −11.52 | 3.02 |
| D134P | 1.46 | 0.81 | 1.82 | 3.11 | −1.06 |
| D134Q | −0.56 | 0.73 | 26.93 | −38.77 | 66.46 |
| D134R | −3.70 | −0.60 | 6.07 | −30.65 | 42.00 |
| D134S | 1.08 | 1.60 | −6.50 | −14.68 | 1.74 |
| D134T | −0.07 | 0.61 | −1.67 | 2.87 | −4.67 |
| D134V | −0.55 | 0.08 | −10.96 | −7.73 | −5.71 |
| D134W | −4.83 | 0.33 | 8.34 | −2.89 | 7.63 |
| D134Y | −5.18 | −0.94 | −0.38 | −60.19 | 45.38 |
| H39A | 0.16 | 1.33 | 33.62 | 52.56 | −16.66 |
| H39C | 0.16 | 0.81 | 22.54 | −1.36 | 27.45 |
| H39D | 0.09 | 1.81 | 35.80 | 22.22 | 4.93 |
| H39E | −0.11 | 1.76 | 5.07 | 12.65 | −6.02 |
| H39F | −0.19 | 0.26 | 56.90 | 71.52 | −23.28 |
| H39G | 0.12 | 2.13 | 21.20 | 36.22 | −16.44 |
| H39H | 0.00 | 0.00 | 0.00 | 0.00 | 0.00 |
| H39I | 0.12 | −0.53 | 2.41 | 13.42 | −14.45 |
| H39K | 0.14 | 1.26 | 4.30 | 18.00 | −10.81 |
| H39L | 0.15 | 0.33 | 14.36 | 61.64 | −49.58 |
| H39M | −0.20 | 0.87 | 15.03 | 59.67 | −50.37 |
| H39N | 0.30 | 1.22 | 41.04 | 25.61 | 8.48 |
| H39P | −0.04 | 1.16 | 26.21 | −15.45 | 42.03 |
| H39Q | 0.12 | 0.76 | −12.43 | 13.28 | −25.82 |
| H39R | −0.06 | 0.29 | 23.54 | −1.64 | 22.15 |
| H39S | 0.09 | 1.46 | 15.65 | −4.91 | 22.49 |
| H39T | 0.10 | 0.67 | −1.67 | −2.52 | −0.55 |
| H39V | 0.02 | 0.03 | −5.04 | −2.30 | 0.92 |
| H39W | 0.04 | 0.28 | 28.21 | 33.64 | −4.07 |
| H39Y | −0.21 | 0.30 | 49.59 | 33.74 | 11.43 |
| I110A | 0.03 | 2.52 | 3.86 | −22.74 | 29.99 |
| I110C | −0.11 | 1.91 | 7.13 | −17.14 | 23.77 |
| I110D | −1.11 | 4.20 | 15.76 | −56.14 | 86.19 |
| I110E | −0.08 | 2.61 | −4.10 | −56.28 | 65.04 |
| I110F | −0.35 | 0.96 | −23.99 | −25.37 | 5.99 |
| I110G | 0.66 | 2.84 | 24.81 | −20.46 | 54.01 |
| I110H | −0.86 | 1.80 | −25.00 | −45.31 | 22.01 |
| I110I | 0.00 | 0.00 | 0.00 | 0.00 | 0.00 |
| I110K | −0.04 | 0.92 | −14.89 | −10.14 | 26.38 |
| I110L | −0.75 | 0.74 | −20.88 | −25.94 | 3.67 |
| I110M | −0.97 | 1.38 | −10.18 | −13.53 | 11.87 |
| I110N | −0.10 | 2.04 | 9.43 | 12.38 | 31.55 |
| I110P | 0.49 | 2.68 | −15.84 | −33.85 | 35.29 |
| I110Q | −1.22 | 1.59 | −21.18 | −62.41 | 63.88 |
| I110R | −0.24 | 1.42 | 5.96 | −48.18 | 53.29 |
| I110S | −0.94 | 2.52 | −2.78 | −33.44 | 45.29 |
| I110T | −0.20 | 1.50 | 9.40 | −31.40 | 44.59 |
| I110V | 0.22 | 1.91 | −7.61 | −14.51 | 5.64 |
| I110W | −3.68 | 1.40 | 14.00 | −3.39 | 24.52 |
| I110Y | −2.98 | 1.93 | 29.93 | −8.17 | 51.25 |
| N33A | −0.02 | 0.77 | −30.82 | 35.02 | −58.06 |
| N33C | 0.00 | 0.57 | −8.55 | 8.36 | −49.97 |
| N33D | −0.09 | 0.57 | 20.42 | 51.48 | −32.54 |
| N33E | −0.10 | 0.63 | −1.03 | −3.95 | 0.01 |
| N33F | −0.05 | 1.38 | 61.03 | 141.95 | −90.36 |
| N33G | −0.02 | 1.18 | −35.33 | 23.42 | −49.87 |
| N33H | −0.04 | 1.18 | 0.77 | 11.38 | −55.78 |
| N33I | −0.14 | 0.21 | 4.21 | 49.75 | −54.51 |
| N33K | 0.04 | 1.38 | 40.21 | 32.19 | −7.94 |
| N33L | −0.01 | 0.18 | 3.27 | 65.34 | −59.41 |
| N33M | 0.01 | 0.94 | 33.57 | 84.15 | −52.32 |
| N33N | 0.00 | 0.00 | 0.00 | 0.00 | 0.00 |
| N33P | −0.05 | 1.12 | −23.46 | 41.13 | −61.32 |
| N33Q | 0.01 | 0.64 | 18.36 | −8.17 | 22.72 |
| N33R | 0.12 | 1.04 | 62.06 | −4.16 | 62.99 |
| N33S | −0.03 | 0.82 | 7.00 | 55.14 | −51.31 |
| N33T | −0.08 | 0.58 | −24.68 | 36.49 | −70.86 |
| N33V | −0.09 | 0.13 | 5.70 | 36.04 | −29.54 |
| N33W | −0.02 | 1.33 | 62.07 | 108.12 | −62.31 |
| N33Y | −0.03 | 1.38 | 107.14 | 106.68 | 2.14 |
| N59A | 2.81 | 1.04 | 0.98 | −5.70 | 12.78 |
| N59C | 2.02 | 0.54 | 11.73 | 1.94 | 15.38 |
| N59D | 0.44 | 0.47 | −4.01 | −0.83 | 1.73 |
| N59E | 4.10 | 0.31 | −3.26 | −10.48 | 16.92 |
| N59F | 3.42 | −0.20 | −11.66 | −28.85 | 28.57 |
| N59G | 3.64 | 2.22 | −20.06 | −26.07 | 17.71 |
| N59H | 3.09 | 0.76 | −7.82 | −16.99 | 13.51 |
| N59I | 5.73 | 0.61 | −24.09 | −28.10 | 14.52 |
| N59K | 2.76 | 1.02 | 20.05 | 2.57 | 26.74 |
| N59L | 5.05 | −0.32 | −4.36 | −27.22 | 32.89 |
| N59M | 1.75 | 0.40 | −9.34 | −30.05 | 29.49 |
| N59N | 0.00 | 0.00 | 0.00 | 0.00 | 0.00 |
| N59P | 3.64 | 2.10 | −17.39 | −8.28 | 1.58 |
| N59Q | 4.08 | 0.98 | −9.24 | −33.40 | 30.19 |
| N59R | 5.05 | 1.24 | 77.11 | −71.11 | 147.91 |
| N59S | 1.49 | 1.09 | 2.34 | −4.04 | 12.33 |
| N59T | 3.18 | 1.08 | 3.67 | −5.68 | 17.74 |
| N59V | 3.51 | 0.32 | −1.53 | −16.96 | 17.42 |
| N59W | 1.71 | 0.31 | −17.88 | −29.79 | 20.45 |
| N59Y | 2.22 | −0.02 | −17.18 | −42.37 | 32.60 |
| N70A | −0.10 | 1.03 | −2.51 | 3.31 | −7.39 |
| N70C | −0.08 | 0.76 | 3.57 | 3.39 | −10.23 |
| N70D | 0.08 | 1.06 | −4.61 | −6.87 | 9.88 |
| N70E | 0.02 | 0.92 | −11.30 | −9.93 | −0.76 |
| N70F | −0.37 | −0.34 | −14.89 | 41.70 | −67.38 |
| N70G | −0.16 | 1.80 | 16.02 | 16.98 | −1.18 |
| N70H | −0.59 | 0.64 | −19.03 | 17.89 | −47.66 |
| N70I | −0.19 | −0.17 | −22.82 | −1.75 | −22.68 |
| N70K | −0.79 | 0.46 | −12.35 | −33.00 | 14.18 |
| N70L | −0.09 | 0.20 | −20.21 | 2.14 | −16.58 |
| N70M | −0.12 | 0.31 | −17.79 | 20.53 | −40.15 |
| N70N | 0.00 | 0.00 | 0.00 | 0.00 | 0.00 |
| N70P | −0.12 | 1.10 | −38.08 | −11.79 | −26.89 |
| N70Q | 0.10 | 0.61 | −9.05 | −6.74 | 3.05 |
| N70R | −0.82 | −0.13 | −30.02 | −43.49 | 12.01 |
| N70S | −0.09 | 1.27 | −26.89 | −13.88 | −8.22 |
| N70T | −0.01 | 0.95 | −24.39 | −5.99 | −17.68 |
| N70V | −0.02 | 0.09 | −25.15 | −5.26 | −14.47 |
| N70W | 0.00 | −0.79 | −19.19 | 17.58 | −41.18 |
| N70Y | −0.59 | −0.26 | −13.76 | 3.60 | −18.76 |
| Q72A | 0.81 | 1.11 | 43.69 | 69.49 | −29.12 |
| Q72C | 0.26 | 1.01 | 1.45 | 11.33 | −42.15 |
| Q72D | 0.22 | 1.28 | 9.09 | 1.52 | −1.98 |
| Q72E | 1.86 | 0.94 | 12.44 | 18.24 | −12.71 |
| Q72F | 0.73 | 0.19 | 12.64 | 46.68 | −43.84 |
| Q72G | 0.84 | 1.31 | 36.98 | 56.63 | −27.59 |
| Q72H | −0.13 | 0.85 | −19.50 | −13.97 | −19.77 |
| Q72I | 0.67 | 0.09 | 18.98 | 53.40 | −38.11 |
| Q72K | −0.57 | 1.00 | −7.25 | 17.06 | −26.61 |
| Q72L | 0.44 | −0.06 | −8.56 | 27.95 | −38.60 |
| Q72M | 0.05 | 0.37 | −4.86 | 20.97 | −34.50 |
| Q72N | 1.36 | 1.19 | −7.97 | −19.87 | 3.40 |
| Q72P | 0.78 | 1.02 | 27.69 | 50.65 | −30.55 |
| Q72Q | 0.00 | 0.00 | 0.00 | 0.00 | 0.00 |
| Q72R | −1.50 | −0.07 | −42.84 | 0.77 | −48.40 |
| Q72S | 0.81 | 1.26 | 36.22 | 33.43 | −0.29 |
| Q72T | 0.83 | 0.83 | 21.49 | 22.57 | −4.67 |
| Q72V | 0.84 | 0.39 | 16.87 | 47.51 | −36.60 |
| Q72W | 0.81 | 0.62 | 21.38 | 63.16 | −47.99 |
| Q72Y | 0.81 | 0.01 | 4.42 | 27.35 | −25.52 |
| Q75A | 0.67 | 1.25 | 10.50 | 46.35 | −37.75 |
| Q75C | 0.89 | 1.02 | 0.03 | 16.79 | −42.18 |
| Q75D | 0.93 | 1.56 | 12.95 | 16.57 | −2.77 |
| Q75E | 0.27 | 1.12 | 4.40 | 23.65 | −16.61 |
| Q75F | 0.16 | 0.06 | −44.15 | 7.99 | −56.72 |
| Q75G | 0.71 | 1.44 | −6.84 | 15.41 | −29.12 |
| Q75H | 0.49 | 0.83 | −40.66 | −4.81 | −44.63 |
| Q75I | 0.50 | 0.37 | −13.64 | 34.56 | −49.02 |
| Q75K | −0.01 | 1.31 | −51.15 | −22.59 | −35.80 |
| Q75L | 0.36 | 0.27 | −9.89 | 34.92 | −47.72 |
| Q75M | −0.08 | 0.09 | −13.78 | 36.04 | −48.31 |
| Q75N | 0.99 | 1.18 | 3.23 | 6.29 | −1.86 |
| Q75P | 0.68 | 1.60 | 23.11 | 76.20 | −48.18 |
| Q75Q | 0.00 | 0.00 | 0.00 | 0.00 | 0.00 |
| Q75R | −1.13 | 0.02 | −60.61 | −27.51 | −32.47 |
| Q75S | 0.67 | 1.38 | 14.56 | 21.14 | −7.46 |
| Q75T | −0.06 | 0.97 | 5.14 | 46.41 | −41.22 |
| Q75V | 0.69 | 0.74 | 7.10 | 54.24 | −48.08 |
| Q75W | −1.42 | −0.54 | −37.32 | −5.77 | −32.09 |
| Q75Y | 0.35 | 0.32 | −31.53 | 3.48 | −45.08 |
| S65A | −0.12 | 0.53 | 13.08 | 23.81 | −9.53 |
| S65C | −4.12 | −0.13 | −8.32 | −23.34 | −7.17 |
| S65D | −4.17 | 0.26 | 2.28 | −6.78 | 8.01 |
| S65E | −1.10 | 0.39 | −2.60 | −0.24 | −4.82 |
| S65F | −4.44 | −1.01 | 3.16 | 20.91 | −16.75 |
| S65G | 2.62 | 1.27 | 9.93 | −18.07 | 18.74 |
| S65H | −5.02 | 0.05 | −3.22 | −29.17 | 17.09 |
| S65I | −2.78 | −1.04 | 15.97 | 14.52 | −2.59 |
| S65K | 3.80 | 1.97 | 89.30 | 36.14 | 50.78 |
| S65L | 0.34 | 0.20 | 51.12 | 43.52 | 10.62 |
| S65M | −3.92 | −1.15 | −17.54 | −9.74 | −12.34 |
| S65N | −1.48 | 0.48 | 3.19 | −19.58 | 24.87 |
| S65P | −5.04 | 0.17 | −1.09 | 24.81 | −31.29 |
| S65Q | −2.39 | 0.09 | 4.90 | −37.41 | 42.36 |
| S65R | 2.04 | 2.07 | 108.27 | −10.53 | 112.57 |
| S65S | 0.00 | 0.00 | 0.00 | 0.00 | 0.00 |
| S65T | −4.54 | 0.04 | 0.58 | 15.66 | −25.65 |
| S65V | −2.04 | −0.87 | 59.01 | 17.96 | 28.42 |
| S65W | −6.13 | −0.30 | 30.56 | 54.00 | −18.70 |
| S65Y | −0.49 | 0.49 | 131.42 | 57.12 | 65.45 |
| S67A | 2.92 | 0.61 | 19.59 | −6.66 | 24.04 |
| S67C | 1.46 | 0.49 | 64.67 | −1.73 | 36.81 |
| S67D | −2.53 | −0.28 | 26.94 | 4.50 | 17.83 |
| S67E | 0.08 | −0.45 | 26.27 | 13.43 | 7.14 |
| S67F | −1.50 | −0.50 | 106.56 | 43.44 | 49.84 |
| S67G | 3.79 | 1.40 | 42.47 | 0.89 | 35.01 |
| S67H | −1.22 | −0.33 | 83.94 | −7.51 | 82.67 |
| S67I | 4.55 | −0.82 | 26.00 | −7.76 | 21.19 |
| S67K | −0.36 | 0.35 | 78.41 | −4.44 | 67.49 |
| S67L | −0.58 | −1.05 | 81.94 | 14.21 | 57.62 |
| S67M | −1.84 | −0.62 | 36.69 | −10.26 | 39.68 |
| S67N | −0.03 | 0.31 | 45.11 | −8.29 | 45.32 |
| S67P | 3.15 | 1.14 | 17.80 | −15.72 | 32.07 |
| S67Q | −3.56 | −0.14 | 0.39 | −11.02 | 5.48 |
| S67R | −4.48 | −0.10 | −22.51 | −34.67 | 2.34 |
| S67S | 0.00 | 0.00 | 0.00 | 0.00 | 0.00 |
| S67T | 0.10 | 0.09 | 20.02 | −0.42 | 14.33 |
| S67V | 5.45 | −0.20 | 6.78 | −9.75 | 13.56 |
| S67W | −0.22 | 0.41 | 108.15 | 70.33 | 34.17 |
| S67Y | −2.41 | −0.40 | 12.16 | −6.81 | 8.35 |
| S69A | 0.32 | 0.24 | 7.58 | 0.23 | 6.83 |
| S69C | 0.34 | 0.59 | 4.35 | −24.72 | −1.95 |
| S69D | −0.38 | 0.79 | −1.82 | −8.07 | 12.29 |
| S69E | 0.57 | 0.86 | −36.47 | −30.01 | −0.83 |
| S69F | −2.00 | −1.23 | −70.46 | −5.67 | −58.52 |
| S69G | 2.51 | 0.93 | 4.56 | −5.99 | 7.06 |
| S69H | −2.51 | 0.15 | −18.70 | −33.09 | 4.06 |
| S69I | 0.25 | −0.50 | −25.49 | 4.41 | −27.92 |
| S69K | −0.58 | 0.03 | −64.03 | −25.62 | −36.75 |
| S69L | 0.50 | −0.54 | −33.67 | −18.55 | −18.57 |
| S69M | −0.89 | −0.13 | −56.36 | −20.97 | −26.61 |
| S69N | −0.53 | 0.79 | 3.79 | −31.49 | 39.29 |
| S69P | −0.06 | 0.75 | 1.88 | 0.61 | 2.65 |
| S69Q | −0.34 | 0.48 | −15.74 | −20.27 | 5.48 |
| S69R | −0.77 | 0.06 | −59.67 | −4.42 | −45.82 |
| S69S | 0.00 | 0.00 | 0.00 | 0.00 | 0.00 |
| S69T | 0.07 | 0.15 | 1.64 | −9.94 | 19.53 |
| S69V | 0.38 | −0.26 | 13.93 | 12.39 | −5.67 |
| S69W | −2.16 | −1.49 | −46.54 | −47.78 | 12.09 |
| S69Y | −1.47 | −1.26 | −67.28 | −19.66 | −37.97 |
| T30A | 0.00 | 0.61 | −11.74 | 11.77 | −24.87 |
| T30C | 0.00 | 0.71 | −8.14 | −14.15 | −37.98 |
| T30D | −0.02 | 1.30 | −2.98 | −12.93 | 3.72 |
| T30E | −0.01 | 1.23 | 15.27 | −8.84 | 18.91 |
| T30F | 0.00 | 1.13 | 46.05 | 98.24 | −54.98 |
| T30G | 0.00 | 0.76 | −18.68 | −13.38 | −4.36 |
| T30H | 0.00 | 1.68 | 18.63 | −10.90 | −3.27 |
| T30I | 0.00 | 0.49 | 39.16 | 82.28 | −43.13 |
| T30K | 0.00 | 1.18 | 28.72 | 45.67 | −21.13 |
| T30L | 0.00 | 0.35 | 29.04 | 78.75 | −49.35 |
| T30M | 0.00 | 0.82 | 25.87 | 70.83 | −47.96 |
| T30N | 0.01 | 1.10 | 16.14 | −17.42 | 24.76 |
| T30P | 0.00 | 1.22 | 5.32 | 39.38 | −33.98 |
| T30Q | 0.00 | 0.85 | 18.27 | −11.79 | 28.08 |
| T30R | 0.00 | 0.76 | 22.62 | 18.79 | −4.70 |
| T30S | 0.00 | 0.86 | −4.91 | −3.81 | −1.05 |
| T30T | 0.00 | 0.00 | 0.00 | 0.00 | 0.00 |
| T30V | 0.00 | 0.45 | 17.71 | 58.37 | −41.21 |
| T30W | −0.01 | 1.48 | 114.88 | 123.88 | −9.38 |
| T30Y | 0.00 | 1.47 | 116.20 | 94.79 | 18.90 |
| T32A | 0.19 | 0.38 | −6.18 | −37.56 | 20.12 |
| T32C | 0.20 | 0.40 | −11.53 | −50.81 | 20.13 |
| T32D | 0.12 | 0.78 | 14.57 | −24.89 | 33.84 |
| T32E | 0.16 | 0.93 | 26.17 | −38.13 | 59.09 |
| T32F | 0.13 | −0.15 | 28.84 | 25.65 | 0.78 |
| T32G | 0.19 | 1.08 | −1.36 | −36.58 | 24.21 |
| T32H | 0.07 | 0.74 | 10.44 | −17.33 | 10.59 |
| T32I | 0.11 | −0.25 | −15.44 | −27.53 | 3.90 |
| T32K | 0.37 | 0.61 | −26.14 | −49.64 | 20.11 |
| T32L | 0.10 | −0.10 | 25.37 | 17.42 | 2.58 |
| T32M | 0.14 | 0.28 | 31.60 | −11.72 | 44.26 |
| T32N | 0.16 | 0.51 | −17.14 | −67.87 | 45.79 |
| T32P | 0.29 | −0.72 | −19.41 | −77.33 | 50.21 |
| T32Q | 0.31 | 0.31 | −19.90 | −72.23 | 41.94 |
| T32R | 0.18 | 0.72 | 63.76 | −22.60 | 80.26 |
| T32S | 0.20 | 0.53 | −16.27 | −47.27 | 24.69 |
| T32T | 0.00 | 0.00 | 0.00 | 0.00 | 0.00 |
| T32V | 0.13 | −0.21 | −16.86 | −32.39 | 11.90 |
| T32W | 0.16 | 0.22 | 37.36 | 36.07 | 3.94 |
| T32Y | 0.07 | −0.13 | 27.88 | 4.80 | 15.38 |
| T68A | −0.18 | 0.57 | −1.00 | −13.74 | 13.13 |
| T68C | −0.69 | 0.70 | −0.05 | −41.46 | 0.84 |
| T68D | −0.22 | 1.72 | −4.80 | −54.49 | 44.08 |
| T68E | −0.30 | 0.81 | −8.59 | −30.67 | 12.93 |
| T68F | 0.45 | −0.74 | −8.40 | −1.89 | −6.36 |
| T68G | −1.16 | 1.00 | −1.72 | −42.39 | 37.06 |
| T68H | −0.33 | 1.13 | 3.11 | −59.32 | 30.18 |
| T68I | 0.31 | −0.19 | −20.19 | −6.23 | −13.07 |
| T68K | −0.12 | 1.35 | 27.97 | −6.20 | 38.22 |
| T68L | 0.29 | −0.01 | −18.31 | −25.44 | 8.66 |
| T68M | −0.59 | 0.24 | −5.95 | 5.21 | −14.50 |
| T68N | −0.69 | 0.88 | −10.74 | −45.81 | 36.93 |
| T68P | 0.18 | 1.61 | 4.02 | 8.35 | −9.56 |
| T68Q | −0.39 | 0.86 | 1.34 | −42.99 | 45.85 |
| T68R | −1.44 | 1.39 | 21.36 | −20.24 | 30.08 |
| T68S | −0.31 | 0.79 | 7.63 | −35.17 | 42.84 |
| T68T | 0.00 | 0.00 | 0.00 | 0.00 | 0.00 |
| T68V | 0.46 | 0.08 | −10.52 | −8.51 | 0.07 |
| T68W | 0.47 | −0.45 | −6.62 | 23.33 | −28.66 |
| T68Y | 0.16 | −0.57 | −9.52 | −2.27 | −5.81 |
| V111A | 3.33 | 2.33 | −18.99 | −31.37 | 15.52 |
| V111C | 1.74 | 2.65 | −5.84 | −0.77 | −4.05 |
| V111D | 4.78 | 2.90 | −2.37 | 0.06 | 2.36 |
| V111E | 5.59 | 3.19 | −13.41 | −1.95 | −9.26 |
| V111F | 1.40 | 2.91 | −21.61 | −18.60 | −3.79 |
| V111G | 5.03 | 2.67 | 3.57 | −1.87 | 5.25 |
| V111H | 1.11 | 1.35 | 8.97 | 14.37 | −2.44 |
| V111I | 2.10 | 1.18 | 23.09 | −26.50 | 46.90 |
| V111K | −0.12 | 1.41 | −3.09 | 0.53 | −5.32 |
| V111L | 2.81 | 0.89 | −0.93 | 0.46 | 2.65 |
| V111M | 0.87 | 1.80 | −10.16 | −2.62 | −5.17 |
| V111N | 1.05 | 2.83 | −0.92 | 0.77 | −1.10 |
| V111P | 1.01 | 1.72 | −0.59 | 0.25 | 1.46 |
| V111Q | 0.83 | 2.23 | −10.49 | −3.02 | −5.92 |
| V111R | −1.55 | 1.47 | −9.66 | −2.37 | −6.33 |
| V111S | 2.86 | 2.08 | 3.15 | 4.38 | −0.72 |
| V111T | 3.45 | 1.54 | 13.58 | −31.54 | 48.63 |
| V111V | 0.00 | 0.00 | 0.00 | 0.00 | 0.00 |
| V111W | −0.88 | 2.30 | −36.42 | −27.02 | −7.00 |
| V111Y | 1.01 | 2.62 | −38.71 | −34.80 | 0.84 |
| Y40A | 1.39 | 1.09 | 17.94 | 12.69 | −3.82 |
| Y40C | 0.57 | 1.07 | 8.41 | 3.72 | −4.83 |
| Y40D | −2.54 | 1.59 | 25.93 | 10.84 | 6.17 |
| Y40E | −1.15 | 1.24 | 15.75 | 6.86 | −0.69 |
| Y40F | −0.27 | 0.03 | 3.07 | 4.87 | −4.15 |
| Y40G | 1.36 | 2.08 | 32.33 | 20.21 | −5.50 |
| Y40H | −0.79 | 0.87 | −3.02 | −4.37 | −3.48 |
| Y40I | −2.73 | −0.32 | 19.63 | 12.59 | 3.12 |
| Y40K | −1.63 | 0.63 | −3.55 | −1.51 | −2.20 |
| Y40L | −3.11 | −0.43 | 14.84 | 7.13 | 1.10 |
| Y40M | −1.14 | −0.19 | 20.57 | 10.21 | −1.65 |
| Y40N | 0.38 | 1.01 | 4.80 | −0.24 | 6.22 |
| Y40P | −1.54 | 1.35 | 18.71 | 9.01 | −0.62 |
| Y40Q | −0.63 | 0.78 | 15.18 | 8.32 | −3.20 |
| Y40R | −3.05 | −0.29 | 2.89 | 4.69 | −6.25 |
| Y40S | −0.94 | 1.21 | 26.63 | 14.15 | 2.19 |
| Y40T | −3.15 | 0.33 | 20.04 | 6.19 | 12.18 |
| Y40V | −1.94 | 0.02 | 15.85 | 11.13 | 2.19 |
| Y40W | −2.56 | −0.51 | −1.33 | 4.76 | −3.41 |
| Y40Y | 0.00 | 0.00 | 0.00 | 0.00 | 0.00 |

TABLE 8
Burosumab light chain design panel 1

| Mutation (ASN#) | | |
|---|---|---|
| (relative to SEQ ID NO: 2) | ΔΔG (kcal/mol) | ΔΔS (kcal/mol) |
| G30A | −1.1 | −0.4 |
| G30P | −0.9 | 0.2 |
| G30S | −0.6 | 0.3 |
| I31E | −1.1 | 2.2 |
| I31K | −1.0 | 2.8 |
| I31M | −1.0 | 1.3 |
| I31Q | −1.0 | 2.1 |
| I31R | −0.5 | 2.7 |
| I31S | −0.5 | 3.5 |
| I31T | −1.3 | 2.9 |
| Q29K | −1.1 | 0.7 |
| Q29M | −0.9 | 0.5 |
| Q29Y | −1.1 | 0.9 |
| S26I | −0.5 | −0.3 |
| S26L | −0.6 | 0.1 |
| S26N | −0.5 | 0.9 |
| S26Q | −0.5 | 0.5 |
| S32D | −2.6 | 0.4 |
| S32F | −4.7 | −0.6 |
| S32I | −2.2 | −1.1 |
| S32L | −4.2 | −1.1 |
| S32M | −3.1 | −0.8 |
| S32R | −1.8 | −0.6 |
| S32T | −0.8 | 0.0 |
| S32V | −0.4 | −0.6 |
| S33D | −1.2 | 1.4 |
| S33F | −1.2 | 0.6 |
| S33L | −4.3 | −0.5 |
| S33V | −1.1 | 0.1 |
| S83D | −1.3 | 1.2 |
| T87A | −0.6 | 1.1 |
| T87I | −1.2 | 0.2 |

TABLE 9
Burosumab heavy chain design panel 1

| Mutation (ASN#) | | |
|---|---|---|
| (relative to SEQ ID NO: | | |
| 1) | ΔΔG (kcal/mol) | ΔΔS (kcal/mol) |
| A71F | −0.4 | −0.6 |
| A71I | −0.1 | −0.8 |
| A71L | −0.4 | −0.7 |
| A71W | −0.2 | −0.7 |
| D109F | −0.7 | 2.0 |
| D134E | −0.6 | 0.3 |
| D134F | −5.3 | −1.1 |
| D134I | −1.2 | −1.1 |
| D134K | −1.0 | 0.4 |
| D134L | −1.4 | −1.0 |
| D134M | −1.5 | 0.7 |
| D134N | −1.1 | 0.4 |
| D134Q | −0.6 | 0.7 |
| D134R | −3.7 | −0.6 |
| D134V | −0.5 | 0.1 |
| D134Y | −5.2 | −0.9 |
| H39I | 0.1 | −0.5 |
| I110D | −1.1 | 4.2 |
| I110H | −0.9 | 1.8 |
| I110L | −0.7 | 0.7 |
| I110M | −1.0 | 1.4 |
| I110Q | −1.2 | 1.6 |
| I110S | −0.9 | 2.5 |
| N70H | −0.6 | 0.6 |
| N70K | −0.8 | 0.5 |
| N70R | −0.8 | −0.1 |
| N70W | 0.0 | −0.8 |
| N70Y | −0.6 | −0.3 |
| Q72K | −0.6 | 1.0 |
| Q72R | −1.5 | −0.1 |
| Q75R | −1.1 | 0.0 |
| Q75W | −1.4 | −0.5 |
| S65E | −1.1 | 0.4 |
| S65F | −4.4 | −1.0 |
| S65I | −2.8 | −1.0 |
| S65M | −3.9 | −1.2 |
| S65N | −1.5 | 0.5 |
| S65V | −2.0 | −0.9 |
| S65Y | −0.5 | 0.5 |
| S67F | −1.5 | −0.5 |
| S67H | −1.2 | −0.3 |
| S67I | 4.6 | −0.8 |
| S67L | −0.6 | −1.1 |
| S67M | −1.8 | −0.6 |
| S69F | −2.0 | −1.2 |
| S69I | 0.2 | −0.5 |
| S69K | −0.6 | 0.0 |
| S69L | 0.5 | −0.5 |
| S69M | −0.9 | −0.1 |
| S69N | −0.5 | 0.8 |
| S69R | −0.8 | 0.1 |
| S69W | −2.2 | −1.5 |
| S69Y | −1.5 | −1.3 |
| T32P | 0.3 | −0.7 |
| T68F | 0.4 | −0.7 |
| T68G | −1.2 | 1.0 |
| T68M | −0.6 | 0.2 |
| T68N | −0.7 | 0.9 |
| T68R | −1.4 | 1.4 |
| T68Y | 0.2 | −0.6 |
| V111W | −0.9 | 2.3 |
| Y40E | −1.2 | 1.2 |
| Y40H | −0.8 | 0.9 |
| Y40M | −1.1 | −0.2 |
| Y40Q | −0.6 | 0.8 |
| Y40S | −0.9 | 1.2 |

### Example 6. Engineered FGF23-Binding Antibody Rational Design Panel 1 Based on Molecular Docking Studies

Table 10 shows 96 engineered FGF23-binding antibody designs from the 1× Scan. Each variant is labeled with a sequential Engineered FGF23-binding Antibody Structure ID numbered from 001 through 096 and has one amino acid change relative to the parental burosumab heavy chain amino acid sequence (SEQ ID NO: 1) or the parental burosumab light chain amino acid sequence (SEQ ID NO: 2). No combined amino acid changes relative to the parental sequences were included in the design panel 1, and no variants were included having amino acid changes on more than one of the light or heavy chain. In Table 10, all amino acid numbering is presented according to the ASN #system.

TABLE 10
Design panel 1 of Engineered FGF23-binding antibodies

| Engineered | Light Chain | | |
|---|---|---|---|
| FGF23-Binding | Mutations (ASN#) | Engineered FGF23- | |
| Antibody | (relative to SEQ ID | Binding Antibody | Heavy Chain Mutations |
| Structure ID | NO: 2) | Structure ID | (relative to SEQ ID NO: 1) |
| UGX001 | Q29F | UGX044 | T30Y |
| UGX002 | Q29R | UGX045 | T32K |
| UGX003 | G30D | UGX046 | H39K |
| UGX004 | G30Q | UGX047 | H39Y |
| UGX005 | G30R | UGX048 | Y40D |
| UGX006 | G30Y | UGX049 | Y40L |
| UGX007 | S32D | UGX050 | Y40R |
| UGX008 | S32F | UGX051 | N59E |
| UGX009 | S32R | UGX052 | N59Q |
| UGX010 | S32Y | UGX053 | N59R |
| UGX011 | S33F | UGX054 | S65E |
| UGX012 | S33Y | UGX055 | S65F |
| UGX013 | D58E | UGX056 | S65I |
| UGX014 | D58Q | UGX057 | S65Q |
| UGX015 | D58R | UGX058 | S65R |
| UGX016 | D58Y | UGX059 | S65Y |
| UGX017 | S68E | UGX060 | S67E |
| UGX018 | S68Q | UGX061 | S67F |
| UGX019 | S68R | UGX062 | S67R |
| UGX020 | S68Y | UGX063 | S67Y |
| UGX021 | S69R | UGX064 | T68F |
| UGX022 | S69Y | UGX065 | T68R |
| UGX023 | L70E | UGX066 | S69F |
| UGX024 | L70Y | UGX067 | S69R |
| UGX025 | E71R | UGX068 | S69Y |
| UGX026 | E71Y | UGX069 | N70E |
| UGX027 | S83E | UGX070 | N70R |
| UGX028 | S83R | UGX071 | N70Y |
| UGX029 | S83Y | UGX072 | A71F |
| UGX030 | T87R | UGX073 | A71R |
| UGX031 | T87Y | UGX074 | A71Y |
| UGX032 | F109E | UGX075 | Q72L |
| UGX033 | F109R | UGX076 | Q72R |
| UGX034 | N110E | UGX077 | Q72Y |
| UGX035 | N11OF | UGX078 | Q75E |
| UGX036 | N110Q | UGX079 | Q75R |
| UGX037 | N110R | UGX080 | Q75Y |
| UGX038 | N110Y | UGX081 | D109F |
| UGX039 | D135F | UGX082 | D109R |
| UGX040 | D135R | UGX083 | D109Y |
| UGX041 | D135Y | UGX084 | I110E |
| UGX042 | Y136E | UGX085 | I110F |
| UGX043 | Y136R | UGX086 | I110Y |
| | | UGX087 | V111E |
| | | UGX088 | V111F |
| | | UGX089 | V111L |
| | | UGX090 | V111Q |
| | | UGX091 | V111R |
| | | UGX092 | D134E |
| | | UGX093 | D134F |
| | | UGX094 | D134K |
| | | UGX095 | D134R |
| | | UGX096 | D134Y |

### Example 7. Structural Evaluation of Burosumab-FGF23 Interaction

To further characterize the human FGF23-burosumab interaction, x-ray crystallographic study was carried out using fully glycosylated human FGF23 (R179Q) from HEK293S cell supernatant in complex with burosumab Fab fragments also expressed in HEK293S cells. Burosumab IgG heavy and light chains were subjected to papain digestion to separate Fc and Fab fragments, then Protein A to isolate Fab fragments of approximately 50 kDa, according to routine methods (Pierce™ Fab Preparation Kit, Thermo Scientific™, cat #!44985). Fully glycosylated human FGF23 (R179Q) and burosumab Fab fragments were incubated on ice for more than one hour (1:0.8 molar ratio) and the stable complex was isolated by size exclusion chromatography (SEC) followed by evaluation by SDS-PAGE. SEC fractions containing the complex were pooled, concentrated, and stored at −80° C.

Briefly, crystallization trials were performed in 96-well vapor diffusion sifting drop plates by mixing 200 nl of protein solution (4.5 mg/ml in 25 mM Hepes pH 7.5 and 150 mM NaCl) and 200 nl well solution (0.2 M ammonium citrate dibasic and 20% (w/v) PEG 3350). Diffraction data was collected from a single crystal at the Advanced Photon Source (APS) synchrotron and processed using autoPROC (autoProc, XDS, POINTLESS, AIMLESS, CCP4, and STARANISO) (see Vonrhein C, Flensburg C, Keller P, Sharff A, Smart O, Paciorek W, Womack T, Bricogne G. Data processing and analysis with the autoPROC toolbox. Acta Crystallogr D Biol Crystallogr. 2011 April; 67(Pt 4):293-302. [doi:10.1107/S0907444911007773]).

The phase solution was obtained using Phaser (CCP4) (see J. Agirre et al. Acta. Cryst. D79, 449-461 (2023) “The CCP4 suite: integrative software for macromolecular crystallography” [doi:10.1107/52059798323003595]) with FGF23 and burosumab (PDB IDs 2P32 and 7VEN) as models for molecular replacement. Refinement and model building were performed with RECMAC, BUSTER and COOT (see J. Agirre et al.; Bricogne G., Blanc E., Brandl M., Flensburg C., Keller P., Paciorek W., Roversi P, Sharff A., Smart O. S., Vonrhein C., Womack T. O. (2017). Cambridge. United Kingdom: Global Phasing Ltd.; and Emslev P, Lohkamp B. Scott W G. Cowtan K. Features and development of Coot. Acta Crystallogr. D. Biol. Crystallogr. 2010; 66:486-501. [doi:10.1107/S0907444910007493]).

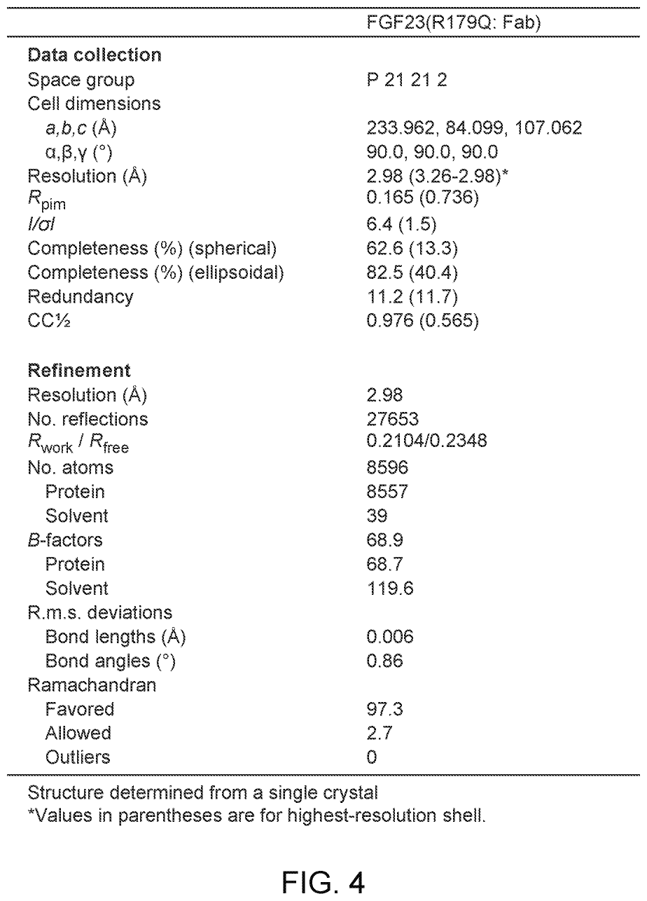

Data collection and refinement statistics for the 3 Å complex structure solved by molecular replacement are provided in FIG. 4.

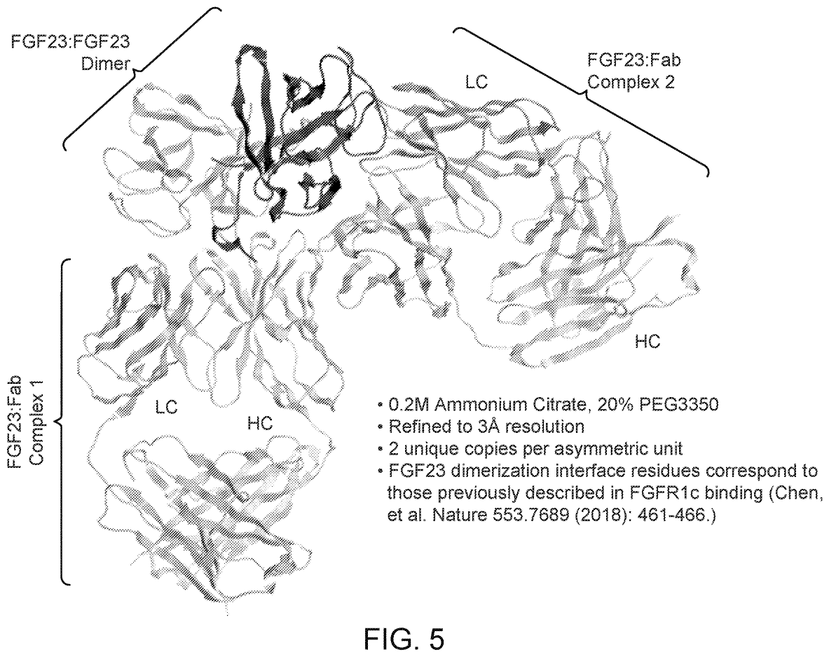

FIG. 5 shows the hFGF23(R179Q):burosumab Fab crystal structure with two antigen:Fab complexes interfaced by a FGF23:FGF23 dimer. It is noteworthy that FGF23 dimerization interface residues are not present in the epitopes identified in the crystal structure, but some are involved in FGFR1c receptor binding. It was further noted that burosumab binding to FGF23 overlaps with previously reported heparin sulfate binding residues (Goetz, Regina, et al. “Molecular insights into the klotho-dependent, endocrine mode of action of fibroblast growth factor 19 subfamily members.” Molecular and cellular biology 27.9 (2007): 3417-3428.). Heparin sulfate is known to be required for dimerization of the FGF23:aKlotho:FGFR1c complex (Chen. G., Liu, Y., Goetz, R. et al. “α-Klotho is a non-enzymatic molecular scaffold for FGF23 hormone signalling.” Nature 553, 461-466 (2018).). FGF23 dimerization interface residues in the present crystal structure correspond to those previously described in FGFR1c binding by Chen, et al. 2018. These findings suggest that burosumab binding to FGF23 may additionally compete with or otherwise interfere with receptor binding as well as dimerization.

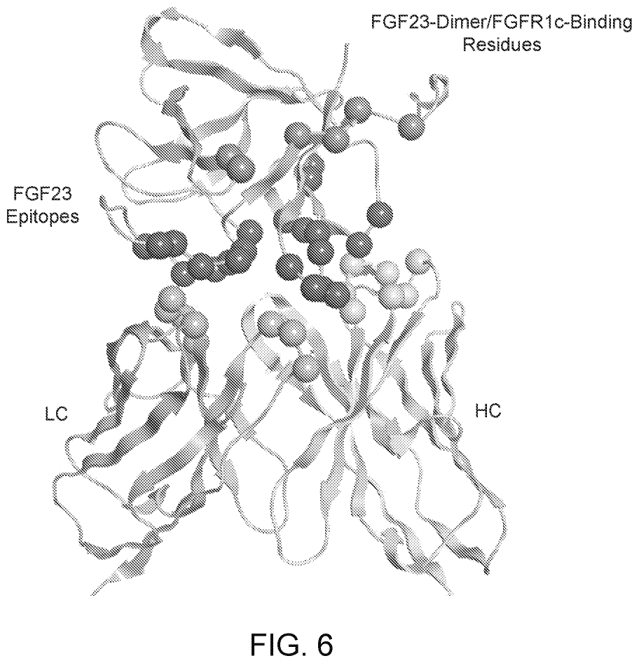

FIG. 6 is a ribbon model of the FGF23:Fab complex with FGF23 epitope residues identified as colored purple spheres and FGF23-dimer/FGFR1c-binding residues as colored cyan spheres, burosumab light chain paratope residues as colored green spheres and burosumab heavy chain paratope residues as colored yellow spheres. Identification of the epitope and paratope residues by crystallography enabled rational design of burosumab variant molecules described in Example 6 herein.

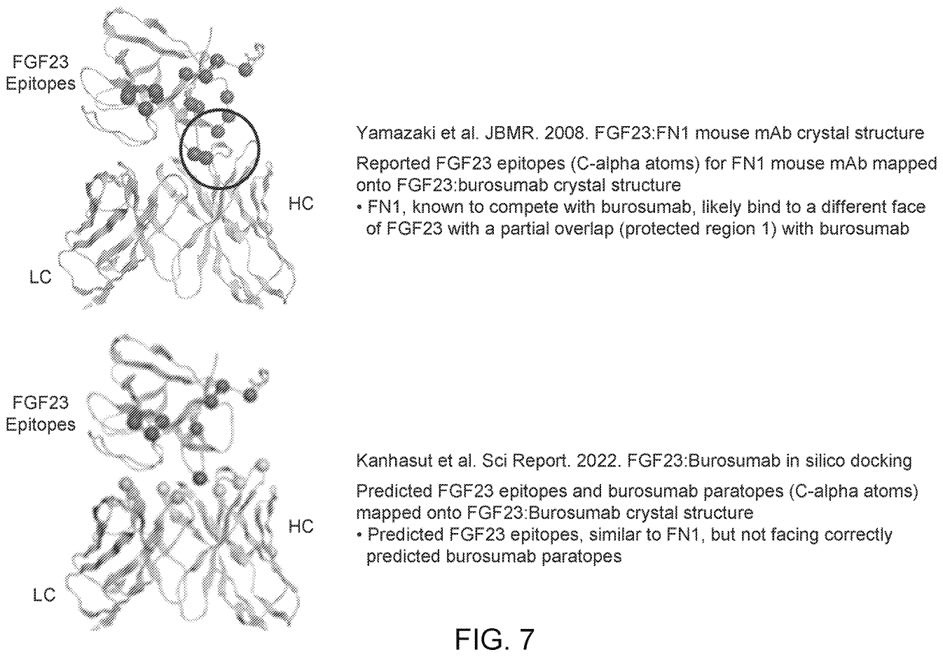

Overall, 3 Å resolution crystallographic FGF23:burosumab complex structure provides insight into the antigen:antibody interface, which is distinct from previously reported interfaces in related systems (e.g. FN1 antibody) or predicted interface (Kanhasut et al. 2022) (compare epitope residues—purple spheres—in FIG. 6 versus FIG. 7). Table 11 and Table 12 show the amino acid residues in human FGF23 (R179Q) and burosumab long chain and heavy chain sequences found to be involved in the structural interface of the antibody-antigen complex. In Table 11: “Type” refers to the nature of the predicted interaction at the subject amino acid position(s), “D” stands for distance-based interactions, “H” stands for hydrogen bond-based interactions, and “I” stands for ionic interactions; “Dist” refers to distance in angstrom; “Freq” refers to the number of atom-atom contacts associated with the pair (backbone and side-chain); and “Area” is provided in square Angstroms and refers to difference in surface area upon changing the indicated amino acid.

TABLE 11
Crystal structure - Ab:FGF23 interface contacts

| | | | | Energy | Dis- | | |
|---|---|---|---|---|---|---|---|
| | Ab | | | (kcal/ | tance | | Area |
| Type | Chain | Ab | FGF23 | mol) | (Å) | Freq | (Å²) |
| D | L | Asn92 | Thr46 | 1.14 | 4.02 | 6 | 13.7 |
| DH | L | Asn92 | Ala47 | −1.75 | 4.07 | 3 | 7.94 |
| DH | L | Asp93 | Ala47 | −0.11 | 3.98 | 4 | 22.81 |
| D | L | Asp93 | Arg48 | −0.73 | 3.79 | 8 | 18.04 |
| D | L | Tyr94 | Arg48 | 25.87 | 3.88 | 21 | 41.23 |
| D | L | Phe95 | Arg48 | −0.3 | 4.16 | 2 | 8.3 |
| DH | L | Asp99 | Asn49 | −1.27 | 4.02 | 10 | 14.17 |
| D | H | Val101 | Asn49 | −0.28 | 3.96 | 4 | 12.04 |
| DIH | H | Asp102 | Arg140 | −18.75 | 3.81 | 17 | 20.08 |
| D | H | Ala103 | Arg140 | 0.26 | 3.97 | 1 | 5.27 |
| D | H | Asp105 | Arg140 | 0.13 | 4.39 | 1 | 3.01 |
| D | L | Asp50 | Ala141 | 0.3 | 3.58 | 3 | 16.01 |
| DH | L | Ser53 | Ala141 | −0.42 | 3.74 | 3 | 12.38 |
| DH | L | Asp102 | Ala141 | −1.13 | 3.99 | 2 | 7.29 |
| D | L | Ser31 | Arg143 | 0.68 | 3.91 | 6 | 15.4 |
| DIH | L | Asp50 | Arg143 | −10.44 | 3.69 | 15 | 29.33 |
| DH | L | Ser52 | Arg143 | −1.18 | 3.9 | 5 | 26.38 |
| DH | L | Ser30 | Asn150 | −1.24 | 3.85 | 18 | 33.28 |
| D | L | Ser30 | Pro151 | −0.14 | 3.69 | 6 | 10.28 |
| D | L | Ser30 | Pro152 | −0.07 | 3.78 | 6 | 10.01 |
| D | L | Ser31 | Pro152 | −0.32 | 3.93 | 6 | 14.28 |
| D | L | Ala32 | Pro152 | 0.1 | 4.48 | 1 | 2.63 |
| DH | L | Asp50 | Pro152 | −0.56 | 3.77 | 2 | 7.6 |
| D | L | Ala32 | Pro153 | 0.47 | 4.13 | 2 | 11.57 |
| D | L | Asn92 | Pro153 | 1.02 | 4.04 | 5 | 23.2 |
| D | L | Asp50 | Tyr154 | −1.11 | 3.84 | 12 | 22.08 |
| D | H | Val101 | Tyr154 | −0.37 | 4.1 | 6 | 21.93 |
| D | H | Asp102 | Tyr154 | 1 | 3.98 | 11 | 18.53 |
| D | H | Tyr33 | Arg48 | −0.26 | 3.86 | 4 | 19.34 |
| D | H | Trp47 | Arg48 | −0.17 | 3.98 | 1 | 6.28 |
| D | H | Ile50 | Arg48 | −0.45 | 3.95 | 4 | 16.24 |
| DH | H | Ser59 | Arg48 | −0.95 | 3.93 | 5 | 21.48 |
| D | H | Tyr33 | Asn49 | 0.21 | 4.11 | 11 | 9.8 |
| D | H | Ile50 | Asn49 | −0.46 | 4.26 | 3 | 6.76 |
| D | L | Phe91 | Asn49 | −0.43 | 4.04 | 4 | 11.07 |
| D | L | Phe95 | Asn49 | −0.6 | 4 | 6 | 13.54 |
| DH | H | Tyr33 | Ser50 | −1.76 | 3.75 | 11 | 18.51 |
| DH | H | Asn31 | Tyr51 | −0.72 | 4.01 | 5 | 11.3 |
| D | H | His32 | Tyr51 | −0.22 | 4.12 | 6 | 6.11 |
| D | H | Tyr33 | Tyr51 | 0.44 | 4.01 | 17 | 20.11 |
| DH | H | Asp99 | Tyr51 | −1.91 | 3.8 | 12 | 17.81 |
| DA | H | Val101 | Tyr51 | −0.84 | 4.17 | 14 | 38.24 |
| D | H | Asn31 | Gln54 | 0.14 | 4.35 | 2 | 7.88 |
| D | H | Asn31 | His56 | 0.07 | 4.43 | 1 | 5.83 |
| DH | H | Asn31 | Asp62 | −2.72 | 3.79 | 3 | 18.27 |
| D | H | Asn31 | Gly63 | −0.04 | 4.08 | 5 | 15.47 |
| D | H | Thr30 | Pro65 | −0.51 | 3.87 | 4 | 9.73 |
| D | H | Asn31 | Pro65 | −0.09 | 3.96 | 6 | 8.27 |
| D | H | His32 | Pro65 | 0.26 | 4.29 | 2 | 1.88 |
| D | H | Tyr33 | Pro65 | 0.31 | 4.19 | 11 | 15.76 |
| DH | H | Asn52 | Pro65 | −1.87 | 3.95 | 9 | 26.25 |
| D | H | Ile54 | Pro65 | −0.66 | 4.27 | 5 | 8.04 |
| D | H | Ile54 | His66 | −0.48 | 4.04 | 6 | 21.23 |
| D | H | Val101 | Leu138 | −0.94 | 3.85 | 8 | 23.59 |
| D | H | Val101 | Gly139 | −0.11 | 4.11 | 4 | 5.8 |
| D | H | Asp102 | Gly139 | −0.45 | 4.03 | 7 | 10.59 |
| D | L | Leu46 | Arg140 | −0.32 | 4.3 | 2 | 5.34 |
| D | H | Tvr49 | Arg140 | 0.61 | 4.1 | 21 | 27.29 |
| DIH | L | Glu55 | Arg140 | −11.85 | 3.54 | 6 | 21.48 |
| D | H | Ile100 | Arg140 | −0.85 | 3.9 | 9 | 33.99 |
| D | L | Tyr49 | Ala141 | 0.85 | 4.09 | 5 | 9.53 |
| D | H | Ser31 | Tyr154 | −0.15 | 3.74 | 2 | 3.82 |
| D | H | Ala32 | Tyr154 | −0.25 | 4.1 | 8 | 13.34 |

TABLE 12
Top burosumab positions targeted for antibody variant design

| | Paratope | Type of Interaction |
|---|---|---|
| Burosumab | A.A. Residue | with FGF23 |
| Light Chain Variable Region | I29 | CAVITY |
| Light Chain Variable Region | S30 | DIRECT |
| Light Chain Variable Region | S31 | DIRECT |
| Light Chain Variable Region | A32 | DIRECT |
| Light Chain Variable Region | V34 | INDIRECT |
| Light Chain Variable Region | D50 | DIRECT |
| Light Chain Variable Region | A51 | DIRECT |
| Light Chain Variable Region | S52 | DIRECT |
| Light Chain Variable Region | S53 | DIRECT |
| Light Chain Variable Region | F91 | DIRECT |
| Light Chain Variable Region | N92 | DIRECT |
| Light Chain Variable Region | D93 | INDIRECT |
| Light Chain Variable Region | Y94 | DIRECT |
| Heavy Chain Variable | T30 | INDIRECT |
| Region | | |
| Heavy Chain Variable | N31 | DIRECT |
| Region | | |
| Heavy Chain Variable | N52 | DIRECT |
| Region | | |
| Heavy Chain Variable | I54 | DIRECT |
| Region | | |
| Heavy Chain Variable | S59 | DIRECT |
| Region | | |
| Heavy Chain Variable | I100 | DIRECT |
| Region | | |
| Heavy Chain Variable | V101 | DIRECT |
| Region | | |
| Heavy Chain Variable | A103 | DIRECT |
| Region | | |

As can be seen from Table 11 and Table 12, the amino acid residues found to interact between FGF23 and burosumab fall within the protected region 1 residues and protected region 3 residues identified by HDX-MS (Example 2 above), validating these regions as important mediators of the FGF23-burosumab interaction. However, no residues within protected region 2 were found to be involved in the interaction. With regard to the paratope residues, the interfacing residues within the burosumab heavy chain overlap with HDX-MS data describing heavy chain paratope region 1 and region 2, but no interacting residues from heavy chain paratope region 3 were observed (see Example 3 above). Similarly, the interfacing residues within the burosumab light chain overlap with HDX-MS data describing light chain paratope region 1 and region 2, but no interacting residues from light chain paratope region 3 were observed (see Example 3 above).

Based on the crystal structure, it was concluded that the HDX-MS data of Example 2 correctly identified protected regions of the burosumab-bound FGF23 molecule; however, the crystal structure revealed that protected region 2 does not interact with burosumab. Rather, this region of FGF23 is likely protected by conformational change of the FGF23 structure upon binding of burosumab (i.e., self-protection). Accordingly, in silico docking runs (Example 4) biased on the HDX-MS data led to selection of molecular poses that were not reflective of the actual structure elucidated by crystallography. Even so, the molecular docking studies of Example 4 generated models that were structurally very similar to the ultimate crystal structure (data not shown)—these models, though, were not considered initially because of the lack of involvement of protected region 2 residues.

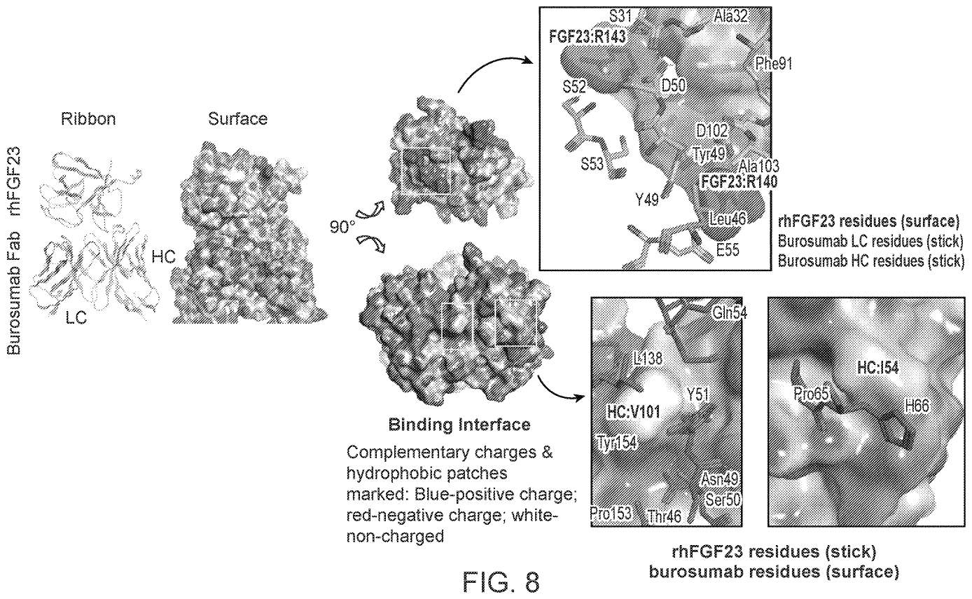

The crystal structure revealed complementary charges and hydrophobic patches involved in the burosumab-FGF23 interaction. FIG. 8 shows the FGF23:Fab complex in ribbon model (left) and surface models (right panels), with FGF23 and burosumab binding interfaces rotated into view and detailed amino acid positions superimposed as stick models (right-most panel). Positive-charged surfaces are shown in blue, negative charges in red, leaving non-charged surfaces in white. A positively charged region of FGF23 (R143 and R140) is seen to contact complementary negatively charged residues of burosumab and hydrophobic patches on the burosumab surface are seen to contact complementary non-charged, hydrophobic residues of FGF23 (L138, Y51, and H66) (see FIG. 8 insets detailing the binding interface).

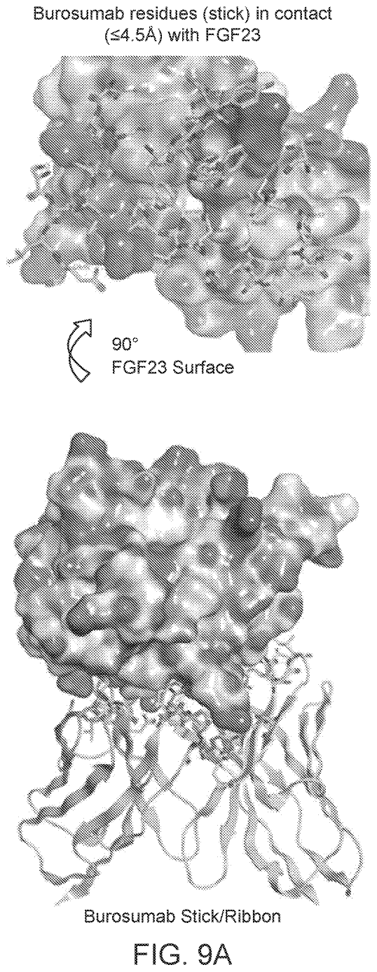

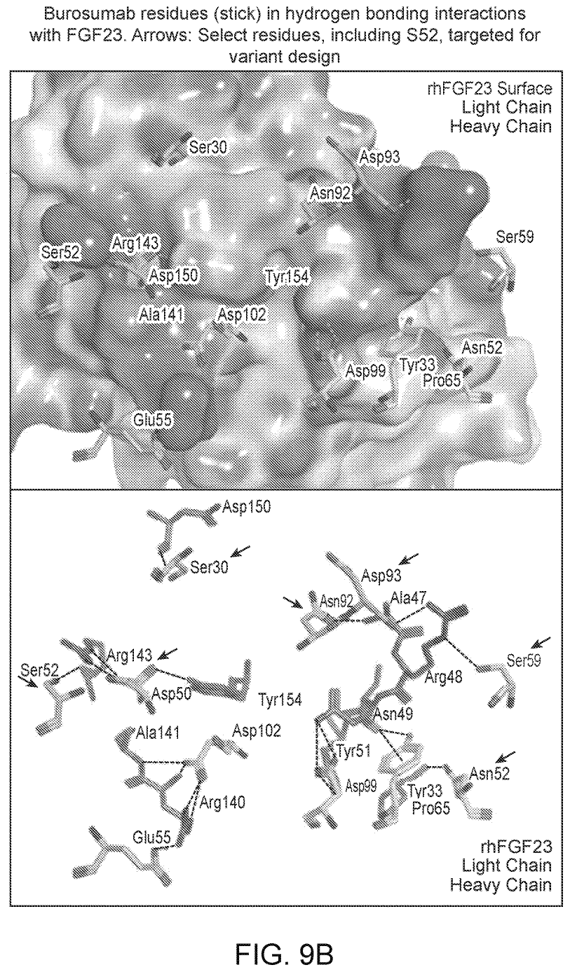

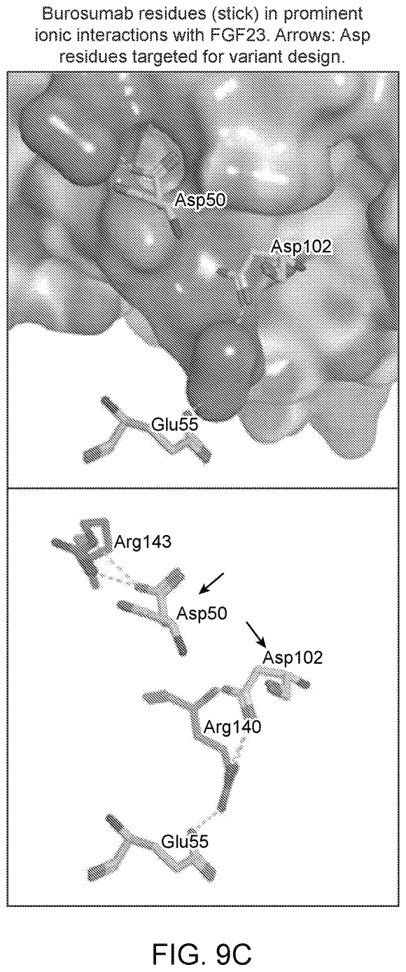

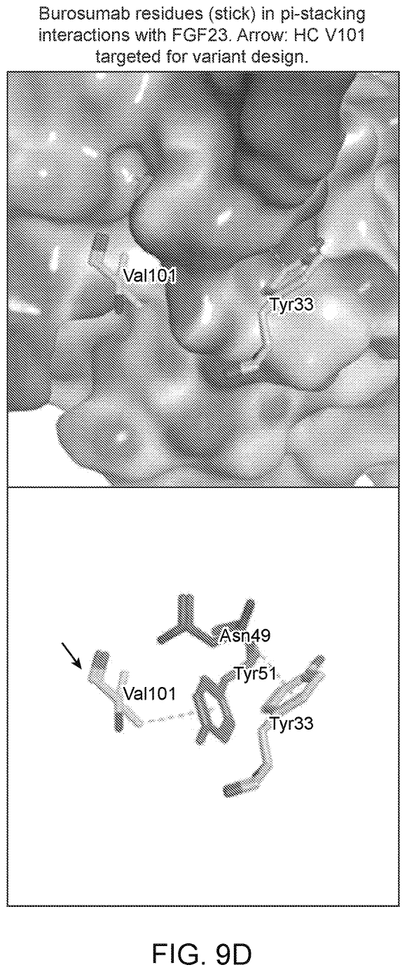

In particular, certain hydrogen-bonding residues were identified between burosumab and FGF23 which could be targeted to improve antibody binding affinity. FIG. 9 shows the FGF23 surface model interface with burosumab shown in ribbon and stick model. In surface models depicted, positive-charged surfaces are shown in blue, negative charges in red, leaving non-charged surfaces in white. Panel A shows the FGF23:Fab complex with rotation of the FGF23 portion 90 degrees to display the binding interface with all the burosumab residues in contact with FGF23 are shown. Panel B shows surface model of FGF23 (top) with burosumab hydrogen-bonding residue positions in stick model (superimposed and bottom). Panel C and Panel D show closer views of ionic interactions and aromatic stacking interactions at the FGF23:burosumab interface, respectively. In panel B, several select burosumab residues are marked (black arrows) as hydrogen-bond interaction sites which can be targeted for rational design across the interface with FGF23. Panel C focuses on prominent ionic interactions with burosumab aspartate and glutamate residues contacting arginine residues of FGF23 (R143 and R140). Panel D focuses on a pi-stacking interaction at FGF23 residue Y51 interacting with burosumab valine and tyrosine residues.

In sum, the combination of HDX-MS (Example 2 and Example 3), in silico modeling (Example 4), and crystallography data (present Example 7) enabled generation of a highly accurate and strongly predictive model of the FGF23:burosumab complex. All three lines of evidence were necessary to confidently identify burosumab residues that could be changed to alter the affinity of burosumab. Together, these findings elucidated burosumab residues likely to interface human FGF23 (see Table 12) which could serve as candidate residues for amino acid changes (deletions, additions, substitutions) for rational design of burosumab variants having improved affinity to FGF23.

### Example 8. Predictive Mutagenesis of Burosumab Based on Crystal Structure

A list of candidate amino acid changes to the variable heavy chain and variable light chain of burosumab was developed based on the crystal structure and interfacing contact residues described above in Example 7. Molecular mechanics-based tools were used to generate and optimize single (1×) and double (2×) amino acid changes focusing on 19 residue positions contacting the antigen and 10 residue positions semi-distal to antigen residues (i.e., 2nd shell distance of approximately 6-8 angstrom). In short, the crystal structure of the antibody in complex with Human FGF23, refined to a resolution of 3 Å, was utilized as basis to generate the starting coordinates upon which the molecular mechanics calculations were performed. The structure was assessed for completeness, gaps were filled with the corresponding residues and the termini were capped. The resulting structure was subjected to protonation (pH 7) and energy minimization as implemented in MOE 2022.02 (CCG) and structural geometry was checked prior to in silico mutagenesis.

The antibody-FGF23 interface was analyzed and relevant contacts were considered based on the distance between antibody and antigen residues. Residue positions on the antibody were selected and sorted into three groups: close contacts (3~4 Å), second shell contacts (up to 4.5 Å) and cavity/VdW interactions.

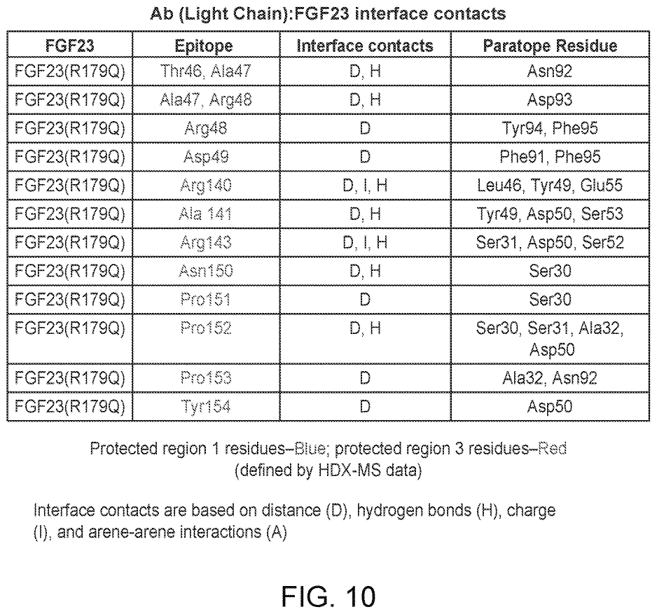

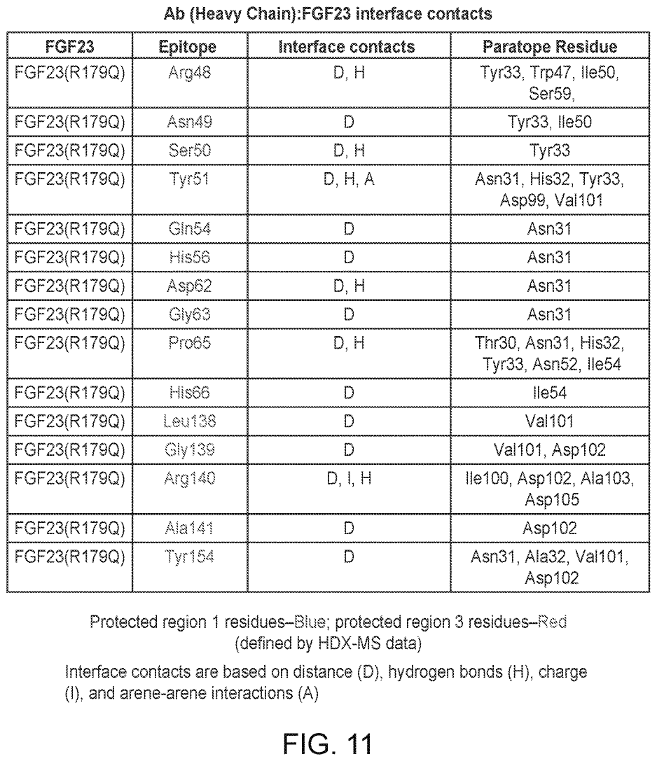

Three approaches were undertaken to improve affinity. First, variants were designed where a direct interaction was changed. Second, an indirect variant was designed to influence an already established direct interaction. And third, variants were designed that would fill an existing cavity. Emphasis was placed on hydrogen bonding interaction sites that could be modified by changing the interfacing burosumab residues to introduce ionic interactions or hydrophobic packing sites into aromatic pi-pi interactions, thereby increasing affinity. For example, burosumab light chain VL residue 552, which forms hydrogen-bond interactions with FGF23 Arg143, could be replaced with a negatively charged (D, E), large hydrogen-bonding (Q, T) or stacking (F, Y) that may strengthen binding/packing interactions. Similarly, a heavy chain residue Val101, which is stacking with FGF23 Tyr51, could be replaced with a bulkier residue (F, I, L, Y) or with a hydrogen-bonding property (T). Additional properties that were considered but not directly utilized for selections were: number of interactions per residue (connectivity), delta(SASA) and deltaG of interaction. FIG. 10 and FIG. 11 illustrate the relevant interface positions of the burosumab light chain and heavy chain, respectively, along with details of each residue's interactions with neighboring ones.

Each target position was initially scanned by 1× mutational screen with each mutation followed by rotameric optimization and refinement of the surrounding environment (4.5 Å). The resulting set of 420 variants was ranked based on energetics and structural properties and the top hits (i.e., ΔΔG<0 and dSASA<0) were combined into 2× and 3× variants for further screening and ranking. A final shortlist of 96 variants (1× and 2×) was generated based on structural analysis of each solution and utilized as basis to create a second list of expanded mutants to further explore the structural space surrounding the interface of interest.

The initial 1× scan using predictive mutagenesis resulted in 165 engineered FGF23-binding antibody designs, each comprising a single amino acid change relative to either the light chain variable region of burosumab (SEQ ID NO: 4) or the heavy chain variable region of burosumab (SEQ ID NO: 3). All mutational variants were evaluated and screened according to dAffinity (ΔΔG (kcal/mol)); dStability (ΔΔS (kcal/mol)) less than 3.0 kcal/mol; and dSASA (difference in solvent accessible surface area) as an indicator of gain or loss of buried surface upon introducing the mutation. ΔΔG is a metric for predicting how a single-point mutation (i.e., 1 amino acid change) will affect protein stability. It is a measure of the change in energy between the folded and unfolded states and the change in ΔG folding when a point mutation is present. This metric is used as a predictor of whether a point mutation will be favorable in terms of protein stability. The more negative the value, the more stable the protein. ΔΔG values>0.5 suggest that a mutation would be destabilizing; 0.5>ΔΔG>-0.5 values suggest changes should be considered neutral or near neutral; and ΔΔG<−0.5: suggest that the mutation would lead to a more stable protein. ΔΔS, on the other hand, is a measure of vibration entropy difference. A positive value indicates increased flexibility, while a negative value indicates more rigid protein. It is used for predicting how changing a given amino acid will change the entropy of the protein. Water accessible surface area (“VdW SA”) was also calculated. Table 13 provides a summary of the full 1× scan predictive mutagenesis results for burosumab light chain positions and Table 14 provides a summary of the full 1× scan predictive mutagenesis results for burosumab heavy chain positions.

TABLE 13
Burosumab VL 1X predictive mutagenesis results

| VL AA Change (relative | ΔΔG | ΔΔS | VdW SA |
|---|---|---|---|
| to SEQ ID NO: 4) | (kcal/mol) | (kcal/mol) | (Å²) |
| A25L | 0.04 | 0.76 | 21.9 |
| A25V | −0.04 | −0.26 | 13.3 |
| I29F | 1.061050981 | 1.632365369 | −28.147844 |
| I29L | −0.0728879 | 1.31929151 | −46.247868 |
| I29V | 0.267352586 | 1.703597137 | −16.464167 |
| S30D | −3.6718843 | 0.83782641 | −22.677042 |
| S30E | −2.1534008 | 0.50762112 | −29.107056 |
| S30F | −6.4906206 | −0.0122742 | −48.530941 |
| S30I | 2.286957776 | 0.549990944 | 22.1998 |
| S30L | −2.0238975 | 0.40896165 | −44.38591 |
| S30Q | −5.4928488 | 0.42036524 | −28.00009 |
| S30T | 0.648508526 | 0.928468736 | 21.659073 |
| S30V | −0.093856649 | 0.305082747 | 20.52256 |
| S30Y | −5.1055876 | 0.32176219 | −49.158344 |
| S31D | 3.56996088 | 0.63202685 | −26.274542 |
| S31E | 1.94516954 | 0.28280798 | −36.922615 |
| S31F | −2.5842167 | −0.7461013 | −54.371635 |
| S31I | 1.78221214 | −0.612691345 | 10.200069 |
| S31L | −0.155463589 | −0.263952687 | 11.979135 |
| S31Q | −1.7550465 | 0.55191751 | −18.653433 |
| S31T | −1.062041542 | 0.082099018 | 1.1834005 |
| S31V | 1.667246701 | −0.014243701 | 0.004099946 |
| S31Y | −2.7935664 | −0.467704 | −62.977528 |
| A32F | −1.09483682 | −0.359228605 | −21.611805 |
| A32I | 0.770987725 | −0.23905564 | −11.13908 |
| A32L | −1.67783134 | −0.656360123 | −9.6421356 |
| A32T | 0.502542557 | 0.410571808 | 0.085010208 |
| A32V | 1.83750345 | 0.406704427 | −0.54136229 |
| A32Y | −1.232772705 | −0.190198481 | −15.123551 |
| V34F | 1.39938938 | 2.11635117 | 2.813026 |
| V34I | −0.0978386 | 0.83360469 | −0.5620861 |
| V34L | −0.169944874 | 0.771558986 | −0.26748687 |
| V34Y | 0.483978126 | 2.777714634 | 8.7362003 |
| D50E | −0.39974246 | 0.935434385 | 20.788279 |
| D50F | 0.247562478 | 0.341023073 | 18.485962 |
| D50I | 2.422160839 | 0.06896623 | 8.5886898 |
| D50L | 4.759496344 | 0.345141119 | 47.131916 |
| D50Q | −3.307256369 | 0.471066083 | 0.15281084 |
| D50R | 5.222100064 | −0.239035501 | −20.571472 |
| D50S | −1.345785356 | 1.05077198 | 29.196266 |
| D50T | −2.802816003 | 0.633723865 | 34.229179 |
| D50V | −1.24099163 | 0.574804443 | 22.772923 |
| A51F | −0.032679132 | 1.824740267 | −3.2918215 |
| A51I | −0.341956382 | 0.844136509 | 1.9812078 |
| A51L | −0.435326019 | 0.219154085 | 0.025151623 |
| A51S | 0.590189424 | 1.108058478 | −5.2083364 |
| A51T | 0.541854453 | 0.65930264 | −4.8948936 |
| A51V | −0.874069958 | 0.174575554 | 1.8586986 |
| S52D | −0.4703946 | 1.30649565 | 9.6926641 |
| S52E | −0.3021981 | 1.31177809 | 11.95747 |
| S52F | −2.875389 | −0.3494975 | −10.131303 |
| S52Q | −0.143493129 | 0.884475034 | 25.364239 |
| S52T | 0.128606217 | 0.530664437 | 1.207417 |
| S52Y | −3.133793831 | −0.150231438 | 57.616718 |
| S53D | −4.9779845 | 0.37701257 | 12.876547 |
| S53E | −1.5124636 | 0.85673251 | 12.985263 |
| S53F | −4.4610498 | 0.13567548 | −2.0610542 |
| S53I | −2.613199991 | −1.08626583 | 0.62171143 |
| S53L | −1.16483418 | −0.242817586 | 24.409243 |
| S53Q | −2.034307873 | 0.57250637 | 34.919781 |
| S53R | −0.9929227 | 0.2709468 | −4.0244999 |
| S53T | −0.237225711 | 0.282621034 | 3.6471076 |
| S53V | −2.33446696 | −0.812313395 | −3.5696123 |
| S53Y | −5.0495748 | −0.0684582 | −6.7887955 |
| Q90F | −2.1 | −0.03 | −9.8 |
| Q90Y | −2.47 | −0.07 | −13.3 |
| F91K | −2.02 | 1.46 | −1.37 |
| F91R | −2.5 | 0.69 | −3.24 |
| F91Y | 0.073 | 1.13 | −1.4 |
| N92D | −1.1543339 | 0.85687799 | 6.6619544 |
| N92E | 0.655305 | 0.92451717 | −17.099483 |
| N92F | 0.14072043 | −0.2581115 | −6.1453514 |
| N92Q | 0.846794726 | 0.397366739 | 2.1917546 |
| N92S | 2.78115842 | 0.88155805 | −3.7792108 |
| N92T | 4.790090325 | 0.834014183 | −28.341656 |
| N92Y | −0.4386895 | −0.121354 | 4.7035565 |
| D93E | −0.17027592 | 0.595523751 | 3.0977747 |
| D93F | 2.31451482 | −0.969308461 | −36.287567 |
| D93Q | −0.92803509 | 0.676351664 | 4.1740527 |
| D93R | 1.31 | 0.24 | 9.98 |
| D93S | 1.675917835 | 0.389550331 | −27.250326 |
| D93T | 3.624540183 | −0.031258601 | 0.62384415 |
| D93Y | 0.167244776 | −0.72587811 | −0.26277241 |
| Y94D | 1.87364442 | 1.00868027 | −18.932884 |
| Y94E | 2.30058187 | 1.24293306 | −38.1619 |
| Y94F | 0.26675234 | 0.54350757 | −12.910084 |
| Y94Q | 2.060892689 | 0.66556261 | −48.488823 |
| Y94S | 2.79793208 | 1.1990931 | −19.928265 |
| Y94T | 2.088321013 | 0.91544587 | −44.222107 |

TABLE 14
Burosumab VH 1X predictive mutagenesis results

| VH AA Change (relative | ΔΔG | ΔΔS | VdW SA |
|---|---|---|---|
| to SEQ ID NO: 3) | (kcal/mol) | (kcal/mol) | (Å²) |
| T30E | 0.684137201 | 1.075241818 | 18.717197 |
| T30F | 0.18770538 | 0.83180798 | 15.709345 |
| T30Q | 0.329924618 | 0.977560421 | 18.959789 |
| T30S | 0.4350999 | 1.028327716 | −1.8230846 |
| T30Y | 0.181118285 | 0.677831651 | 55.050991 |
| N31D | 2.965937522 | 0.843428248 | 8.5089788 |
| N31E | 3.141789251 | 0.790633166 | 4.7221541 |
| N31F | 1.505071443 | 0.156433609 | −19.040478 |
| N31Q | −0.658144392 | 0.474920713 | −5.7396598 |
| N31R | −0.505348545 | −0.091904039 | −18.215101 |
| N31Y | −1.320720057 | 0.171386205 | 76.718956 |
| H32E | 0.531400637 | 1.365501752 | −37.351734 |
| H32F | −0.01862203 | −0.408667274 | −31.556114 |
| H32Q | −1.634162952 | 0.91749504 | −27.375647 |
| H32S | 0.200916565 | 1.429533882 | 14.526258 |
| H32T | 0.082050769 | 0.718451694 | −35.913139 |
| I50D | −3.728931574 | 3.567036759 | 19.925985 |
| I50E | −2.785319845 | 2.79166681 | 0.059334006 |
| I50F | −7.869489552 | 0.861678876 | −11.075091 |
| I50S | −3.535387748 | 3.347888483 | 19.529911 |
| I50T | −0.360198315 | 1.515972972 | 9.489028 |
| I50Y | −2.70969497 | 1.614834522 | −11.143722 |
| N52E | 0.51388448 | 0.79113671 | 38.100883 |
| N52F | 5.20131785 | 0.51759159 | −9.5689497 |
| N52Q | 1.635186438 | 0.928549811 | 19.386501 |
| N52Y | 4.89901696 | 0.84897734 | −7.5632229 |
| I54E | 4.723370263 | 2.798599057 | −13.66046 |
| I54F | 0.925662374 | 1.594214175 | 46.908218 |
| I54L | 2.609985936 | 1.638635049 | 6.9898171 |
| I54Q | 2.457363728 | 2.490175263 | 18.760778 |
| I54S | 2.674823664 | 2.12181984 | −26.007236 |
| I54T | 3.80950452 | 2.246885328 | −4.9698415 |
| I54V | 2.939266531 | 1.455407636 | −6.6988673 |
| I54Y | 0.214285677 | 1.82058734 | 41.796417 |
| S55E | 0.420372588 | 0.92585963 | 29.211838 |
| S55F | −1.053741554 | 0.889186473 | 90.757767 |
| S55Q | −2.385053801 | 0.153300959 | 42.401405 |
| S55T | −0.458709886 | 0.334143886 | 18.080853 |
| S55Y | −0.395620536 | 0.712916488 | 95.909492 |
| S59D | 0.86873275 | 0.93661783 | 11.031759 |
| S59E | 1.42930367 | 0.99314984 | −0.1969754 |
| S59F | −2.4419416 | −0.214348 | −3.5550513 |
| S59I | −3.9262363 | 0.07231091 | 3.0604253 |
| S59L | −5.0521902 | −0.0593502 | 11.594638 |
| S59Q | −4.7749639 | 0.34293134 | −18.870623 |
| S59T | −4.596499 | 0.41333773 | 6.0870099 |
| S59V | −4.7103284 | 0.03496928 | 4.5306883 |
| S59Y | −1.6484989 | 0.52900749 | 47.218006 |
| D99E | −0.232029036 | 0.837742101 | −10.336287 |
| D99Q | −0.515419631 | 1.354701357 | −4.7501192 |
| D99Y | 2.55 | −0.07 | −3.24 |
| I100D | 3.863436116 | 3.059680528 | 14.038824 |
| I100E | 1.24916155 | 3.7849893 | −6.1821141 |
| I100F | 1.413449602 | 1.211012567 | −3.7273011 |
| I100L | 3.025129226 | 1.291439304 | 11.713667 |
| I100Q | 1.457092754 | 2.276656577 | 30.016344 |
| I100V | 4.501027071 | 1.853071862 | 37.992546 |
| I100Y | 1.196847279 | 1.623909144 | 50.271866 |
| V101F | 2.3716018 | 0.65795356 | −68.693504 |
| V101I | −1.9797864 | 0.2883803 | −12.589581 |
| V101L | −0.6986304 | 0.70348261 | −6.3387203 |
| V101T | 0.332725095 | 1.418010059 | −3.6793778 |
| V101Y | 0.70072691 | 0.683377056 | −19.356924 |
| D102E | 7.977327323 | 0.609388647 | −7.7275596 |
| D102F | 5.034696184 | −0.615590492 | −32.069496 |
| D102I | 3.502175813 | −0.214283484 | −16.338041 |
| D102L | 0.032551406 | −0.396340168 | −19.263794 |
| D102Q | 9.600856797 | 1.604072636 | −6.4905972 |
| D102Y | 8.24642893 | −0.280662341 | −24.580505 |
| A103D | −2.96021076 | 2.462046405 | −5.0743966 |
| A103E | −3.769332308 | 2.929902207 | 13.589715 |
| A103F | −2.123937787 | 0.593947072 | −36.6259 |
| A103Q | −7.284866478 | 1.054712643 | 16.935923 |
| A103S | −3.532337047 | 0.779836492 | −1.1532063 |
| A103T | 0.030754857 | 0.821138114 | 2.9620366 |
| A103Y | −3.31726254 | 0.09784497 | −5.0800767 |

The 2× scan using predictive mutagenesis for double amino acid changes relative to burosumab heavy and light chain variable regions resulted in an additional 20 engineered FGF23-binding antibody designs, each comprising two amino acid changes relative to the light chain variable region of burosumab (SEQ ID NO: 4) and/or the heavy chain variable region of burosumab (SEQ ID NO: 3). Table 15 provides a summary of the 2× scan predictive mutagenesis results.

TABLE 15
Burosumab 2X Scan predictive mutagenesis results

| AA | AA | ΔΔg | ΔΔS | VdW |
|---|---|---|---|---|
| Change 1 | Change 2 | (kcal/mol) | (kcal/mol) | SA (Å²) |
| VL: S31E | VL: D50F | −4.9818861 | −0.1303292 | 5.2358189 |
| VH: N31F | VH: S59F | −4.1762399 | −1.080179 | −17.86721 |
| VL: D50F | VL: S53E | −3.5323026 | 0.23339506 | 21.309801 |
| VL: S31F | VL: S52E | −3.3569123 | −0.3131565 | −29.340513 |
| VL: D50F | VH: V101I | −2.6177099 | 0.20896309 | −0.251417 |
| VH: S59D | VH: V101I | −2.3354685 | 0.43520707 | −18.403811 |
| VL: S31F | VL: S53E | −2.2152397 | −0.0631116 | −26.779749 |
| VL: N92E | VL: D93F | −1.372372 | −0.2463043 | −26.993782 |
| VL: S31E | VL: S53E | −0.366416 | 0.61385435 | −2.1146641 |
| VL: S31F | VL: D50F | −0.3102773 | −1.383143 | −38.363171 |
| VH: N31E | VH: S59E | 0.46434414 | 1.54769119 | 11.696677 |
| VH: S57E | VH: S59D | 0.95383059 | 1.34642839 | 33.086407 |
| VH: N31E | VH: I54F | 1.17787603 | 1.53083549 | 23.253418 |
| VH: S59T | VH: V101L | 1.93695757 | 0.72477322 | −18.162439 |
| VH: I54Y | VH: V101F | 2.69242757 | 1.60681199 | −29.235994 |
| VH: N52F | VH: V101I | 2.94986019 | 0.00652214 | −24.637529 |
| VL: N92D | VL: D93F | 2.9614789 | −0.573638 | −43.874622 |
| VH: N52F | VH: V101L | 3.54531105 | 0.55270088 | −54.158905 |
| VL: S31F | VL: D50E | — | — | — |
| VL: Y94D | VH: S59D | — | — | — |

### Example 9. Engineered FGF23-Binding Antibody Design Panel 2 Based on Crystal Structure

Table 16 shows 76 engineered FGF23-binding antibody designs from the 1× scan per Examples 7 and 8. Each variant is labeled with a sequential Engineered FGF23-binding Antibody Structure ID numbered from 101 through 176 and has one amino acid change relative to the parental burosumab heavy chain variable region amino acid sequence (SEQ ID NO: 3) or the parental burosumab light chain variable region amino acid sequence (SEQ ID NO: 4). For clarity, each antibody represented by Antibody Structure ID numbers UGX101 through UGX176 has the parental burosumab heavy chain sequence (SEQ ID NO: 1) and light chain sequence (SEQ ID NO: 2) save for the indicated amino acid change relative to those sequences.

Table 17 shows 20 engineered FGF23-binding antibody designs incorporating two amino acid changes (2× scan) per Examples 7 and 8. Each variant is labeled with a sequential Engineered FGF23-binding Antibody Structure ID numbered from 177 through 196 and has two amino acid changes relative to the parental burosumab heavy chain variable region amino acid sequence (SEQ ID NO: 3) and/or the parental burosumab light chain variable region amino acid sequence (SEQ ID NO: 4). For clarity, each antibody represented by Antibody Structure ID numbers UGX177 through UGX196 has the parental burosumab heavy chain sequence (SEQ ID NO: 1) and light chain sequence (SEQ ID NO: 2) save for the indicated amino acid changes relative to those sequences.

TABLE 16
1X Design panel 2 of Engineered FGF23-binding antibodies

| Engineered | VL AA Change | Engineered | VH AA Change |
|---|---|---|---|
| FGF23-Binding | (relative | FGF23-Binding | (relative |
| Antibody | to SEQ | Antibody | to SEQ |
| Structure ID | ID NO: 4) | Structure ID | ID NO: 3) |
| UGX101 | I29F | UGX148 | T30F |
| UGX102 | I29L | UGX149 | N31D |
| UGX103 | S30D | UGX150 | N31E |
| UGX104 | S30E | UGX151 | N31F |
| UGX105 | S30F | UGX152 | N31Q |
| UGX106 | S30L | UGX153 | N31R |
| UGX107 | S30Q | UGX154 | N52F |
| UGX108 | S30Y | UGX155 | N52Y |
| UGX109 | S31D | UGX156 | I54F |
| UGX110 | S31E | UGX157 | I54Y |
| UGX111 | S31F | UGX158 | S59D |
| UGX112 | S31Q | UGX159 | S59E |
| UGX113 | S31Y | UGX160 | S59F |
| UGX114 | A32F | UGX161 | S59I |
| UGX115 | A32V | UGX162 | S59L |
| UGX116 | V34F | UGX163 | S59Q |
| UGX117 | V34I | UGX164 | S59T |
| UGX118 | D50F | UGX165 | S59V |
| UGX119 | D50L | UGX166 | S59Y |
| UGX120 | D50Q | UGX167 | I100D |
| UGX121 | D50R | UGX168 | I100E |
| UGX122 | D50S | UGX169 | I100F |
| UGX123 | D50T | UGX170 | V101F |
| UGX124 | D50V | UGX171 | V101I |
| UGX125 | A51V | UGX172 | V101L |
| UGX126 | S52D | UGX173 | A103D |
| UGX127 | S52E | UGX174 | A103F |
| UGX128 | S52F | UGX175 | A103S |
| UGX129 | S53D | UGX176 | A103T |
| UGX130 | S53E | | |
| UGX131 | S53F | | |
| UGX132 | S53R | | |
| UGX133 | S53Y | | |
| UGX134 | F91Y | | |
| UGX135 | N92D | | |
| UGX136 | N92E | | |
| UGX137 | N92F | | |
| UGX138 | N92S | | |
| UGX139 | N92Y | | |
| UGX140 | D93F | | |
| UGX141 | D93Q | | |
| UGX142 | D93S | | |
| UGX143 | D93T | | |
| UGX144 | Y94D | | |
| UGX145 | Y94E | | |
| UGX146 | Y94F | | |
| UGX147 | Y94S | | |

TABLE 17
2X Design panel 2 of Engineered FGF23-binding antibodies

| Engineered FGF23- | VL AA Change | VH AA Change |
|---|---|---|
| Binding Antibody | (relative to | (relative to |
| Structure ID | SEQ ID NO: 4) | SEQ ID NO: 3) |
| UGX177 | S31E, D50F | |
| UGX178 | | N31F, S59F |
| UGX179 | D50F, S53E | |
| UGX180 | S31F, S52E | |
| UGX181 | D50F | V101I |
| UGX182 | | S59D, V101I |
| UGX183 | S31F, S53E | |
| UGX184 | N92E, D93F | |
| UGX185 | S31E, S53E | |
| UGX186 | S31F, D50F | |
| UGX187 | | N31E, S59E |
| UGX188 | | S57E, S59D |
| UGX189 | | N31E, I54F |
| UGX190 | | S59T, V101L |
| UGX191 | | I54Y, V101F |
| UGX192 | | N52F, V101I |
| UGX193 | N92D, D93F | |
| UGX194 | | N52F, V101L |
| UGX195 | S31F, D50E | |
| UGX196 | Y94D | S59D |

### Example 10. Tn Vitro Testing of Select Variants for Increased Affinity for FGF23

FGF23-binding antibody designs from the 1× and 2× antibody design panels shown in Tables 16 and 17, respectively, along with additional antibody variants having 1 or 2 amino acid changes relative to burosumab were evaluated for affinity by surface plasmon resonance (SPR).

96 antibody variants were expressed at small scale (3 ml) and screened by SPR analysis against human FGF23 (R179Q) at a single concentration (100 nM). Experimental setup of SPR analysis: SPR device—8K+; chip preparation—CM5 hIgG chip; channels—1-8; immobilization concentration—1.5 ug/ml; immobilization method—capture; flow rate—5 μl/min; contact time—60 s; running buffer—1×HBS-EP+pH 7.4; Temperature—20° C.; flow rate—50 μl/min; association time—270 s; dissociation time—600 s; antigen—rhFGF23(R179Q); top concentration—0.1 μM.

All antibody variants were ranked by off rate k_{d} or equilibrium dissociation constant K_{D}. Top hits with best improvement in affinity relative to un-modified burosumab were confirmed with concentration response curves (CRC). Experimental setup of SPR analysis for CRC: SPR device—8K+; chip preparation—CM5 hIgG chip; channels—1-8; immobilization concentration—1.5 ug/ml; immobilization method—capture: flow rate—5 μl/min: contact time—60 s; running buffer—1×HBS-EP+pH 7.4; temperature—20° C.; flow rate—50 μl/min; association time—120 s; dissociation time—1800 s; antigen—rhFGF23(R179Q); top concentration—0.1 μM, 9-pt 3-fold Single Cycle Kinetics.

Results from SPR screening and CRC evaluation are provided in Table 18, which shows K_{D} and determination of final kinetic parameters kₐ and k_{d} as well as surface activity for select engineered antibody variants. In Table 18, column headers are abbreviated as follows: “AS ID” refers to “Antibody Structure ID”, a unique identifier for each antibody as defined herein: “AA Change(s)” refers to amino acid residue changes relative to either the parental burosumab heavy chain (“HC”) variable region amino acid sequence (SEQ ID NO: 3) or the parental burosumab light chain (“LC”) variable region amino acid sequence (SEQ ID NO: 4), or both; “CL” refers to capture level (RU): “K_{D}” refers to the equilibrium dissociation constant (M); “kₐ” refers to the association rate or constant (1/Ms): “kₐ” refers to dissociation rate or constant (1/s); “Rₘₐₓ” refers to theoretical Rₘₐₓ or calculated maximum observable signal (relative units): “SA” refers to surface activity (%); and “Ratio” refers to the calculated ratio of the variant antibody K_{D}/burosumab K_{D}.

TABLE 18
In vitro testing of select antibody variants by SPR

| AS ID | AA Change(s) | CL | K_{D} | Kₐ | k_{d} | RU | SA | Ratio |
|---|---|---|---|---|---|---|---|---|
| UGX205 | HC:S59E, LC:Y94D | 152.7 | 4.07E−12 | 1.53E+07 | 6.25E−05 | 59.2 | 93.9 | 5 |
| UGX201 | HC:S59E, LC:N92F | 79.6 | 5.17E−12 | 1.12E+07 | 5.81E−05 | 30.9 | 83.4 | 4 |
| UGX144 | LC:Y94D | 168.5 | 5.77E−12 | 1.09E+07 | 6.31E−05 | 65.4 | 89.5 | 4 |
| UGX157 | HC:I54Y | 184.1 | 7.58E−12 | 1.49E+07 | 1.13E−04 | 71.4 | 89.1 | 3 |
| UGX202 | HC:S59E, LC:N92Y | 146.3 | 7.59E−12 | 1.29E+07 | 9.80E−05 | 56.7 | 91.6 | 3 |
| UGX175 | HC:A103S | 188.5 | 7.72E−12 | 1.24E+07 | 9.61E−05 | 73.1 | 86.8 | 3 |
| UGX206 | HC:S59E, LC:Y94E | 51.8 | 7.87E−12 | 6.25E+06 | 4.92E−05 | 20.1 | 107.6 | 3 |
| UGX202 | HC:S59E, LC:N92Y | 131.9 | 8.07E−12 | 1.17E+07 | 9.45E−05 | 51.1 | 91.5 | 3 |
| UGX182 | HC:S59D, HC:V101I | 94.2 | 9.47E−12 | 1.49E+07 | 1.41E−04 | 36.5 | 100.5 | 2 |
| UGX156 | HC:I54F | 111.5 | 1.05E−11 | 5.94E+06 | 6.25E−05 | 43.2 | 105.9 | 2 |
| UGX206 | HC:S59E, LC:Y94E | 55.9 | 1.09E−11 | 7.69E+06 | 8.37E−05 | 21.7 | 85.9 | 2 |
| UGX203 | HC:S59E, LC:S52D | 118.5 | 1.15E−11 | 1.26E+07 | 1.45E−04 | 45.9 | 81.4 | 2 |
| UGX126 | LC:S52D | 145.0 | 1.24E−11 | 1.05E+07 | 1.30E−04 | 56.2 | 86.5 | 2 |
| UGX145 | LC:Y94E | 120.6 | 1.33E−11 | 6.42E+06 | 8.52E−05 | 46.8 | 87.4 | 2 |
| UGX204 | HC:S59E, LC:S52E | 120.2 | 1.33E−11 | 1.40E+07 | 1.86E−04 | 46.6 | 90.4 | 2 |
| UGX160 | HC:S59F | 155.2 | 1.42E−11 | 6.15E+06 | 8.70E−05 | 60.2 | 94.5 | 1 |
| UGX160 | HC:S59F | 143.0 | 1.47E−11 | 6.53E+06 | 9.61E−05 | 55.4 | 92.6 | 1 |
| UGX139 | LC:N92Y | 137.0 | 1.64E−11 | 6.32E+06 | 1.04E−04 | 53.1 | 86.2 | 1 |
| UGX127 | LC:S52E | 90.0 | 1.76E−11 | 6.93E+06 | 1.22E−04 | 34.9 | 85.0 | 1 |
| UGX188 | HC:S57E, HC:S59D | 158.7 | 1.83E−11 | 1.39E+07 | 2.53E−04 | 61.5 | 95.8 | 1 |
| UGX178 | HC:N31F, HC:S59F | 159.4 | 2.12E−11 | 6.33E+06 | 1.34E−04 | 61.8 | 85.6 | 1 |
| UGX166 | HC:S59Y | 176.8 | 2.24E−11 | 8.77E+06 | 1.96E−04 | 68.5 | 93.1 | 1 |
| UGX159 | HC:S59E | 212.3 | 2.26E−11 | 9.91E+06 | 2.24E−04 | 82.3 | 93.0 | 1 |
| UGX135 | LC:N92D | 101.3 | 2.35E−11 | 7.71E+06 | 1.81E−04 | 39.3 | 100.4 | 1 |
| UGX140 | LC:D93F | 135.0 | 2.42E−11 | 2.63E+06 | 6.37E−05 | 52.3 | 103.1 | 1 |
| UGX159 | HC:S59E | 163.3 | 2.51E−11 | 8.83E+06 | 2.22E−04 | 63.4 | 97.9 | 1 |
| UGX151 | HC:N31F | 193.3 | 2.55E−11 | 8.32E+06 | 2.12E−04 | 74.9 | 90.3 | 1 |
| UGX148 | HC:T30F | 157.8 | 2.72E−11 | 6.65E+06 | 1.81E−04 | 61.1 | 91.2 | 1 |
| UGX137 | LC:N92F | 137.2 | 3.19E−11 | 5.04E+06 | 1.61E−04 | 53.2 | 86.5 | 1 |
| UGX146 | LC:Y94F | 91.1 | 3.32E−11 | 5.14E+06 | 1.70E−04 | 35.3 | 89.9 | 1 |
| UGX164 | HC:S59T | 201.0 | 3.36E−11 | 5.64E+06 | 1.90E−04 | 77.9 | 91.9 | 1 |
| UGX130 | LC:S53E | 169.1 | 3.48E−11 | 1.03E+07 | 3.58E−04 | 65.5 | 88.4 | 1 |
| UGX165 | HC:S59V | 159.4 | 3.63E−11 | 4.82E+06 | 1.75E−04 | 61.9 | 95.4 | 1 |
| UGX131 | LC:S53F | 82.5 | 3.93E−11 | 6.78E+06 | 2.66E−04 | 31.9 | 95.7 | 1 |
| UGX152 | HC:N31Q | 97.2 | 4.16E−11 | 6.29E+06 | 2.62E−04 | 37.7 | 91.2 | 0 |
| UGX165 | HC:S59V | 166.7 | 4.48E−11 | 4.92E+06 | 2.20E−04 | 64.6 | 90.9 | 0 |
| UGX117 | LC:V34I | 120.5 | 5.04E−11 | 3.35E+06 | 1.69E−04 | 46.7 | 86.9 | 0 |
| UGX161 | HC:S59I | 236.6 | 6.10E−11 | 4.38E+06 | 2.67E−04 | 91.7 | 84.2 | 0 |
| UGX117 | LC:V34I | 152.5 | 7.03E−11 | 2.90E+06 | 2.04E−04 | 59.1 | 94.6 | 0 |
| UGX162 | HC:S59L | 174.5 | 9.86E−11 | 4.79E+06 | 4.72E−04 | 67.6 | 88.4 | 0 |
| UGX153 | HC:N31R | 422.9 | 1.12E−10 | 2.87E+06 | 3.22E−04 | 164.0 | 83.7 | 0 |
| UGX133 | LC:S53Y | 21.2 | 2.02E−10 | 3.63E+06 | 7.33E−04 | 8.2 | 84.8 | 0 |
| UGX190 | HC:S59T, HC:V101L | 160.4 | 3.08E−09 | 3.35E+06 | 1.03E−02 | 62.2 | 92.7 | 0 |
| UGX192 | HC:N52F, HC:V101I | 166.4 | 2.06E−08 | 1.15E+06 | 2.36E−02 | 64.5 | 82.3 | 0 |
| UGX201 | HC:S59E, LC:N92F | 38.6 | 4.52E−12 | 8.08E+06 | 3.65E−05 | 14.9 | 99.0 | 5 |
| UGX201 | HC:S59E, LC:N92F | 76.7 | 6.32E−12 | 9.99E+06 | 6.31E−05 | 29.7 | 85.3 | 3 |
| UGX144 | LC:Y94D | 175.3 | 6.58E−12 | 1.07E+07 | 7.03E−05 | 68.0 | 89.8 | 3 |
| UGX206 | HC:S59E, LC:Y94E | 91.1 | 6.62E−12 | 8.64E+06 | 5.72E−05 | 35.3 | 84.0 | 3 |
| UGX202 | HC:S59E, LC:N92Y | 121.5 | 7.53E−12 | 1.18E+07 | 8.91E−05 | 47.1 | 91.7 | 3 |
| UGX156 | HC:I54F | 196.5 | 9.24E−12 | 1.06E+07 | 9.77E−05 | 76.2 | 90.8 | 2 |
| UGX203 | HC:S59E, LC:S52D | 102.3 | 1.13E−11 | 1.28E+07 | 1.45E−04 | 39.7 | 83.5 | 2 |
| UGX203 | HC:S59E, LC:S52D | 61.5 | 1.24E−11 | 1.08E+07 | 1.34E−04 | 23.8 | 85.3 | 2 |
| UGX145 | LC:Y94E | 129.2 | 1.38E−11 | 7.07E+06 | 9.74E−05 | 50.1 | 86.4 | 1 |
| UGX204 | HC:S59E, LC:S52E | 107.9 | 1.40E−11 | 8.64E+06 | 1.21E−04 | 41.8 | 94.6 | 1 |
| UGX160 | HC:S59F | 129.9 | 1.46E−11 | 6.16E+06 | 9.01E−05 | 50.4 | 93.2 | 1 |
| UGX204 | HC:S59E, LC:S52E | 105.0 | 1.83E−11 | 7.57E+06 | 1.38E−04 | 40.7 | 95.3 | 1 |
| UGX140 | LC:D93F | 135.8 | 1.06E−10 | 2.27E+06 | 2.42E−04 | 52.6 | 74.0 | 0 |

These data show that improved affinity in engineered burosumab variants is most likely due to improved dissociation rate (off-rate, or k_{d}), whereas the association rate (on-rate, or kₐ) remained very similar across all variants. These data also demonstrate at least 13 distinct engineered burosumab variants having a K_{D} ratio of greater than or equal to 2, meaning that these antibody variants have at least a 2-fold improvement (up to a 5-fold improvement) in affinity compared to burosumab. Antibodies showing slower or similar off-rates compared to burosumab were selected for further analysis using concentration response curves (CRCs) to confirm kinetic parameters.

Results are provided in Table 19, ranked according to off-rate. In Table 19, column headers are abbreviated as follows: “AS ID” refers to “Antibody Structure ID”, a unique identifier for each antibody as defined herein; “AA Change(s)” refers to amino acid residue changes relative to either the parental burosumab heavy chain (“HC”) variable region amino acid sequence (SEQ ID NO: 3) or the parental burosumab light chain (“LC”) variable region amino acid sequence (SEQ ID NO: 4), or both; “K_{D}” refers to the equilibrium dissociation constant (M); “kₐ” refers to the association rate or constant (1/Ms); “k_{d}” refers to dissociation rate or constant (1/s); “Rₘₐₓ” refers to theoretical Rₘₐₓ or calculated maximum observable signal (relative units); “SA” refers to surface activity (%); and “Ratio” refers to the calculated ratio of the variant antibody K_{D}/burosumab K_{D}. For clarity, each antibody represented by Antibody Structure ID numbers UGX205, UGX201, UGX144, UGX157, UGX202, UGX175, UGX206, UGX182, UGX156, UGX203, UGX126, UGX145, and UGX204 in Table 19 has the parental burosumab heavy chain sequence (SEQ ID NO: 1) and light chain sequence (SEQ ID NO: 2) save for the indicated amino acid changes relative to those sequences.

TABLE 19
Select antibody variants with a K_{D} ratio (calculated ratio of the variant
antibody K_{D}/burosumab K_{D}) greater than or equal to 2.

| | AA | VH | VL | | | | | | |
|---|---|---|---|---|---|---|---|---|---|
| AS ID | Change(s) | ID | ID | K_{D} | kₐ | k_{d} | RU | SA | Ratio |
| UGX205 | HC:S59E, | VH- | VL- | 4.07E−12 | 1.53E+07 | 6.25E−05 | 59.2 | 93.9 | 5 |
| | LC:Y94D | 031 | 066 | | | | | | |
| UGX201 | HC:S59E, | VH- | VL- | 5.17E−12 | 1.12E+07 | 5.81E−05 | 30.9 | 83.4 | 4 |
| | LC:N92F | 031 | 050 | | | | | | |
| UGX144 | LC:Y94D | VH- | VL- | 5.77E−12 | 1.09E+07 | 6.31E−05 | 65.4 | 89.5 | 4 |
| | | 001 | 066 | | | | | | |
| UGX157 | HC:I54Y | VH- | VL- | 7.58E−12 | 1.49E+07 | 1.13E−04 | 71.4 | 89.1 | 3 |
| | | 029 | 001 | | | | | | |
| UGX202 | HC:S59E, | VH- | VL- | 7.59E−12 | 1.29E+07 | 9.80E−05 | 56.7 | 91.6 | 3 |
| | LC:N92Y | 031 | 054 | | | | | | |
| UGX175 | HC:A103S | VH- | VL- | 7.72E−12 | 1.24E+07 | 9.61E−05 | 73.1 | 86.8 | 3 |
| | | 061 | 001 | | | | | | |
| UGX206 | HC:S59E, | VH- | VL- | 7.87E−12 | 6.25E+06 | 4.92E−05 | 20.1 | 107.6 | 3 |
| | LC:Y94E | 031 | 067 | | | | | | |
| UGX182 | HC:S59D, | VH- | VL- | 9.47E−12 | 1.49E+07 | 1.41E−04 | 36.5 | 100.5 | 2 |
| | HC:V101I | 055 | 001 | | | | | | |
| UGX156 | HC:I54F | VH- | VL- | 1.05E−11 | 5.94E+06 | 6.25E−05 | 43.2 | 105.9 | 2 |
| | | 028 | 001 | | | | | | |
| UGX203 | HC:S59E, | VH- | VL- | 1.15E−11 | 1.26E+07 | 1.45E−04 | 45.9 | 81.4 | 2 |
| | LC:S52D | 031 | 027 | | | | | | |
| UGX126 | LC:S52D | VH- | VL- | 1.24E−11 | 1.05E+07 | 1.30E−04 | 56.2 | 86.5 | 2 |
| | | 001 | 027 | | | | | | |
| UGX145 | LC:Y94E | VH- | VL- | 1.33E−11 | 6.42E+06 | 8.52E−05 | 46.8 | 87.4 | 2 |
| | | 001 | 067 | | | | | | |
| UGX204 | HC:S59E, | VH- | VL- | 1.33E−11 | 1.40E+07 | 1.86E−04 | 46.6 | 90.4 | 2 |
| | LC:S52E | 031 | 030 | | | | | | |
| Burosumab | — | VH- | VL- | 2.08E−11 | 7.22E+06 | 1.57E−04 | 66.2 | 88.8 | 1 |
| | | 001 | 001 | | | | | | |

The SPR data suggest multiple paratope residues are acting through various PG-4T mechanisms to support burosumab:FGF23 binding. In particular, amino acid substitutions S52D/E, N92F/Y, and Y94D/E to the parental burosumab light chain sequence and 154F/Y, S59D/E, V101I, and A103S to the parental burosumab heavy chain sequence significantly affect affinity of the antibody to FGF23, predominantly by slowing the off-rate k_{d}.

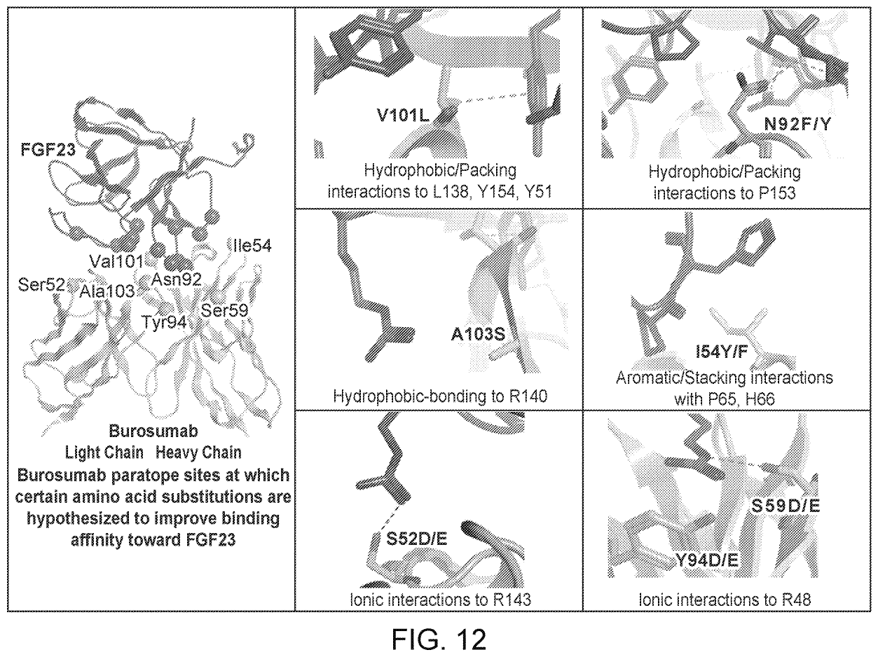

Without wishing to be bound by theory, the present inventors hypothesize that: light chain Y94D/E substitutions may form ionic interactions to epitope residue Arg48; heavy chain S59E and light chain Y94D/E substitutions may work in concert to stabilize sandwiching interactions with epitope residue Arg48; light chain N92F/Y substitutions appear to strengthen hydrophobic packing interactions with epitope residues, such as Pro153, particularly with heavy chain S59E substitution; heavy chain A103S substitution likely introduces a hydrogen bond with a nearby epitope residue, including Arg140; heavy chain S59E and light chain S52D/E substitutions may form ionic interactions to epitope residues Arg48 and Arg143, respectively; heavy chain V101I substitution likely promotes stronger hydrophobic packing interaction with epitope residues Tyr51, L138 and Tyr154; and heavy chain I54F/Y substitutions may promote stacking interaction with epitope residue His66 (FIG. 12).

### Example 11. Characterization of Selected Variant Antibodies Against cynoFGF23

Binding kinetics of 13 antibody variants shown in Table 19 were characterized against cynomolgus monkey (Macaca fascicularis) FGF23 (R179Q) (i.e., “cynoFGF23”) (SEQ ID NO: 372). Antibody variants were expressed at small scale (3 ml) and screened by SPR analysis at a single concentration (100 nM). Experimental setup of SPR analysis for CRC: SPR device—8K+; chip preparation—CM5 hIgG chip: channels—1-8; immobilization concentration—1.5 ug/ml; immobilization method—capture; flow rate—5 μl/min; contact time—60 s; running buffer—1×HBS-EP+pH 7.4; temperature—20° C.: flow rate—50 μl/min; association time—120 s; dissociation time—1800 s; antigen—cynoFGF23(R179Q): top concentration—0.1 μM, 9-pt 3-fold Single Cycle Kinetics. Results are provided in Table 20, which shows K_{D} and calculated ratio of the variant antibody K_{D}/burosumab K_{D} for each antibody variant against human FGF23 and cynoFGF23.

TABLE 20
Characterization of selected antibody variants against cynoFGF23

| | | | | Cynomolgus monkey | |
|---|---|---|---|---|---|
| | | Human FGF23 | | FGF23 | |
| AS ID | AA Change | K_{D} (M) | K_{D} ratio | K_{D} (M) | K_{D} ratio |
| UGX205 | S59E/Y94D | 4.07E−12 | 5 | 2.57E−12 | 9 |
| UGX201 | S59E/N92F | 5.17E−12 | 4 | 1.01E−11 | 2 |
| UGX144 | Y94D | 5.77E−12 | 4 | 6.86E−12 | 3 |
| UGX157 | I54Y | 7.58E−12 | 3 | 5.73E−12 | 4 |
| UGX202 | S59E/N92Y | 7.59E−12 | 3 | 6.93E−12 | 3 |
| UGX175 | A103S | 7.72E−12 | 3 | 6.17E−12 | 4 |
| UGX206 | S59E/Y94E | 7.87E−12 | 3 | 7.16E−12 | 3 |
| UGX182 | S59D/V101I | 9.47E−12 | 2 | 1.63E−11 | 1 |
| UGX156 | I54F | 1.05E−11 | 2 | 4.15E−12 | 6 |
| UGX203 | S59E/S52D | 1.15E−11 | 2 | 2.29E−11 | 1 |
| UGX126 | S52D | 1.24E−11 | 2 | 1.03E−11 | 2 |
| UGX145 | Y94E | 1.33E−11 | 2 | 1.29E−11 | 2 |
| UGX204 | S59E/S52E | 1.33E−11 | 2 | 1.60E−11 | 1 |
| UGX139 | N92Y | 1.64E−11 | 1 | 1.62E−11 | 1 |

Binding of selected antibody variants to cynoFGF23 was comparable to that observed to human FGF23.

### Example 12. Confirmation of HDX-MS Epitope/Paratope Mapping by SPR Analysis

HDX-MS epitope and paratope mapping data, described in Examples 2 and 3 respectively, were reevaluated in light of SPR data described in Example 10 to confirm effects of key residue changes within HDX-MS protected regions. Epitope mapping identified three putative epitopes (protected regions 1-3 in Table 3) and paratope mapping identified six regions potentially involved in antigen binding (see Table 4).

SPR data confirmed burosumab light chain residues S52 in light chain paratope region 2 (see Table 4) and 154 and S59 in heavy chain paratope region 2 (see Table 4) are key residues within HDX-MS protected regions of burosumab, suggesting these residues are important mediators of burosumab-FGF23 binding.

SPR data also confirmed burosumab light chain residues S52, N92, and Y94 and burosumab heavy chain residues S59 and V101 are key residues interfacing with FGF23 protected regions 1-3 (Table 3).

Thus, of the 7 key burosumab residues determined by SPR (light chain S52, N92, Y94 and heavy chain 154, S59, V101, and A103) to be substitutable to improve burosumab affinity, 6 of them were also identified by HDX-MS epitope/paratope mapping. This shows strong correlation and agreement among results from initial HDX-MS studies and subsequent crystal structure-based modeling and antibody variant design.

### Example 13. In Silico Modeling of Additional Antibody Variants Based on SPR Analysis

Examples 10 through 12 identify and confirm at least 7 burosumab residues (light chain S52, N92, Y94 and heavy chain 154, S59, V101, and A103) that are substitutable to improve affinity of the antibody to FGF23. These key burosumab residues were further evaluated by in silico modeling to identify additional antibody variants with equivalent or improved affinity compared to parental burosumab and/or variants identified in Table 18 and Table 19.

Briefly, all 7 key burosumab residues were interrogated using predictive mutagenesis by in silico modeling based on the burosumab Fab:FGF23 crystal structure and SPR data. Specifically, antibody variants from Table 18 and Table 19 having a K_{D} ratio (calculated ratio of the variant antibody K_{D}/burosumab K_{D}) greater than 2 (i.e., 3 or greater; UGX205, UGX201, UGX202, and UGX206), were analyzed using Molecular Operating Environment “MOE” (2022 February) to define the structural determinants of binding (i.e., which single or pairs of residues may be working synergistically to improve affinity). All possible combinations of amino acid changes to the 7 key burosumab residues of these antibodies were generated computationally. Certain amino acid changes (per amino acid position) that were consistent with initial empirical SPR data were then selected for further analysis. All amino acid variant combinations predicted to improve affinity relative to parental burosumab from a structural/computational standpoint are shown in Table 21, wherein: “AS ID” refers to “Antibody Structure ID”, a unique identifier for each antibody as defined herein; “AA Change(s)” refers to amino acid residue changes relative to either the parental burosumab heavy chain (“HC”) variable region amino acid sequence (SEQ ID NO: 3) or the parental burosumab light chain (“LC”) variable region amino acid sequence (SEQ ID NO: 4), or both; “ΔΔG” refers to calculated dAffinity (kcal/mol); “ΔΔS” refers to calculated dStability (kcal/mol); and “Rationale” refers to key observations noted during the MOE assessment.

TABLE 21
Structural computational evaluation of additional antibody variants

| | AA Change(s) | AA Change(s) | | | |
|---|---|---|---|---|---|
| AS ID | LC | HC | ΔΔG | ΔΔS | Rationale |
| UGX205 | Y94D | S59E | 4.07 | 1.98 | |
| UGX144 | Y94D | | 2.35 | 1.07 | |
| UGX201 | N92F | S59E | −5.8 | 1.87 | In silico derived dAffinity energies |
| | | | | | consistent with SPR |
| UGX157 | | I54Y | 2.65 | 1.80 | |
| UGX175 | | A103S | −0.09 | 0.23 | In silico derived dAffinity energies |
| | | | | | consistent with SPR |
| UGX206 | Y94E | S59E | −1.82 | 3.43 | In silico derived dAffinity energies |
| | | | | | consistent with SPR |
| UGX202 | N92Y | S59E | 1.34 | 0.24 | |
| UGX156 | | I54F | 1.95 | 2.05 | |
| UGX182 | | S59D, V101I | −0.49 | 0.56 | In silico derived dAffinity energies |
| | | | | | consistent with SPR |
| UGX203 | S52D | S59E | 0.56 | 1.8 | |
| UGX126 | S52D | | −0.50 | 1.28 | In silico derived dAffinity energies |
| | | | | | consistent with SPR |
| UGX145 | Y94E | | 2.58 | 1.05 | |
| UGX204 | S52E | S59E | 0.53 | 1.8 | |
| UGX301 | Y94D | S59D | 2.21 | 1.57 | S59D/E/Q likely contacts nearby R48, |
| UGX302 | Y94D | S59Q | 1.88 | 1.08 | which is also engaged by Y94D, creating |
| UGX303 | Y94E | S59D | 3.05 | 1.31 | an electrostatic bridge. Provided the |
| UGX304 | Y94E | S59Q | −5.5 | 2.24 | correct geometry can be achieved, these |
| UGX305 | Y94Q | S59D | 2.23 | 1.19 | two interactions may significantly |
| UGX306 | Y94Q | S59E | −3.17 | 2.93 | increase affinity. |
| UGX307 | Y94Q | S59Q | −3.81 | 2.75 | |
| UGX308 | | S59E, I54Y | −1.82 | 2.24 | I54Y likely contacts nearby P65 and |
| UGX309 | | S59D, I54Y | −3.2 | 2.27 | H66. S59E likely contacts nearby R48. |
| UGX310 | | S59Q, I54Y | 1.1 | 1.70 | These two interactions may increase the |
| | | | | | KD ratio and create an additive effect. |
| UGX311 | Y94D | I54Y | 1.83 | 1.99 | I54Y likely contacts nearby P65 and |
| UGX312 | Y94E | I54Y | 3.30 | 2.04 | H66. YA4D/E/Q likely contacts nearby |
| UGX313 | Y94Q | I54Y | 2.32 | 1.84 | R48. These two interactions may |
| | | | | | increase the KD ratio in a synergistic |
| | | | | | manner. |
| UGX314 | N92Y | S59D | −5.05 | 1.38 | S59D/E/Q likely contacts nearby R48 |
| UGX315 | N92Y | S59Q | 1.34 | 1.98 | and is the main driver of affinity |
| UGX316 | N92F | S59D | −5.46 | 1.10 | increase. An aromatic residue at position |
| UGX317 | N92F | S59Q | −5.75 | 1.33 | N92 (ie., F or Y) can further stabilize |
| UGX318 | N92L | S59D | −2.07 | 1.10 | the conformation of R48 by two possible |
| UGX319 | N92L | S59E | 1.17 | 1.87 | mechanisms: l. Pushing the backbone of |
| UGX320 | N92L | S59Q | −4.31 | 1.33 | nearby A47 which in turn may allow |
| UGX321 | N92I | S59D | −2.78 | 1.30 | more space for R48 to form a stacking |
| UGX322 | N92I | S59E | −1.79 | 1.50 | interaction with F95. 2. Sterically |
| UGX323 | N92I | S59Q | −4.99 | 0.87 | preventing a potential R48 swing-back. |
| UGX324 | N92T | S59D | 1.90 | 1.98 | In either scenario N92Y or F may |
| UGX325 | N92T | S59E | 0.01 | 2.91 | interact with nearby P153 from the |
| UGX326 | N92T | S59Q | −2.64 | 1.93 | antigen to complete the local network of |
| | | | | | interactions. |
| UGX327 | | I54A | 3.55 | 1.96 | Consistent with existing experimental |
| UGX328 | | I54L | 2.44 | 1.61 | data provided herein. |
| UGX329 | | I54V | 2.53 | 1.52 | |
| UGX330 | | I54R | 1.22 | 1.60 | |
| UGX331 | | I54D | 4.31 | 2.76 | |
| UGX332 | | I54G | 5.4 | 2.73 | |
| UGX333 | | I54H | 3.57 | 2.54 | |
| UGX334 | | I54K | 2.73 | 2.47 | |
| UGX335 | | I54M | 1.53 | 1.09 | |
| UGX336 | | I54N | 3.29 | 2.29 | |
| UGX337 | | I54P | 6.25 | 2.50 | |
| UGX338 | | I54W | 1.53 | 1.63 | |
| UGX339 | | S59A | 0.56 | 0.42 | Allows confirmation that S59 to acidic is |
| UGX340 | | S59R | −1.23 | 0.51 | necessary to lock in R48. |
| UGX341 | | S59N | −0.38 | 1.08 | |
| UGX342 | | S59G | 0.74 | 0.82 | |
| UGX343 | | S59H | −1.34 | 1.00 | |
| UGX344 | | S59K | −0.36 | 1.23 | |
| UGX345 | | S59M | −1.15 | 0.38 | |
| UGX346 | | S59P | −2.83 | 0.55 | |
| UGX347 | | S59W | −3.39 | −0.53 | |
| UGX348 | Y94A | | 3.18 | 1.07 | Consistent with existing experimental |
| UGX349 | Y94N | | 1.82 | 0.99 | data provided herein. |
| UGX350 | Y94G | | 2.87 | 1.78 | |
| UGX351 | Y94H | | 1.38 | 1.12 | |
| UGX352 | Y94I | | 2.77 | −0.18 | |
| UGX353 | Y94L | | 2.91 | 0.14 | |
| UGX354 | Y94K | | 6.05 | 1.56 | |
| UGX355 | Y94M | | 3.22 | 1.71 | |
| UGX356 | Y94P | | 1.32 | 1.03 | |
| UGX357 | Y94W | | −1.52 | 0.90 | |
| UGX358 | Y94V | | 1.67 | 0.31 | |
| UGX359 | N92A | | 4.44 | 1.36 | Consistent with existing experimental |
| UGX360 | N92R | | 0.80 | 0.37 | data provided herein. |
| UGX361 | N92G | | 5.24 | 2.08 | |
| UGX362 | N92H | | −1.20 | 0.79 | |
| UGX363 | N92I | | 0.09 | −0.51 | |
| UGX364 | N92L | | −0.55 | −0.68 | |
| UGX365 | N92K | | 1.19 | 0.91 | |
| UGX366 | N92M | | −1.51 | 0.20 | |
| UGX367 | N92P | | 3.72 | 1.59 | |
| UGX368 | N92W | | −1.16 | 0.45 | |
| UGX369 | N92V | | 0.73 | −0.11 | |
| UGX370 | | A103I | −2.64 | −0.19 | Difficult to capture in silico but could be |
| UGX371 | | A103L | 1.0 | 1.98 | beneficial to improve the stability of the |
| UGX372 | | A103R | 5.39 | −0.29 | VH:VL interface with a positive effect |
| UGX373 | | A103N | −0.27 | 0.58 | on FGF23 binding and/or provide a |
| UGX374 | | A103G | 0.02 | 1.62 | direct contact to nearby Arg140. Because |
| UGX375 | | A103H | 2.93 | 0.09 | of potential dial effects, may also be |
| UGX376 | | A103K | 0.23 | 0.57 | beneficial to test with one of the additive |
| UGX377 | | A103M | −0.14 | 0.56 | combinations with position 103 in |
| UGX378 | | A103P | −0.04 | −0.12 | addition to a single mutation (see |
| UGX379 | | A103W | 7.92 | 0.41 | UGX418 to UGX423). |
| UGX380 | | A103V | 0.8 | 0.84 | |
| UGX381 | S52D | S59Q | −2.42 | 1.13 | A shorter acidic residue at position 59 |
| UGX382 | S52D | S59D | 0.03 | 1.77 | and/or 52 may be more compatible with |
| UGX383 | S52E | S59Q | −2.41 | 1.06 | the contacting residues on the epitope. |
| UGX384 | S52E | S59D | 0.01 | 1.67 | |
| UGX385 | S52Q | S59E | 0.42 | 1.48 | |
| UGX386 | S52A | | −0.2 | 0.57 | Consistent with existing experimental |
| UGX387 | S52R | | −0.5 | −0.32 | data provided herein. |
| UGX388 | S52N | | −0.13 | 0.97 | |
| UGX389 | S52G | | −0.01 | 0.84 | |
| UGX390 | S52H | | −3.0 | 0.44 | |
| UGX391 | S52I | | −0.57 | −0.38 | |
| UGX392 | S52L | | −0.91 | −0.23 | |
| UGX393 | S52K | | −1.7 | 0.21 | |
| UGX394 | S52M | | −1.91 | −0.10 | |
| UGX395 | S52P | | 0.35 | 1.57 | |
| UGX396 | S52W | | −3.18 | −0.56 | |
| UGX397 | S52V | | 0.21 | −0.14 | |
| UGX398 | | S59E, V101I | −1.0 | 0.40 | Likely additive and >3x KD |
| UGX399 | | S59D, V101L | 0.81 | 1.06 | improvement. |
| UGX400 | | S59Q, V101I | −6.8 | 0.33 | |
| UGX401 | | S59D, V101S | 2.87 | 2.44 | |
| UGX402 | | S59E, V101S | 2.46 | 2.21 | |
| UGX403 | | V101A | 4.38 | 1.97 | Consistent with existing experimental |
| UGX404 | | V101R | −3.47 | 0.31 | data provided herein. |
| UGX405 | | V101N | 1.34 | 2.00 | |
| UGX406 | | V101D | −0.69 | 2.50 | |
| UGX407 | | V101Q | 0.55 | 1.46 | |
| UGX408 | | V101E | −0.31 | 1.73 | |
| UGX409 | | V101G | 6.56 | 2.84 | |
| UGX410 | | V101H | −1.65 | 1.69 | |
| UGX411 | | V101K | −3.46 | 0.91 | |
| UGX412 | | V101M | −1.42 | 0.77 | |
| UGX413 | | V101P | 1.70 | 1.95 | |
| UGX414 | | V101S | 2.68 | 2.22 | |
| UGX415 | | V101W | −2.91 | 0.13 | |
| UGX416 | | I54Y, A103S | 3.00 | 1.80 | Likely additive and >3x KD |
| UGX417 | | I54F, A103S | 3.00 | 1.39 | improvement. |
| UGX418 | Y94D | A103S | 0.46 | 0.24 | |
| UGX419 | Y94E | A103S | −0.55 | 0.39 | |
| UGX420 | N92F | A103S | −1.02 | −0.72 | |
| UGX421 | N92Y | A103S | 0.95 | −0.10 | |
| UGX422 | S52D | A103S | −1.76 | 1.29 | |
| UGX423 | S52E | A103S | −0.7 | 1.24 | |
| UGX424 | | S59D, A103S | −0.36 | 0.39 | |
| UGX425 | | S59E, A103S | −0.23 | 0.53 | |
| UGX426 | | S59Q, A103S | −3.10 | −0.32 | |
| UGX427 | N92Y | I54Y | 1.19 | 1.38 | |
| UGX428 | N92F | I54Y | 1.00 | 1.00 | |
| UGX429 | | V101I, I54F | −1.01 | 1.58 | |
| UGX430 | | V101I, I54Y | −1.59 | 1.24 | |

To summarize, mutational solutions from the lead antibody variants (e.g., Table 19) were analyzed using structural computational methods and a set of new variants was generated with designs predicted to be of similar or superior affinity. The additional designs included several burosumab antibody variants predicted to have improved affinity compared to parental burosumab.

### Example 14. Data Summary

The studies described herein sought to rationally design burosumab antibody variants with improved affinity to FGF23 by introducing one or more amino acid change(s) to either the heavy chain amino acid sequence, the light chain amino acid sequence, or both the heavy chain amino acid sequence and the light chain amino acid sequence.

Amino acid residue changes were modeled based on input from epitope and paratope mapping studies (Examples 2 and 3), molecular docking and predictive mutagenesis studies (Example 4), structural analysis by crystallography (Example 7) and predictive mutagenesis based on the solved crystal structure (Example 8), refinement of antibody variant designs to identify most promising antibody structural variants having 2-fold or greater improvements in affinity to human FGF23 and in vitro testing of selected variants by SPR analysis (Examples 9 and 10).

The studies reported herein further characterized certain lead antibody variants for affinity to cynoFGF23 (Example 11) and identified additional antibody variants by computationally interrogating alternative amino acid substitutions at the key residues identified as important for modulating burosumab-FGF23 binding (Example 13).

Table 22 lists unique burosumab antibody variants designed according to the studies described herein. In Table 22, each variant antibody is ascribed a unique “AS ID” number. Further, each antibody variant has one or more light chain amino acid change (“LC AA Change”) relative to parental burosumab light chain amino acid sequence of SEQ ID NO: 2 or has one or more light chain variable region amino acid change (“VL AA Change”) relative to parental burosumab light chain variable region amino acid sequence of SEQ ID NO: 4, and/or has one or more heavy chain amino acid change (“HC AA Change”) relative to parental burosumab heavy chain amino acid sequence of SEQ ID NO: 1 or has one or more heavy chain variable region amino acid change (“VH AA Change) relative to parental burosumab heavy chain variable region amino acid sequence of SEQ ID NO: 3. In Table 22, the “AKA” column refers to alternative identifiers assigned to certain of the AS ID's provided herein. Sequence identification numbers (“SID #s”) are provided for respective heavy chain (HC), light chain (LC), heavy chain variable region (VH), and light chain variable region (VL) amino acid sequences disclosed herein.

TABLE 22
Summary of burosumab antibody variants rationally
designed to have improved affinity to human FGF23

| | LC/VL | HC/VH | | HC | LC | VH | VL |
|---|---|---|---|---|---|---|---|
| | AA | AA | | SID | SID | SID | SID |
| AS ID | Change | Change | AKA | # | # | # | # |
| UGX001 | Q27F | parental | — | 654 | 655 | 656 | 657 |
| UGX002 | Q27R | parental | — | 658 | 659 | 660 | 661 |
| UGX003 | G28D | parental | — | 662 | 663 | 664 | 665 |
| UGX004 | G28Q | parental | — | 666 | 667 | 668 | 669 |
| UGX005 | G28R | parental | — | 670 | 671 | 672 | 673 |
| UGX006 | G28Y | parental | — | 674 | 675 | 676 | 677 |
| UGX007 | S30D | parental | UGX103 | 678 | 679 | 680 | 681 |
| UGX008 | S30F | parental | UGX105 | 682 | 683 | 684 | 685 |
| UGX009 | S30R | parental | — | 686 | 687 | 688 | 689 |
| UGX010 | S30Y | parental | UGX108 | 690 | 691 | 692 | 693 |
| UGX011 | S31F | parental | UGX111 | 694 | 695 | 696 | 697 |
| UGX012 | S31Y | parental | UGX113 | 698 | 699 | 700 | 701 |
| UGX013 | D50E | parental | — | 702 | 703 | 704 | 705 |
| UGX014 | D50Q | parental | UGX120 | 706 | 707 | 708 | 709 |
| UGX015 | D50R | parental | UGX121 | 710 | 711 | 712 | 713 |
| UGX016 | D50Y | parental | — | 714 | 715 | 716 | 717 |
| UGX017 | S52E | parental | UGX127 | 718 | 719 | 720 | 721 |
| UGX018 | S52Q | parental | — | 722 | 723 | 724 | 725 |
| UGX019 | S52R | parental | UGX387 | 726 | 727 | 728 | 729 |
| UGX020 | S52Y | parental | — | 730 | 731 | 732 | 733 |
| UGX021 | S53R | parental | UGX132 | 734 | 735 | 736 | 737 |
| UGX022 | S53Y | parental | UGX133 | 738 | 739 | 740 | 741 |
| UGX023 | L54E | parental | — | 742 | 743 | 744 | 745 |
| UGX024 | L54Y | parental | — | 746 | 747 | 748 | 749 |
| UGX025 | E55R | parental | — | 750 | 751 | 752 | 753 |
| UGX026 | E55Y | parental | — | 754 | 755 | 756 | 757 |
| UGX027 | S67E | parental | — | 758 | 759 | 760 | 761 |
| UGX028 | S67R | parental | — | 762 | 763 | 764 | 765 |
| UGX029 | S67Y | parental | — | 766 | 767 | 768 | 769 |
| UGX030 | T69R | parental | — | 770 | 771 | 772 | 773 |
| UGX031 | T69Y | parental | — | 774 | 775 | 776 | 777 |
| UGX032 | F91E | parental | — | 778 | 779 | 780 | 781 |
| UGX033 | F91R | parental | — | 782 | 783 | 784 | 785 |
| UGX034 | N92E | parental | UGX136 | 786 | 787 | 788 | 789 |
| UGX035 | N92F | parental | UGX137 | 790 | 791 | 792 | 793 |
| UGX036 | N92Q | parental | — | 794 | 795 | 796 | 797 |
| UGX037 | N92R | parental | UGX360 | 798 | 799 | 800 | 801 |
| UGX038 | N92Y | parental | UGX139 | 802 | 803 | 804 | 805 |
| UGX039 | D93F | parental | UGX140 | 806 | 807 | 808 | 809 |
| UGX040 | D93R | parental | — | 810 | 811 | 812 | 813 |
| UGX041 | D93Y | parental | — | 814 | 815 | 816 | 817 |
| UGX042 | Y94E | parental | UGX145 | 818 | 819 | 820 | 821 |
| UGX043 | Y94R | parental | — | 822 | 823 | 824 | 825 |
| UGX044 | parental | T28Y | — | 826 | 827 | 828 | 829 |
| UGX045 | parental | T30K | — | 830 | 831 | 832 | 833 |
| UGX046 | parental | H32K | — | 834 | 835 | 836 | 837 |
| UGX047 | parental | H32Y | — | 838 | 839 | 840 | 841 |
| UGX048 | parental | Y33D | — | 842 | 843 | 844 | 845 |
| UGX049 | parental | Y33L | — | 846 | 847 | 848 | 849 |
| UGX050 | parental | Y33R | — | 850 | 851 | 852 | 853 |
| UGX051 | parental | N52E | — | 854 | 855 | 856 | 857 |
| UGX052 | parental | N52Q | — | 858 | 859 | 860 | 861 |
| UGX053 | parental | N52R | — | 862 | 863 | 864 | 865 |
| UGX054 | parental | S55E | — | 866 | 867 | 868 | 869 |
| UGX055 | parental | S55F | — | 870 | 871 | 872 | 873 |
| UGX056 | parental | S55I | — | 874 | 875 | 876 | 877 |
| UGX057 | parental | S55Q | — | 878 | 879 | 880 | 881 |
| UGX058 | parental | S55R | — | 882 | 883 | 884 | 885 |
| UGX059 | parental | S55Y | — | 886 | 887 | 888 | 889 |
| UGX060 | parental | S57E | — | 890 | 891 | 892 | 893 |
| UGX061 | parental | S57F | — | 894 | 895 | 896 | 897 |
| UGX062 | parental | S57R | — | 898 | 899 | 900 | 901 |
| UGX063 | parental | S57Y | — | 902 | 903 | 904 | 905 |
| UGX064 | parental | T58F | — | 906 | 907 | 908 | 909 |
| UGX065 | parental | T58R | — | 910 | 911 | 912 | 913 |
| UGX066 | parental | S59F | UGX160 | 914 | 915 | 916 | 917 |
| UGX067 | parental | S59R | UGX340 | 918 | 919 | 920 | 921 |
| UGX068 | parental | S59Y | UGX166 | 922 | 923 | 924 | 925 |
| UGX069 | parental | N60E | — | 926 | 927 | 928 | 929 |
| UGX070 | parental | N60R | — | 930 | 931 | 932 | 933 |
| UGX071 | parental | N60Y | — | 934 | 935 | 936 | 937 |
| UGX072 | parental | A61F | — | 938 | 939 | 940 | 941 |
| UGX073 | parental | A61R | — | 942 | 943 | 944 | 945 |
| UGX074 | parental | A61Y | — | 946 | 947 | 948 | 949 |
| UGX075 | parental | Q62L | — | 950 | 951 | 952 | 953 |
| UGX076 | parental | Q62R | — | 954 | 955 | 956 | 957 |
| UGX077 | parental | Q62Y | — | 958 | 959 | 960 | 961 |
| UGX078 | parental | Q65E | — | 962 | 963 | 964 | 965 |
| UGX079 | parental | Q65R | — | 966 | 967 | 968 | 969 |
| UGX080 | parental | Q65Y | — | 970 | 971 | 972 | 973 |
| UGX081 | parental | D99F | — | 974 | 975 | 976 | 977 |
| UGX082 | parental | D99R | — | 978 | 979 | 980 | 981 |
| UGX083 | parental | D99Y | — | 982 | 983 | 984 | 985 |
| UGX084 | parental | I100E | UGX168 | 986 | 987 | 988 | 989 |
| UGX085 | parental | I100F | UGX169 | 990 | 991 | 992 | 993 |
| UGX086 | parental | I100Y | — | 994 | 995 | 996 | 997 |
| UGX087 | parental | V101E | UGX408 | 998 | 999 | 1000 | 1001 |
| UGX088 | parental | V101F | UGX170 | 1002 | 1003 | 1004 | 1005 |
| UGX089 | parental | V101L | UGX172 | 1006 | 1007 | 1008 | 1009 |
| UGX090 | parental | V101Q | UGX407 | 1010 | 1011 | 1012 | 1013 |
| UGX091 | parental | V101R | UGX404 | 1014 | 1015 | 1016 | 1017 |
| UGX092 | parental | D102E | — | 1018 | 1019 | 1020 | 1021 |
| UGX093 | parental | D102F | — | 1022 | 1023 | 1024 | 1025 |
| UGX094 | parental | D102K | — | 1026 | 1027 | 1028 | 1029 |
| UGX095 | parental | D102R | — | 1030 | 1031 | 1032 | 1033 |
| UGX096 | parental | D102Y | — | 1034 | 1035 | 1036 | 1037 |
| UGX101 | I29F | parental | — | 1038 | 1039 | 1040 | 1041 |
| UGX102 | I29L | parental | — | 1042 | 1043 | 1044 | 1045 |
| UGX104 | S30E | parental | — | 1046 | 1047 | 1048 | 1049 |
| UGX106 | S30L | parental | — | 1050 | 1051 | 1052 | 1053 |
| UGX107 | S30Q | parental | — | 1054 | 1055 | 1056 | 1057 |
| UGX109 | S31D | parental | — | 1058 | 1059 | 1060 | 1061 |
| UGX110 | S31E | parental | — | 1062 | 1063 | 1064 | 1065 |
| UGX112 | S31Q | parental | — | 1066 | 1067 | 1068 | 1069 |
| UGX114 | A32F | parental | — | 1070 | 1071 | 1072 | 1073 |
| UGX115 | A32V | parental | — | 1074 | 1075 | 1076 | 1077 |
| UGX116 | V34F | parental | — | 1078 | 1079 | 1080 | 1081 |
| UGX117 | V34I | parental | — | 1082 | 1083 | 1084 | 1085 |
| UGX118 | D50F | parental | — | 1086 | 1087 | 1088 | 1089 |
| UGX119 | D50L | parental | — | 1090 | 1091 | 1092 | 1093 |
| UGX122 | D50S | parental | — | 1094 | 1095 | 1096 | 1097 |
| UGX123 | D50T | parental | — | 1098 | 1099 | 1100 | 1101 |
| UGX124 | D50V | parental | — | 1102 | 1103 | 1104 | 1105 |
| UGX125 | A51V | parental | — | 1106 | 1107 | 1108 | 1109 |
| UGX126 | S52D | parental | — | 1110 | 1111 | 1112 | 1113 |
| UGX128 | S52F | parental | — | 1114 | 1115 | 1116 | 1117 |
| UGX129 | S53D | parental | — | 1118 | 1119 | 1120 | 1121 |
| UGX130 | S53E | parental | — | 1122 | 1123 | 1124 | 1125 |
| UGX131 | S53F | parental | — | 1126 | 1127 | 1128 | 1129 |
| UGX134 | F91Y | parental | — | 1130 | 1131 | 1132 | 1133 |
| UGX135 | N92D | parental | — | 1134 | 1135 | 1136 | 1137 |
| UGX138 | N92S | parental | — | 1138 | 1139 | 1140 | 1141 |
| UGX141 | D93Q | parental | — | 1142 | 1143 | 1144 | 1145 |
| UGX142 | D93S | parental | — | 1146 | 1147 | 1148 | 1149 |
| UGX143 | D93T | parental | — | 1150 | 1151 | 1152 | 1153 |
| UGX144 | Y94D | parental | — | 1154 | 1155 | 1156 | 1157 |
| UGX146 | Y94F | parental | — | 1158 | 1159 | 1160 | 1161 |
| UGX147 | Y94S | parental | — | 1162 | 1163 | 1164 | 1165 |
| UGX148 | parental | T30F | — | 1166 | 1167 | 1168 | 1169 |
| UGX149 | parental | N31D | — | 1170 | 1171 | 1172 | 1173 |
| UGX150 | parental | N31E | — | 1174 | 1175 | 1176 | 1177 |
| UGX151 | parental | N31F | — | 1178 | 1179 | 1180 | 1181 |
| UGX152 | parental | N31Q | — | 1182 | 1183 | 1184 | 1185 |
| UGX153 | parental | N31R | — | 1186 | 1187 | 1188 | 1189 |
| UGX154 | parental | N52F | — | 1190 | 1191 | 1192 | 1193 |
| UGX155 | parental | N52Y | — | 1194 | 1195 | 1196 | 1197 |
| UGX156 | parental | I54F | — | 1198 | 1199 | 1200 | 1201 |
| UGX157 | parental | I54Y | — | 1202 | 1203 | 1204 | 1205 |
| UGX158 | parental | S59D | — | 1206 | 1207 | 1208 | 1209 |
| UGX159 | parental | S59E | — | 1210 | 1211 | 1212 | 1213 |
| UGX161 | parental | S59I | — | 1214 | 1215 | 1216 | 1217 |
| UGX162 | parental | S59L | — | 1218 | 1219 | 1220 | 1221 |
| UGX163 | parental | S59Q | — | 1222 | 1223 | 1224 | 1225 |
| UGX164 | parental | S59T | — | 1226 | 1227 | 1228 | 1229 |
| UGX165 | parental | S59V | — | 1230 | 1231 | 1232 | 1233 |
| UGX167 | parental | I100D | — | 1234 | 1235 | 1236 | 1237 |
| UGX171 | parental | V101I | — | 1238 | 1239 | 1240 | 1241 |
| UGX173 | parental | A103D | — | 1242 | 1243 | 1244 | 1245 |
| UGX174 | parental | A103F | — | 1246 | 1247 | 1248 | 1249 |
| UGX175 | parental | A103S | — | 1250 | 1251 | 1252 | 1253 |
| UGX176 | parental | A103T | — | 1254 | 1255 | 1256 | 1257 |
| UGX177 | S31E, | parental | — | 1258 | 1259 | 1260 | 1261 |
| | D50F | | | | | | |
| UGX178 | parental | N31F, | — | 1262 | 1263 | 1264 | 1265 |
| | | S59F | | | | | |
| UGX179 | D50F, | parental | — | 1266 | 1267 | 1268 | 1269 |
| | S53E | | | | | | |
| UGX180 | S31F, | parental | — | 1270 | 1271 | 1272 | 1273 |
| | S52E | | | | | | |
| UGX181 | D50F | V101I | — | 1274 | 1275 | 1276 | 1277 |
| UGX182 | parental | S59D, | — | 1278 | 1279 | 1280 | 1281 |
| | | V101I | | | | | |
| UGX183 | S31F, | parental | — | 1282 | 1283 | 1284 | 1285 |
| | S53E | | | | | | |
| UGX184 | N92E, | parental | — | 1286 | 1287 | 1288 | 1289 |
| | D93F | | | | | | |
| UGX185 | S31E, | parental | — | 1290 | 1291 | 1292 | 1293 |
| | S53E | | | | | | |
| UGX186 | S31F, | parental | — | 1294 | 1295 | 1296 | 1297 |
| | D50F | | | | | | |
| UGX187 | parental | N31E, | — | 1298 | 1299 | 1300 | 1301 |
| | | S59E | | | | | |
| UGX188 | parental | S57E, | — | 1302 | 1303 | 1304 | 1305 |
| | | S59D | | | | | |
| UGX189 | parental | N31E, | — | 1306 | 1307 | 1308 | 1309 |
| | | I54F | | | | | |
| UGX190 | parental | S59T, | — | 1310 | 1311 | 1312 | 1313 |
| | | V101L | | | | | |
| UGX191 | parental | I54Y, | — | 1314 | 1315 | 1316 | 1317 |
| | | V101F | | | | | |
| UGX192 | parental | N52F, | — | 1318 | 1319 | 1320 | 1321 |
| | | V101I | | | | | |
| UGX193 | N92D, | parental | — | 1322 | 1323 | 1324 | 1325 |
| | D93F | | | | | | |
| UGX194 | parental | N52F, | — | 1326 | 1327 | 1328 | 1329 |
| | | V101L | | | | | |
| UGX195 | S31F, | parental | — | 1330 | 1331 | 1332 | 1333 |
| | D50E | | | | | | |
| UGX196 | Y94D | S59D | UGX301 | 1334 | 1335 | 1336 | 1337 |
| UGX201 | N92F | S59E | — | 1338 | 1339 | 1340 | 1341 |
| UGX202 | N92Y | S59E | — | 1342 | 1343 | 1344 | 1345 |
| UGX203 | S52D | S59E | — | 1346 | 1347 | 1348 | 1349 |
| UGX204 | S52E | S59E | — | 1350 | 1351 | 1352 | 1353 |
| UGX205 | Y94D | S59E | — | 1354 | 1355 | 1356 | 1357 |
| UGX206 | Y94E | S59E | — | 1358 | 1359 | 1360 | 1361 |
| UGX302 | Y94D | S59Q | — | 1362 | 1363 | 1364 | 1365 |
| UGX303 | Y94E | S59D | — | 1366 | 1367 | 1368 | 1369 |
| UGX304 | Y94E | S59Q | — | 1370 | 1371 | 1372 | 1373 |
| UGX305 | Y94Q | S59D | — | 1374 | 1375 | 1376 | 1377 |
| UGX306 | Y94Q | S59E | — | 1378 | 1379 | 1380 | 1381 |
| UGX307 | Y94Q | S59Q | — | 1382 | 1383 | 1384 | 1385 |
| UGX308 | parental | S59E, | — | 1386 | 1387 | 1388 | 1389 |
| | | I54Y | | | | | |
| UGX309 | parental | S59D, | — | 1390 | 1391 | 1392 | 1393 |
| | | I54Y | | | | | |
| UGX310 | parental | S59Q, | — | 1394 | 1395 | 1396 | 1397 |
| | | I54Y | | | | | |
| UGX311 | Y94D | I54Y | — | 1398 | 1399 | 1400 | 1401 |
| UGX312 | Y94E | I54Y | — | 1402 | 1403 | 1404 | 1405 |
| UGX313 | Y94Q | I54Y | — | 1406 | 1407 | 1408 | 1409 |
| UGX314 | N92Y | S59D | — | 1410 | 1411 | 1412 | 1413 |
| UGX315 | N92Y | S59Q | — | 1414 | 1415 | 1416 | 1417 |
| UGX316 | N92F | S59D | — | 1418 | 1419 | 1420 | 1421 |
| UGX317 | N92F | S59Q | — | 1422 | 1423 | 1424 | 1425 |
| UGX318 | N92L | S59D | — | 1426 | 1427 | 1428 | 1429 |
| UGX319 | N92L | S59E | — | 1430 | 1431 | 1432 | 1433 |
| UGX320 | N92L | S59Q | — | 1434 | 1435 | 1436 | 1437 |
| UGX321 | N92I | S59D | — | 1438 | 1439 | 1440 | 1441 |
| UGX322 | N92I | S59E | — | 1442 | 1443 | 1444 | 1445 |
| UGX323 | N92I | S59Q | — | 1446 | 1447 | 1448 | 1449 |
| UGX324 | N92T | S59D | — | 1450 | 1451 | 1452 | 1453 |
| UGX325 | N92T | S59E | — | 1454 | 1455 | 1456 | 1457 |
| UGX326 | N92T | S59Q | — | 1458 | 1459 | 1460 | 1461 |
| UGX327 | parental | I54A | — | 1462 | 1463 | 1464 | 1465 |
| UGX328 | parental | I54L | — | 1466 | 1467 | 1468 | 1469 |
| UGX329 | parental | I54V | — | 1470 | 1471 | 1472 | 1473 |
| UGX330 | parental | I54R | — | 1474 | 1475 | 1476 | 1477 |
| UGX331 | parental | I54D | — | 1478 | 1479 | 1480 | 1481 |
| UGX332 | parental | I54G | — | 1482 | 1483 | 1484 | 1485 |
| UGX333 | parental | I54H | — | 1486 | 1487 | 1488 | 1489 |
| UGX334 | parental | I54K | — | 1490 | 1491 | 1492 | 1493 |
| UGX335 | parental | I54M | — | 1494 | 1495 | 1496 | 1497 |
| UGX336 | parental | I54N | — | 1498 | 1499 | 1500 | 1501 |
| UGX337 | parental | I54P | — | 1502 | 1503 | 1504 | 1505 |
| UGX338 | parental | I54W | — | 1506 | 1507 | 1508 | 1509 |
| UGX339 | parental | S59A | — | 1510 | 1511 | 1512 | 1513 |
| UGX341 | parental | S59N | — | 1514 | 1515 | 1516 | 1517 |
| UGX342 | parental | S59G | — | 1518 | 1519 | 1520 | 1521 |
| UGX343 | parental | S59H | — | 1522 | 1523 | 1524 | 1525 |
| UGX344 | parental | S59K | — | 1526 | 1527 | 1528 | 1529 |
| UGX345 | parental | S59M | — | 1530 | 1531 | 1532 | 1533 |
| UGX346 | parental | S59P | — | 1534 | 1535 | 1536 | 1537 |
| UGX347 | parental | S59W | — | 1538 | 1539 | 1540 | 1541 |
| UGX348 | Y94A | parental | — | 1542 | 1543 | 1544 | 1545 |
| UGX349 | Y94N | parental | — | 1546 | 1547 | 1548 | 1549 |
| UGX350 | Y94G | parental | — | 1550 | 1551 | 1552 | 1553 |
| UGX351 | Y94H | parental | — | 1554 | 1555 | 1556 | 1557 |
| UGX352 | Y94I | parental | — | 1558 | 1559 | 1560 | 1561 |
| UGX353 | Y94L | parental | — | 1562 | 1563 | 1564 | 1565 |
| UGX354 | Y94K | parental | — | 1566 | 1567 | 1568 | 1569 |
| UGX355 | Y94M | parental | — | 1570 | 1571 | 1572 | 1573 |
| UGX356 | Y94P | parental | — | 1574 | 1575 | 1576 | 1577 |
| UGX357 | Y94W | parental | — | 1578 | 1579 | 1580 | 1581 |
| UGX358 | Y94V | parental | — | 1582 | 1583 | 1584 | 1585 |
| UGX359 | N92A | parental | — | 1586 | 1587 | 1588 | 1589 |
| UGX361 | N92G | parental | — | 1590 | 1591 | 1592 | 1593 |
| UGX362 | N92H | parental | — | 1594 | 1595 | 1596 | 1597 |
| UGX363 | N92I | parental | — | 1598 | 1599 | 1600 | 1601 |
| UGX364 | N92L | parental | — | 1602 | 1603 | 1604 | 1605 |
| UGX365 | N92K | parental | — | 1606 | 1607 | 1608 | 1609 |
| UGX366 | N92M | parental | — | 1610 | 1611 | 1612 | 1613 |
| UGX367 | N92P | parental | — | 1614 | 1615 | 1616 | 1617 |
| UGX368 | N92W | parental | — | 1618 | 1619 | 1620 | 1621 |
| UGX369 | N92V | parental | — | 1622 | 1623 | 1624 | 1625 |
| UGX370 | parental | A103I | — | 1626 | 1627 | 1628 | 1629 |
| UGX371 | parental | A103L | — | 1630 | 1631 | 1632 | 1633 |
| UGX372 | parental | A103R | — | 1634 | 1635 | 1636 | 1637 |
| UGX373 | parental | A103N | — | 1638 | 1639 | 1640 | 1641 |
| UGX374 | parental | A103G | — | 1642 | 1643 | 1644 | 1645 |
| UGX375 | parental | A103H | — | 1646 | 1647 | 1648 | 1649 |
| UGX376 | parental | A103K | — | 1650 | 1651 | 1652 | 1653 |
| UGX377 | parental | A103M | — | 1654 | 1655 | 1656 | 1657 |
| UGX378 | parental | A103P | — | 1658 | 1659 | 1660 | 1661 |
| UGX379 | parental | A103W | — | 1662 | 1663 | 1664 | 1665 |
| UGX380 | parental | A103V | — | 1666 | 1667 | 1668 | 1669 |
| UGX381 | S52D | S59Q | — | 1670 | 1671 | 1672 | 1673 |
| UGX382 | S52D | S59D | — | 1674 | 1675 | 1676 | 1677 |
| UGX383 | S52E | S59Q | — | 1678 | 1679 | 1680 | 1681 |
| UGX384 | S52E | S59D | — | 1682 | 1683 | 1684 | 1685 |
| UGX385 | S52Q | S59E | — | 1686 | 1687 | 1688 | 1689 |
| UGX386 | S52A | parental | — | 1690 | 1691 | 1692 | 1693 |
| UGX388 | S52N | parental | — | 1694 | 1695 | 1696 | 1697 |
| UGX389 | S52G | parental | — | 1698 | 1699 | 1700 | 1701 |
| UGX390 | S52H | parental | — | 1702 | 1703 | 1704 | 1705 |
| UGX391 | S52I | parental | — | 1706 | 1707 | 1708 | 1709 |
| UGX392 | S52L | parental | — | 1710 | 1711 | 1712 | 1713 |
| UGX393 | S52K | parental | — | 1714 | 1715 | 1716 | 1717 |
| UGX394 | S52M | parental | — | 1718 | 1719 | 1720 | 1721 |
| UGX395 | S52P | parental | — | 1722 | 1723 | 1724 | 1725 |
| UGX396 | S52W | parental | — | 1726 | 1727 | 1728 | 1729 |
| UGX397 | S52V | parental | — | 1730 | 1731 | 1732 | 1733 |
| UGX398 | parental | S59E, | — | 1734 | 1735 | 1736 | 1737 |
| | | V101I | | | | | |
| UGX399 | parental | S59D, | — | 1738 | 1739 | 1740 | 1741 |
| | | V101L | | | | | |
| UGX400 | parental | S59Q, | — | 1742 | 1743 | 1744 | 1745 |
| | | V101I | | | | | |
| UGX401 | parental | S59D, | — | 1746 | 1747 | 1748 | 1749 |
| | | V101S | | | | | |
| UGX402 | parental | S59E, | — | 1750 | 1751 | 1752 | 1753 |
| | | V101S | | | | | |
| UGX403 | parental | V101A | — | 1754 | 1755 | 1756 | 1757 |
| UGX405 | parental | V101N | — | 1758 | 1759 | 1760 | 1761 |
| UGX406 | parental | V101D | — | 1762 | 1763 | 1764 | 1765 |
| UGX409 | parental | V101G | — | 1766 | 1767 | 1768 | 1769 |
| UGX410 | parental | V101H | — | 1770 | 1771 | 1772 | 1773 |
| UGX411 | parental | V101K | — | 1774 | 1775 | 1776 | 1777 |
| UGX412 | parental | V101M | — | 1778 | 1779 | 1780 | 1781 |
| UGX413 | parental | V101P | — | 1782 | 1783 | 1784 | 1785 |
| UGX414 | parental | V101S | — | 1786 | 1787 | 1788 | 1789 |
| UGX415 | parental | V101W | — | 1790 | 1791 | 1792 | 1793 |
| UGX416 | parental | I54Y, | — | 1794 | 1795 | 1796 | 1797 |
| | | A103S | | | | | |
| UGX417 | parental | I54F, | — | 1798 | 1799 | 1800 | 1801 |
| | | A103S | | | | | |
| UGX418 | Y94D | A103S | — | 1802 | 1803 | 1804 | 1805 |
| UGX419 | Y94E | A103S | — | 1806 | 1807 | 1808 | 1809 |
| UGX420 | N92F | A103S | — | 1810 | 1811 | 1812 | 1813 |
| UGX421 | N92Y | A103S | — | 1814 | 1815 | 1816 | 1817 |
| UGX422 | S52D | A103S | — | 1818 | 1819 | 1820 | 1821 |
| UGX423 | S52E | A103S | — | 1822 | 1823 | 1824 | 1825 |
| UGX424 | parental | S59D, | — | 1826 | 1827 | 1828 | 1829 |
| | | A103S | | | | | |
| UGX425 | parental | S59E, | — | 1830 | 1831 | 1832 | 1833 |
| | | A103S | | | | | |
| UGX426 | parental | S59Q, | — | 1834 | 1835 | 1836 | 1837 |
| | | A103S | | | | | |
| UGX427 | N92Y | I54Y | — | 1838 | 1839 | 1840 | 1841 |
| UGX428 | N92F | I54Y | — | 1842 | 1843 | 1844 | 1845 |
| UGX429 | parental | V101I, | — | 1846 | 1847 | 1848 | 1849 |
| | | I54F | | | | | |
| UGX430 | parental | V101I, | — | 1850 | 1851 | 1852 | 1853 |
| | | I54Y | | | | | |

What is claimed is:

1. An isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 but comprising one or more amino acid change relative to SEQ ID NO: 3, wherein the one or more amino acid change is selected from T30E, T30F, T30Q, T30S, T30Y, N31D, N31E, N31F, N31Q, N31R, N31Y, H32E, H32F, H32Q, H32S, H32T, I50D, 150E, 150F, 150S, 150T, I50Y, N52E, N52F, N52Q, N52Y, I54E, I54F, I54L, I54Q, I54S, I54T, I54V, I54Y, S55E, S55F, S55Q, S55T, S55Y, S59D, S59E, S59F, S59I, S59L, S59Q, S59T, S59V, S59Y, D99E, D99Q, D99Y, I100D, I100E, I100F, I100L, I100Q, I100V, I100Y, V101F, V101,I V101L, V101T, V101Y, D102E, D102F, D102I, D102L, D102Q, D102Y, A103D, A103E, A103F, A103Q, A103S, A103T, and A103Y.

2. An isolated antibody or a fragment thereof that specifically binds to a human fibroblast growth factor 23 (FGF23) polypeptide, wherein the antibody or fragment thereof comprises a light chain variable region (VL) amino acid sequence according to SEQ ID NO: 4 but comprising one or more amino acid change relative to SEQ ID NO: 4, wherein the one or more amino acid change is selected from A25L, A25V, I29F, I29L, I29V, S30D, S30E, S30F, S30I, S30L, S30Q, S30T, S30V, S30Y, S31D, S31E, S31F, S31I, S31L, S31Q, S31T, S31V, S31Y, A32F, A32I, A32L, A32T, A32V, A32Y, V34F, V34I, V34L, V34Y, D50E, D50F, D50I, D50L, D50Q, D50R, D50S, D50T, D50V, A51F, A51I, A51L, A51S, A51T, A51V, S52D, S52E, S52F, S52Q, S52T, S52Y, S53D, S53E, S53F, 5531, S53L, S53Q, S53R, S53T, S53V, S53Y, Q90F, Q90Y, F91K, F91R, F91Y, N92D, N92E, N92F, N92Q, N92S, N92T, N92Y, D93E, D93F, D93Q, D93R, D93S, D93T, D93Y, Y94D, Y94E, Y94F, Y94Q, Y94S, and Y94T.

3. An isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VH amino acid sequence comprises one or more amino acid change selected from T30E, T30F, T30Q, T30S, T30Y, N31D, N31E, N31F, N31Q, N31R, N31Y, H32E, H32F, H32Q, H32S, H32T, I50D, 150E, I50F, I50S, 150T, 150Y, N52E, N52F, N52Q, N52Y, I54E, I54F, I54L, I54Q, I54S, I54T, I54V, I54Y, S55E, S55F, S55Q, S55T, S55Y, S59D, S59E, S59F, S59I, S59L, S59Q, S59T, S59V, S59Y, D99E, D99Q, D99Y, I100D, I100E, I100F, I100L, I100Q, I100V, I100Y, V101F, V101I, V101L, V101T, V101Y, D102E, D102F, D102I, D102L, D102Q, D102Y, A103D, A103E, A103F, A103Q, A103S, A103T, and A103Y, and wherein the VL amino acid sequence comprises one or more amino acid change selected from A25L, A25V, 129F, 129L, 129V, S30D, S30E, S30F, 5301, S30L, S30Q, S30T, S30V, S30Y, S31D, S31E, S31F, S31I, S31L, S31Q, S31T, S31V, S31Y, A32F, A32I, A32L, A32T, A32V, A32Y, V34F, V34I, V34L, V34Y, D50E, D50F, D50I, D50L, D50Q, D50R, D50S, D50T, D50V, A51F, A51I, A51L, A51S, A51T, A51V, S52D, S52E, S52F, S52Q, S52T, S52Y, S53D, S53E, S53F, S53I, S53L, S53Q, S53R, S53T, S53V, S53Y, Q90F, Q90Y, F91K, F91R, F91Y, N92D, N92E, N92F, N92Q, N92S, N92T, N92Y, D93E, D93F, D93Q, D93R, D93S, D93T, D93Y, Y94D, Y94E, Y94F, Y94Q, Y94S, and Y94T.

4. An isolated antibody or a fragment thereof that specifically binds to a human fibroblast growth factor 23 (FGF23) polypeptide, wherein the antibody or fragment thereof comprises a heavy chain complementarity determining region 1 (HCDR1) according to an amino acid sequence selected from SEQ ID NOs: 22-32.

5. An isolated antibody or a fragment thereof that specifically binds to a human fibroblast growth factor 23 (FGF23) polypeptide, wherein the antibody or fragment thereof comprises a heavy chain complementarity determining region 2 (HCDR2) according to an amino acid sequence selected from SEQ ID NOs: 33-65.

6. An isolated antibody or a fragment thereof that specifically binds to a human fibroblast growth factor 23 (FGF23) polypeptide, wherein the antibody or fragment thereof comprises a heavy chain complementarity determining region 3 (HCDR3) according to an amino acid sequence selected from SEQ ID NOs: 66-93.

7. An isolated antibody or a fragment thereof that specifically binds to a human fibroblast growth factor 23 (FGF23) polypeptide, wherein the antibody or fragment thereof comprises a light chain complementarity determining region 1 (LCDR1) according to an amino acid sequence selected from SEQ ID NOs: 94-126.

8. An isolated antibody or a fragment thereof that specifically binds to a human fibroblast growth factor 23 (FGF23) polypeptide, wherein the antibody or fragment thereof comprises a light chain complementarity determining region 2 (LCDR2) according to an amino acid sequence selected from SEQ ID NOs: 127-158.

9. An isolated antibody or a fragment thereof that specifically binds to a human fibroblast growth factor 23 (FGF23) polypeptide, wherein the antibody or fragment thereof comprises a light chain complementarity determining region 3 (LCDR3) according to an amino acid sequence selected from SEQ ID NOs: 159-185.

10. An isolated antibody or a fragment thereof that specifically binds to a human fibroblast growth factor 23 (FGF23) polypeptide, wherein the antibody or fragment thereof comprises a HCDR1, a HCDR2, a HCDR3, a LCDR1, a LCDR2, and a LCDR3, wherein:
the HCDR1 comprises an amino acid sequence selected from SEQ ID NO: 11 and SEQ ID NOs: 22-32;
the HCDR2 comprises an amino acid sequence selected from SEQ ID NO: 12 and SEQ ID NOs: 33-65;
the HCDR3 comprises an amino acid sequence selected from SEQ ID NO: 13 and SEQ ID NOs: 66-93;
the LCDR1 comprises an amino acid sequence selected from SEQ ID NO: 14 and SEQ ID NOs: 94-126;
the LCDR2 comprises an amino acid sequence selected from SEQ ID NO: 15 and SEQ ID NOs: 127-158; and
the LCDR3 comprises an amino acid sequence selected from SEQ ID NO: 16 and SEQ ID NOs: 159-185.

11. The isolated antibody or fragment thereof of any one of claims 1-10, wherein the antibody or fragment thereof comprises a heavy chain variable region (VH) and a light chain variable region (VL),
wherein the VH comprises an amino acid sequence having at least 80% sequence identity to an amino acid sequence selected from SEQ ID NO: 3 and SEQ ID NOs: 186-270, or
wherein the VL comprises an amino acid sequence having at least 80% sequence identity to an amino acid sequence selected from SEQ ID NO: 4 and SEQ ID NOs: 271-368.

12. The isolated antibody or fragment thereof of any one of claims 1-11, wherein
the antibody or fragment thereof comprises a VH comprising an amino acid sequence having at least 90% sequence identity to an amino acid sequence selected from SEQ ID NO: 3 and SEQ ID NOs: 186-270, or
the antibody or fragment thereof comprises a VL comprising an amino acid sequence having at least 90% sequence identity to an amino acid sequence selected from SEQ ID NO: 4 and SEQ ID NOs: 271-368.

13. The isolated antibody or fragment thereof of any one of claims 1-12, wherein
the antibody or fragment thereof comprises a VH comprising an amino acid sequence having at least 95% sequence identity to an amino acid sequence selected from SEQ ID NO: 3 and SEQ ID NOs: 186-270, or
the antibody or fragment thereof comprises a VL comprising an amino acid sequence having at least 95% sequence identity to an amino acid sequence selected from SEQ ID NO: 4 and SEQ ID NOs: 271-368.

14. The isolated antibody or fragment thereof of any one of claims 1-13, wherein the antibody or fragment thereof comprises a VH and a VL, and wherein
the VH comprises an amino acid sequence having at least 80% sequence identity to an amino acid sequence selected from SEQ ID NO: 3 and SEQ ID NOs: 186-270, and
the VL comprises an amino acid sequence having at least 80% sequence identity to an amino acid sequence selected from SEQ ID NO: 4 and SEQ ID NOs: 271-368.

15. The isolated antibody or fragment thereof of any one of claims 1-14, wherein the antibody or fragment thereof comprises a VH and a VL, and wherein
the VH comprises an amino acid sequence having at least 90% sequence identity to an amino acid sequence selected from SEQ ID NO: 3 and SEQ ID NOs: 186-270, and
the VL comprises an amino acid sequence having at least 90% sequence identity to an amino acid sequence selected from SEQ ID NO: 4 and SEQ ID NOs: 271-368.

16. The isolated antibody or fragment thereof of any one of claims 1-15, wherein the antibody or fragment thereof comprises a VH and a VL, and wherein
the VH comprises an amino acid sequence having at least 95% sequence identity to an amino acid sequence selected from SEQ ID NO: 3 and SEQ ID NOs: 186-270, and
the VL comprises an amino acid sequence having at least 95% sequence identity to an amino acid sequence selected from SEQ ID NO: 4 and SEQ ID NOs: 271-368.

17. The isolated antibody or fragment thereof of any one of claims 1-16, wherein the antibody or fragment thereof comprises a VH and a VL, and wherein
the VH comprises an amino acid sequence selected from SEQ ID NO: 3 and SEQ ID NOs: 186-270, or
the VL comprises an amino acid sequence selected from SEQ ID NO: 4 and SEQ ID NOs: 271-368.

18. The isolated antibody or fragment thereof of any one of claims 1-16, wherein the antibody or fragment thereof comprises a VH and a VL, and wherein
the VH comprises an amino acid sequence selected from SEQ ID NO: 3 and SEQ ID NOs: 186-270, and
the VL comprises an amino acid sequence selected from SEQ ID NO: 4 and SEQ ID NOs: 271-368.

19. The isolated antibody or fragment thereof of any one of claims 1-18, wherein the antibody or fragment thereof comprises a heavy chain variable region (VH) amino acid sequence according to SEQ ID NO: 3 and a light chain variable region (VL) according to SEQ ID NO: 4, but comprising at least two amino acid changes in the VH and/or VL sequences, wherein the at least two amino acid changes are selected from VL:S31E/VL:D50F, VH:N31F/VH:S59F, VL:D50F/VL:S53E, VL:S31F/VL:S52E, VL:D50F/VH:V101I, VH:S59D/VH:V101I, VL:S31F/VL:S53E, VL:N92E/VL:D93F, VL:S31E/VL:S53E, VL:S31F/VL:D50F, VH:N31E/VH:S59E, VH:S57E/VH:S59D, VH:N31E/VH:I54F, VH:S59T/VH:V101L, VH:I54Y/VH:V101F, VH:N52F/VH:V101I, VL:N92D/VL:D93F, VH:N52F/VH:V101L, VL:S31F/VL:D50E, VL:Y94D/VH:S59D, VL:Y94D/VH:S59E, VL:N92F/VH:S59E, VL:N92Y/VH:S59E, VL:Y94E/VH:S59E, VL:S52D/VH:S59E, and VL:S52E/VH:S59E.

20. An isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VH amino acid sequence comprises one or more amino acid change selected from I54F, I54Y, S59D, S59E, V101I, and A103S compared to SEQ ID NO: 3.

21. An isolated antibody or a fragment thereof that specifically binds to a human fibroblast growth factor 23 (FGF23) polypeptide, wherein the antibody or fragment thereof comprises a VH amino acid sequence according to SEQ ID NO: 3 and a VL amino acid sequence according to SEQ ID NO: 4, wherein the VL amino acid sequence comprises one or more amino acid change selected from S52D, S52E, N92F, N92Y, Y94D, and Y94E compared to SEQ ID NO: 4.

22. An isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-027 (UGX126).

23. An isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-066 (UGX144).

24. An isolated antibody or a fragment thereof that specifically binds to a human fibroblast growth factor 23 (FGF23) polypeptide, wherein the antibody or fragment thereof comprises a heavy chain variable region (VH) according to VH-001 and a light chain variable region (VL) according to VL-067 (UGX145).

25. An isolated antibody or a fragment thereof that specifically binds to a human fibroblast growth factor 23 (FGF23) polypeptide, wherein the antibody or fragment thereof comprises a heavy chain variable region (VH) according to VH-028 and a light chain variable region (VL) according to VL-001 (UGX156).

26. An isolated antibody or a fragment thereof that specifically binds to a human fibroblast growth factor 23 (FGF23) polypeptide, wherein the antibody or fragment thereof comprises a heavy chain variable region (VH) according to VH-029 and a light chain variable region (VL) according to VL-001 (UGX157).

27. An isolated antibody or a fragment thereof that specifically binds to a human fibroblast growth factor 23 (FGF23) polypeptide, wherein the antibody or fragment thereof comprises a heavy chain variable region (VH) according to VH-061 and a light chain variable region (VL) according to VL-001 (UGX175).

28. An isolated antibody or a fragment thereof that specifically binds to a human fibroblast growth factor 23 (FGF23) polypeptide, wherein the antibody or fragment thereof comprises a heavy chain variable region (VH) according to VH-055 and a light chain variable region (VL) according to VL-001 (UGX182).

29. An isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-050 (UGX201).

30. An isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-054 (UGX202).

31. An isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-027 (UGX203).

32. An isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-030 (UGX204).

33. An isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-066 (UGX205).

34. An isolated antibody or a fragment thereof that specifically binds to a human fibroblast growth factor 23 (FGF23) polypeptide, wherein the antibody or fragment thereof comprises a heavy chain variable region (VH) according to VH-031 and a light chain variable region (VL) according to VL-067 (UGX206).

35. An isolated antibody or a fragment thereof that specifically binds to a human fibroblast growth factor 23 (FGF23) polypeptide, wherein the antibody or fragment thereof comprises a heavy chain (HC) amino acid sequence according to SEQ ID NO: 1 and a light chain (LC) amino acid sequence according to SEQ ID NO: 2, wherein the antibody or fragment thereof comprises one or more HC amino acid change relative to SEQ ID NO: 1 and/or one or more LC amino acid change relative to SEQ ID NO: 2, wherein the one or more HC amino acid change is selected from T28Y, T30F, T30K, N31D, N31E, N31F, N31Q, N31R, H32K, H32Y, Y33D, Y33L, Y33R, N52E, N52F, N52Q, N52R, N52Y, I54A, I54D, I54F, I54G, I54H, I54K, I54L, I54M, I54N, I54P, I54R, I54V, I54W, I54Y, S55E, S55F, S55I, S55Q, S55R, S55Y, S57E, S57F, S57R, S57Y, T58F, T58R, S59A, S59D, S59E, S59F, S59G, S59H, S59I, S59K, S59L, S59M, S59N, S59P, S59Q, S59R, S59T, S59V, S59W, S59Y, N60E, N60R, N60Y, A61F, A61R, A61Y, Q62L, Q62R, Q62Y, Q65E, Q65R, Q65Y, D99F, D99R, D99Y, I100D, I100E, I100F, I100Y, V101A, V101D, V101E, V101F, V101G, V101H, V101I, V101K, V101L, V101M, V101N, V101P, V101Q, V101R, V101S, V101W, D102E, D102F, D102K, D102R, D102Y, A103D, A103F, A103G, A103H, A103I, A103K, A103L, A103M, A103N, A103P, A103R, A103S, A103T, A103V, and A103W, and the one or more LC amino acid change is selected from Q27F, Q27R, G28D, G28Q, G28R, G28Y, 129F, I29L, S30D, S30E, S30F, S30L, S30Q, S30R, S30Y, S31D, S31E, S31F, S31Q, S31Y, A32F, A32V, V34F, V34I, D50E, D50F, D50L, D50Q, D50R, D50S, D50T, D50V, D50Y, A51V, S52A, S52D, S52E, S52F, S52G, S52H, S52I, S52K, S52L, S52M, S52N, S52P, S52Q, S52R, S52V, S52W, S52Y, S53D, S53E, S53F, S53R, S53Y, L54E, L54Y, E55R, E55Y, S67E, S67R, S67Y, T69R, T69Y, F91E, F91R, F91Y, N92A, N92D, N92E, N92F, N92G, N92H, N92I, N92K, N92L, N92M, N92P, N92Q, N92R, N92S, N92T, N92V, N92W, N92Y, D93F, D93Q, D93R, D93S, D93T, D93Y, Y94A, Y94D, Y94E, Y94F, Y94G, Y94H, Y94I, Y94K, Y94L, Y94M, Y94N, Y94P, Y94Q, Y94R, Y94S, Y94V, and Y94W.

36. An isolated antibody or a fragment thereof that specifically binds to a human fibroblast growth factor 23 (FGF23) polypeptide, wherein the antibody or fragment thereof comprises a heavy chain variable region (VH) amino acid sequence according to SEQ ID NO: 3 and a light chain variable region (VL) amino acid sequence according to SEQ ID NO: 4, wherein the antibody or fragment thereof comprises one or more VH amino acid change relative to SEQ ID NO: 3 and/or one or more VL amino acid change relative to SEQ ID NO: 4, wherein the one or more VH amino acid change is selected from T28Y, T30F, T30K, N31D, N31E, N31F, N31Q, N31R, H32K, H32Y, Y33D, Y33L, Y33R, N52E, N52F, N52Q, N52R, N52Y, I54A, I54D, I54F, I54G, I54H, I54K, I54L, I54M, I54N, I54P, I54R, I54V, I54W, I54Y, S55E, S55F, S55I, S55Q, S55R, S55Y, S57E, S57F, S57R, S57Y, T58F, T58R, S59A, S59D, S59E, S59F, S59G, S59H, S59I, S59K, S59L, S59M, S59N, S59P, S59Q, S59R, S59T, S59V, S59W, S59Y, N60E, N60R, N60Y, A61F, A61R, A61Y, Q62L, Q62R, Q62Y, Q65E, Q65R, Q65Y, D99F, D99R, D99Y, I100D, I100E, I100F, I100Y, V101A, V101D, V101E, V101F, V101G, V101H, V101I, V101K, V101L, V101M, V101N, V101P, V101Q, V101R, V101S, V101W, D102E, D102F, D102K, D102R, D102Y, A103D, A103F, A103G, A103H, A103I, A103K, A103L, A103M, A103N, A103P, A103R, A103S, A103T, A103V, and A103W, and the one or more VL amino acid change is selected from Q27F, Q27R, G28D, G28Q, G28R, G28Y, 129F, 129L, S30D, S30E, S30F, S30L, S30Q, S30R, S30Y, S31D, S31E, S31F, S31Q, S31Y, A32F, A32V, V34F, V34I, D50E, D50F, D50L, D50Q, D50R, D50S, D50T, D50V, D50Y, A51V, S52A, S52D, S52E, S52F, S52G, S52H, S52I, S52K, S52L, S52M, S52N, S52P, S52Q, S52R, S52V, S52W, S52Y, S53D, S53E, S53F, S53R, S53Y, L54E, L54Y, E55R, E55Y, S67E, S67R, S67Y, T69R, T69Y, F91E, F91R, F91Y, N92A, N92D, N92E, N92F, N92G, N92H, N92I, N92K, N92L, N92M, N92P, N92Q, N92R, N92S, N92T, N92V, N92W, N92Y, D93F, D93Q, D93R, D93S, D93T, D93Y, Y94A, Y94D, Y94E, Y94F, Y94G, Y94H, Y94I, Y94K, Y94L, Y94M, Y94N, Y94P, Y94Q, Y94R, Y94S, Y94V, and Y94W.

37. An isolated antibody or a fragment thereof that specifically binds to a human fibroblast growth factor 23 (FGF23) polypeptide with greater binding affinity compared to burosumab, wherein the antibody or fragment thereof comprises a heavy chain variable region (VH) amino acid sequence and a light chain variable region (VL) amino acid sequence according to Table 22.

38. An isolated antibody or a fragment thereof that specifically binds to a human fibroblast growth factor 23 (FGF23) polypeptide with greater binding affinity compared to burosumab, wherein the antibody or fragment thereof comprises a heavy chain (HC) amino acid sequence and a light chain (LC) amino acid sequence according to Table 22.

39. The isolated antibody or fragment thereof of any one of claims 1-38, wherein the antibody or fragment thereof has increased affinity for human FGF23 as compared to burosumab.

40. The isolated antibody or fragment thereof of any one of claims 1-39, wherein the antibody or fragment thereof has about 2-fold to about 10-fold increased binding affinity for human FGF23 compared to burosumab.

41. The isolated antibody or fragment thereof of any one of claims 1-40, wherein the binding affinity of the antibody or fragment thereof to human FGF23 (K_{D}) is between about 1×10⁻¹² M to about 7×10⁻¹² M.

42. The isolated antibody or fragment thereof of any one of claims 1-41, for use in a method of decreasing serum FGF23 in a subject in need thereof.

43. The isolated antibody or fragment thereof of any one of claims 1-41, for use in a method of increasing renal maximum threshold for phosphate reabsorption (TmP) in a subject in need thereof.

44. The isolated antibody or fragment thereof of any one of claims 1-41, for use in a method of increasing serum inorganic phosphorus (Pi) in a subject in need thereof.

45. The isolated antibody or fragment thereof of any one of claims 1-41, for use in a method of increasing serum 1,25 dihydroxyvitamin D (1,25[OH]2D) concentration in a subject in need thereof.

46. The isolated antibody or fragment thereof of any one of claims 1-41, for use in a method of treating a hypophosphatemic condition in subject in need thereof.

47. The isolated antibody or fragment thereof for use of any one of claims 42-46, wherein the subject is diagnosed with X-linked hypophosphatemia (XLH) or tumor-induced osteomalacia (TIO).

48. A method of decreasing serum FGF23 in a subject in need thereof, the method comprising administering the isolated antibody or fragment thereof of any one of claims 1-41 to the subject.

49. A method of increasing renal maximum threshold for phosphate reabsorption (TmP) in a subject in need thereof, the method comprising administering the isolated antibody or fragment thereof of any one of claims 1-41 to the subject.

50. A method of increasing serum inorganic phosphorus (Pi) in a subject in need thereof, the method comprising administering the isolated antibody or fragment thereof of any one of claims 1-41 to the subject.

51. A method of increasing serum 1,25 dihydroxyvitamin D (1,25[OH]2D) concentration in a subject in need thereof, the method comprising administering the isolated antibody or fragment thereof of any one of claims 1-41 to the subject.

52. A method of treating a hypophosphatemic condition in a subject in need thereof, the method comprising administering the isolated antibody or fragment thereof of any one of claims 1-41 to the subject.

53. The method of any one of claims 48-52, wherein the subject is diagnosed with X-linked hypophosphatemia (XLH) or tumor-induced osteomalacia (TIO).
